(19) United States
(12) Patent Application Publication
(10) Pub. No.: US 2025/0144057 A1
(43) Pub. Date: May 8, 2025

(54) GAMMA-HYDROXYBUTYRATE COMPOSITIONS HAVING IMPROVED PHARMACOKINETICS IN THE FED STATE

(71) Applicant: Flamel Ireland Limited, Dublin (IE)

(72) Inventors: Julien Grassot, Lyon (FR); Cendrine Grangeon, Villeurbanne (FR); Jordan Dubow, Chicago, IL (US); Jason Vaughn, St. Louis, MO (US)

(21) Appl. No.: 19/013,881

(22) Filed: Jan. 8, 2025

Related U.S. Application Data
(63) Continuation-in-part of application No. 18/898,132, filed on Sep. 26, 2024.
Application No. 18/898,132 is a continuation of application No. 18/758,344, filed on Jun. 28, 2024, now Pat. No. 12,167,991.
Application No. 18/758,344 is a continuation of application No. 17/837,740, filed on Jun. 10, 2022, now Pat. No. 12,226,377.
Application No. 17/837,740 is a continuation of application No. 16/804,966, filed on Feb. 28, 2020, now Pat. No. 11,400,065.
(60) Provisional application No. 62/812,699, filed on Mar. 1, 2019.
(60) Provisional application No. 62/857,008, filed on Jun. 4, 2019.

(51) Int. Cl. A61K 31/19; A61K 9/00

(52) U.S. Cl. CPC A61K 31/19; A61K 9/0053; A61K 9/009; A61K 9/0095

(57) ABSTRACT

Oral pharmaceutical compositions of sodium oxybate having improved pharmacokinetic properties when administered less than two hours after eating are provided, and therapeutic uses thereof.

### CROSS-REFERENCE TO RELATED APPLICATIONS

This application is a continuation-in-part of U.S. Non-Provisional application Ser. No. 18/898,132, filed Sep. 26, 2024 which is a continuation of U.S. Non-Provisional application Ser. No. 18/758,344, filed Jun. 28, 2024 which is a continuation of U.S. Non-Provisional application Ser. No. 17/837,740, filed Jun. 10, 2022 which is a continuation of U.S. Non-Provisional application Ser. No. 16/804,966, filed Feb. 28, 2020 which claims priority to U.S. Provisional Application No. 62/812,699, filed Mar. 1, 2019 and U.S. Provisional Application No. 62/857,008, filed Jun. 4, 2019.

### FIELD

The present invention relates to compositions for the treatment of narcolepsy, cataplexy, or excessive daytime sleepiness comprising gamma-hydroxybutyrate in a unit dose suitable for administration less than two hours after eating. The present invention also relates to modified release formulations of gamma-hydroxybutyrate having improved pharmacokinetic (PK) properties in the fed state, and to therapeutic uses thereof.

### BACKGROUND

Narcolepsy is a devastating disabling condition. The cardinal symptoms are excessive daytime sleepiness (EDS), cataplexy (a sudden loss of muscle tone triggered by strong emotions, seen in approximately 60% of patients), hypnogogic hallucination (HH), sleep paralysis (SP), and disturbed nocturnal sleep (DNS). Other than EDS, DNS is the most common symptom seen among narcolepsy patients.

One of the major treatments for narcolepsy is sodium oxybate. The precise mechanism by which sodium oxybate produces an effect is unknown, however sodium oxybate is thought to act by promoting SWS (delta sleep) and consolidating night-time sleep. Sodium oxybate administered before nocturnal sleep increases Stages 3 and 4 sleep and increases sleep latency, while reducing the frequency of sleep onset REM periods (SOREMPs). Other mechanisms, which have yet to be elucidated, may also be involved.

Sodium oxybate is also known as sodium 4-hydroxybutanoate, or gamma-hydroxybutyric acid sodium salt, and has the following chemical structure:

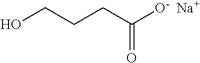

Sodium oxybate is marketed commercially in the United States as Xyrem®. The product is formulated as an immediate release liquid solution that is taken once immediately before bed, and a second time approximately 2.5 to 4 hours later, in equal doses. Sleep-onset may be dramatic and fast, and patients are advised to be sitting in bed when consuming the dose. The most commonly reported side effects are nausea, dizziness, vomiting, somnolence, enuresis and tremor.

One critical drawback of Xyrem® is the requirement to take the first does at least 2 hours after eating. Specifically, Xyrem®'s label expressly advises “[t]ake the first dose of Xyrem® at least 2 hours after eating because food significantly reduces the bioavailability of sodium oxybate.” The medical problem cautioned against by the Xyrem® label and unaddressed by the prior art is a deleterious food effect on the absorption of GHB. As noted in the FDA's Xyrem® Risk Management Program, “Because food significantly reduces the bioavailability of sodium oxybate, the patient should try to eat well before (several hours) going to sleep and taking the first dose of sodium oxybate. Patients should try to minimize variability in the timing of dosing in relation to meals.” (See https://www.accessdata.fda.gov/drugsatfda_docs/label/2002/21196lbl.pdf). As a practical matter, the food effect obstacle of the prior art creates a variety of unmet problems for the patient because it is not always possible to eat several hours before sleep. Likewise, despite best efforts, a patient may have variability in the timing of dosing in relation to meals due to unforeseen schedule changes, travel, etc. As a result, the food effect problem of the prior art necessarily causes reduced patient compliance, reduced efficacy, and reduced safety. Moreover, since GHB is a known drug of abuse, there is inherently a greater risk of abuse, including accidental abuse, as a result of Xyrem®'s food effect problem.

Scientific studies have shown that after a high-fat meal the intestinal uptake of GHB may be significantly decreased due to the inhibition of anionic transporters, such as the Monocarboxylic Acid Transporters (MCTs). MCTs are transport proteins that determine the absorption, renal clearance, and distribution of GHB throughout the body. Recent studies have shown the MCT-mediated intestinal absorption of GHB to occur in a concentration- and proton gradient-dependent manner and via a carrier-mediated process along the length of the intestine. The MCT-mediated intestinal absorption of GHB is important for its pharmacological activity because more than 99% of GHB is ionized and cannot diffuse across cellular membranes at physiologic pH.

Accordingly, there is a need for compositions of gamma-hydroxybutyrate that can be administered less than two hours after eating without compromising safety or efficacy.

### SUMMARY OF THE INVENTION

In an aspect, the present disclosure encompasses an oral pharmaceutical composition for the treatment of narcolepsy, cataplexy, or excessive daytime sleepiness that may be administered less than two hours after eating. For example, a composition including gamma-hydroxybutyrate may be in a unit dose suitable for administration less than two hours after eating. In additional aspects, the composition may me suitable for once-daily administration. In some aspects, the composition provides a mean AUCᵢₙₜ and/or mean Cₘₐₓ when administered less than two hour after eating that is 50%-120% of the mean AUCᵢₙₜ or mean Cₘₐₓ when the composition is administered at least two hours after eating. When administered less than two hours after eating, the composition may provide an AUCᵢₙₜ and/or a Cₘₐₓ bioequivalent to an AUCᵢₙₜ or Cₘₐₓ of an equal dose of immediate release liquid solution of sodium oxybate administered at t₀ and t₄ₕ in equally divided doses at least two hours after eating. In some aspects, a 6 g dose of the composition administered less than two hours after eating has been shown to achieve a mean AUCᵢₙₜ of greater than 240 hr*μ/mL, and a mean Cₘₐₓ that is from 50% to 140% of the mean Cₘₐₓ provided by an equal dose of immediate release liquid solution of sodium oxybate administered at t₀ and t₄ₕ in equally divided doses at least two hours after eating.

Further provided herein is a method of treating narcolepsy and associated disorders and symptoms in a patient in need thereof by administering an oral pharmaceutical composition of gamma-hydroxybutyrate less than two hours after eating. In an aspect, the composition may be administered once-daily. In some aspects, the composition may be administered in the morning or the evening after eating. In other aspects, sleep is includes for at least six consecutive hours.

In additional aspects, a method of treating narcolepsy and associated disorders and symptoms in a patient in need thereof may include administering an oral pharmaceutical composition comprising gamma-hydroxybutyrate once daily, where the composition is dose proportional. The Cₘₐₓ of the composition may be dose proportional across one or more of 4.5 g, 6.0 g, 7.5 g, and 9 g doses of the composition.

Also provided herein are oral pharmaceutical compositions and methods thereof for the treatment of narcolepsy, cataplexy, or excessive daytime sleepiness comprising gamma-hydroxybutyrate in a unit dose suitable for administration once daily, where most adverse events (AEs) occur during the Tₘₐₓ period (around Cₘₐₓ). In another aspect, compositions and methods are provided for the treatment of narcolepsy, cataplexy, or excessive daytime sleepiness comprising gamma-hydroxybutyrate in a unit dose suitable for administration once daily, wherein most adverse events (AEs) occur close to Tₘₐₓ, during the Cₘₐₓ period. In some embodiments of this aspect, administration of the oral pharmaceutical composition less than two hours after eating may result in fewer AEs than administration of the oral pharmaceutical composition at least two hours after eating. The oral pharmaceutical composition of some embodiments may have a more favorable safety profile (i.e., fewer AEs) as compared to an equal dose of immediate release liquid solution of sodium oxybate administered at t₀ and t₄ₕ in equally divided doses.

Other aspects and iterations of the invention are described more thoroughly below.

### DESCRIPTION OF THE FIGURES

The accompanying drawings, which are incorporated in and constitute a part of this specification, illustrate several embodiments of the invention and together with the description serve to explain the principles of the invention.

FIG. 1A depicts the qualitative and quantitative structure of the immediate release (IR) microparticles of gamma-hydroxybutyrate of Example 1.

FIG. 1B depicts the qualitative and quantitative structure of the modified release (MR) microparticles of gamma-hydroxybutyrate of Example 1.

FIG. 2A is a mean concentration versus time curve for 6 g FT218 administered in the fed and fasted state.

FIG. 2B is a series of individual profiles in a mean concentration versus time curve for 6 g FT218 administered in the fed and fasted state.

FIG. 3A is a simulated concentration versus time curve for 3 g IR microparticles of FT218 administered in the fed and fasted state.

FIG. 3B is a simulated concentration versus time curve for 6 g FT218 and IR and MR microparticle portions of FT218 administered in the fed and fasted state.

FIG. 4A is a concentration versus time curve for a 4.5 g single dose of Xyrem® in the fed state, the fasted state and 2 hours post meal.

FIG. 4B is a concentration versus time curve for a 6 g dose of FT218 in the fed state, the fasted state and 2 hours post meal.

FIG. 5A shows expected concentration versus time curves for two 3 g doses of Xyrem® and one 6 g dose of FT218 in the fed state.

FIG. 5B shows expected concentration versus time curves for two 3 g doses of Xyrem® and one 6 g does of FT218 administered two hours post-meal.

FIG. 5C shows expected concentration versus time curves for two 3 g doses of Xyrem® and one 6 g dose of FT218 administered in the fasted state.

FIG. 6A shows expected concentration versus time curves for two 4.5 g doses of Xyrem® and one 9 g dose of FT218 in the fed state.

FIG. 6B shows expected concentration versus time curves for two 4.5 g doses of Xyrem® and one 9 g dose of FT218 administered two hours post-meal.

FIG. 6C shows expected concentration versus time curves for two 4.5 g doses of Xyrem® and one 9 g dose of FT218 administered in the fasted state.

FIG. 7A shows AUC versus time after a meal for 6 g FT218 and 6 g Xyrem®.

FIG. 7B shows Cₘₐₓ versus time after a meal for 6 g FT218 and 6 g Xyrem®.

FIG. 8 shows FT218 plasma concentration time curves for rising doses from 4.5 g to 9 g per night.

FIG. 9 shows dissolution results for a 4.5 g dose in lemon pudding, vanilla yogurt, and applesauce as compared to control.

FIG. 10 shows dissolution results for a 9.0 g dose in lemon pudding, vanilla yogurt, and applesauce as compared to control.

### DETAILED DESCRIPTION

The present invention may be understood more readily by reference to the following detailed description of embodiments of the formulation, methods of treatment using some embodiments of the formulation, and the Examples included therein.

### Definitions and Use of Terms

Wherever an analysis or test is required to understand a given property or characteristic recited herein, it will be understood that the analysis or test is performed in accordance with applicable guidances, draft guidances, regulations and monographs of the United States Food and Drug Administration (“FDA”) and United States Pharmacopoeia (“USP”) applicable to drug products in the United States in force as of Nov. 1, 2015 unless otherwise specified. Clinical endpoints may be judged with reference to standards adopted by the American Academy of Sleep Medicine, including standards published at C Iber, S Ancoli-Israel, A Chesson, SF Quan. The AASM Manual for the Scoring of Sleep and Associated Events. Westchester, IL: American Academy of Sleep Medicine; 2007.

When a pharmacokinetic comparison is made between a formulation described or claimed herein and a reference product, it will be understood that the comparison is performed in a suitable designed cross-over trial, although it will also be understood that a cross-over trial is not required unless specifically stated. It will also be understood that the comparison may be made either directly or indirectly. For example, even if a formulation has not been tested directly against a reference formulation, it can still satisfy a comparison to the reference formulation if it has been tested against a different formulation, and the comparison with the reference formulation may be deduced therefrom.

As used in this specification and in the claims which follow, the singular forms “a,” “an” and “the” include plural referents unless the context dictates otherwise. Thus, for example, reference to “an ingredient” includes mixtures of ingredients, reference to “an active pharmaceutical agent” includes more than one active pharmaceutical agent, and the like.

“Bioavailability” means the rate and extent to which the active ingredient or active moiety is absorbed from a drug product and becomes available at the site of action.

“Relative bioavailability” or “Rel BA” or “RBA” means the percentage of mean AUCᵢₙₜ of the tested product relative to the mean AUCᵢₙₜ of the reference product for an equal total dose. Unless otherwise specified, relative bioavailability refers to the percentage of the mean AUCᵢₙₜ observed for a full dose of the test product relative to the mean AUCᵢₙₜ observed for two ½-doses of an immediate release liquid solution administered four hours apart for an equal total dose.

“Bioequivalence” means the absence of a significant difference in the rate and extent to which the active ingredient or active moiety in pharmaceutical equivalents or pharmaceutical alternatives become available at the site of drug action when administered at the same molar dose under similar conditions in an appropriately designed study.

When ranges are given by specifying the lower end of a range separately from the upper end of the range, it will be understood that the range may be defined by selectively combining any one of the lower end variables with any one of the upper end variables that is mathematically and physically possible. Thus, for example, if a formulation may contain from 1 to 10 weight parts of a particular ingredient, or 2 to 8 parts of a particular ingredient, it will be understood that the formulation may also contain from 2 to 10 parts of the ingredient. In like manner, if a formulation may contain greater than 1 or 2 weight parts of an ingredient and up to 10 or 9 weight parts of the ingredient, it will be understood that the formulation may contain 1-10 weight parts of the ingredient, 2-9 weight parts of the ingredient, etc. unless otherwise specified, the boundaries of the range (lower and upper ends of the range) are included in the claimed range.

In like manner, when various sub-embodiments of a senior (i.e. principal) embodiment are described herein, it will be understood that the sub-embodiments for the senior embodiment may be combined to define another sub-embodiment. Thus, for example, when a principal embodiment includes sub-embodiments 1, 2 and 3, it will be understood that the principal embodiment may be further limited by any one of sub-embodiments 1, 2 and 3, or any combination of sub-embodiments 1, 2 and 3 that is mathematically and physically possible. In like manner, it will be understood that the principal embodiments described herein may be combined in any manner that is mathematically and physically possible, and that the invention extends to such combinations.

When used herein the term “about” or “substantially” or “approximately” will compensate for variability allowed for in the pharmaceutical industry and inherent in pharmaceutical products, such as differences in product strength due to manufacturing variation and time-induced product degradation. The term allows for any variation which in the practice of pharmaceuticals would allow the product being evaluated to be considered bioequivalent to the recited strength, as described in FDA's March 2003 Guidance for Industry on BIOAVAILABILITY AND BIOEQUIVALENCE STUDIES FOR ORALLY ADMINISTERED DRUG PRODUCTS—GENERAL CONSIDERATIONS.

When used herein the term “gamma-hydroxybutyrate” or GHB, unless otherwise specified, refers to the free base of gamma hydroxy-butyrate, a pharmaceutically acceptable salt of gamma-hydroxybutyric acid, and combinations thereof, their hydrates, solvates, complexes or tautomers forms. Gamma-hydroxybutyric acid salts may be selected from the sodium salt of gamma-hydroxybutyric acid or sodium oxybate, the potassium salt of gamma-hydroxybutyric acid, the magnesium salt of gamma-hydroxybutyric acid, the calcium salt of gamma-hydroxybutyric acid, the lithium salt of gamma-hydroxybutyric, the tetra ammonium salt of gamma-hydroxybutyric acid or any other pharmaceutically acceptable salt forms of gamma-hydroxybutyric acid.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. The term “formulation” or “composition” refers to the quantitative and qualitative characteristics of a drug product or dosage form prepared in accordance with the current invention.

As used herein the doses and strengths of gamma-hydroxybutyrate are expressed in equivalent-gram (g) weights of sodium oxybate unless stated expressly to the contrary. Thus, when considering a dose of gamma-hydroxybutyrate other than the sodium salt of gamma-hydroxybutyrate, one must convert the recited dose or strength from sodium oxybate to the gamma-hydroxybutyrate under evaluation. Thus, if an embodiment is said to provide a 4.5 g dose of gamma-hydroxybutyrate, because the form of gamma-hydroxybutyrate is not specified, it will be understood that the dose encompasses a 4.5 g dose of sodium oxybate, a 5.1 g dose of potassium gamma-hydroxybutyrate (assuming a 126.09 g/mol MW for sodium oxybate and a 142.20 g/mol MW for potassium gamma-hydroxybutyrate), and a 3.7 g dose of the free base (assuming a 126.09 g/mol MW for sodium oxybate and a 104.1 g/mol MW for the free base of gamma-hydroxybutyrate), or by the weight of any mixture of salts of gamma-hydroxybutyric acid that provides the same amount of GHB as 4.5 g of sodium oxybate.

As used herein “microparticle” means any discreet particle of solid material. The particle may be made of a single material or have a complex structure with core and shells and be made of several materials. The terms “microparticle”, “particle”, “microspheres” or “pellet” are interchangeable and have the same meaning. Unless otherwise specified, the microparticle has no particular particle size or diameter and is not limited to particles with volume mean diameter D(4,3) below 1 mm.

As used herein, the “volume mean diameter D(4,3)” is calculated according to the following formula:

$$D ( 4 , 3 ) = \sum ( d 4 i \cdot ni ) / \sum ( d 3 i \cdot ni )$$

wherein the diameter d of a given particle is the diameter of a hard sphere having the same volume as the volume of that particle.

As used herein, the terms “composition”, “oral composition”, “oral pharmaceutical composition”, “finished composition”, “finished formulation” or “formulation” are interchangeable and designate the composition of gamma-hydroxybutyrate comprising modified release microparticles of gamma-hydroxybutyrate, immediate release microparticles of gamma-hydroxybutyrate, and any other excipients. The composition may be described as extended release, delayed release, or modified release.

As used herein, “immediate release” means release of the major part of gamma-hydroxybutyrate over a relatively short period, e.g. at least 75% of the AP is released in 0.75 h, for example, in 30 min.

As used herein, an “immediate release (IR) portion” of a formulation includes physically discreet portions of a formulation, mechanistically discreet portions of a formulation, and pharmacokinetically discreet portions of a formulation that lend to or support a defined IR pharmacokinetic characteristic. Thus, for example, any formulation that releases active ingredient at the rate and extent required of the immediate release portion of the formulations of the present invention includes an “immediate release portion,” even if the immediate release portion is physically integrated in what might otherwise be considered an extended release formulation. Thus, the IR portion may be structurally discreet or structurally indiscreet from (i.e. integrated with) the MR portion. In an embodiment, the IR portion and MR portion are provided as particles, and in other embodiments the IR portion and MR portion are provided as particles discreet from each other.

As used here in, “immediate release formulation” or “immediate release portion” refers to a composition that releases at least 80% of its gamma-hydroxybutyrate in 1 hour when tested in a dissolution apparatus 2 according to USP 38 <711> in a 0.1 N HCl dissolution medium at a temperature of 37° C. and a paddle speed of 75 rpm.

As used herein, “dose dumping” is understood as meaning an immediate and unwanted release of the dose after oral ingestion. In an embodiment, dose dumping may be rapid release of gamma-hydroxybutyrate in the presence of alcohol.

In like manner, a “modified-release (MR) portion” includes that portion of a formulation or dosage form that lends to or supports a particular MR pharmacokinetic characteristic, regardless of the physical formulation in which the MR portion is integrated. The modified release drug delivery systems are designed to deliver drugs at a specific time or over a period of time after administration, or at a specific location in the body. The USP defines a modified release system as one in which the time course or location of drug release or both, are chosen to accomplish objectives of therapeutic effectiveness or convenience not fulfilled by conventional IR dosage forms. More specifically, MR solid oral dosage forms include extended release (ER) and delayed-release (DR) products. A DR product is one that releases a drug all at once at a time other than promptly after administration. Typically, coatings (e.g., enteric coatings) are used to delay the release of the drug substance until the dosage form has passed through the acidic medium of the stomach. An ER product is formulated to make the drug available over an extended period after ingestion, thus allowing a reduction in dosing frequency compared to a drug presented as a conventional dosage form, e.g. a solution or an immediate release dosage form. For oral applications, the term “extended-release” is usually interchangeable with “sustained-release”, “prolonged-release” or “controlled-release”.

Traditionally, extended-release systems provided constant drug release to maintain a steady concentration of drug. For some drugs, however, zero-order delivery may not be optimal and more complex and sophisticated systems have been developed to provide multi-phase delivery. One may distinguish among four categories of oral MR delivery systems: (1) delayed-release using enteric coatings, (2) site-specific or timed release (e.g. for colonic delivery), (3) extended-release (e.g., zero-order, first-order, biphasic release, etc.), and (4), programmed release (e.g., pulsatile, delayed extended release, etc.) See Modified Oral Drug Delivery Systems at page 34 in Gibaldi's DRUG DELIVERY SYSTEMS IN PHARMACEUTICAL CARE, AMERICAN SOCIETY OF HEALTH-SYSTEM PHARMACISTS, 2007 and Rational Design of Oral Modified-release Drug Delivery Systems at page 469 in DEVELOPING SOLID ORAL DOSAGE FORMS: PHARMACEUTICAL THEORY AND PRACTICE, Academic Press, Elsevier, 2009. As used herein, “modified release formulation” or “modified release portion” in one embodiment refers to a composition that releases its gamma-hydroxybutyrate according a multiphase delivery that is comprised in the fourth class of MR products, e.g. delayed extended release. As such it differs from the delayed release products that are classified in the first class of MR products.

As used herein the terms “coating”, “coating layer,” “coating film,” “film coating” and like terms are interchangeable and have the same meaning. The terms refer to the coating applied to a particle comprising the gamma-hydroxybutyrate that controls the modified release of the gamma-hydroxybutyrate.

As used herein, “meal state” or “meal mode” includes the fed state, 2 hours post meal, and the fasted mode. A “fed state” or “fed mode” includes the period of time immediately after consumption of a meal up to two hours post meal. The fed state may include the period less than two hours after eating. A “fasted state” or “fasted mode” includes the period of time after 8 hours post meal consumption. “2 hours post meal” includes the period of time between the fed state and the fasted state. For example, “2 hours post meal” may include the period between at least 2 hours and 8 hours post meal. In all pharmacokinetic testing described herein, unless otherwise stated, the dosage form, or the initial dosage form if the dosing regimen calls for more than one administration, is administered less than two hours after eating, approximately two hours after eating, or more than 8 hours after eating. For example, the dosage form may be administered less than two hours after consumption of a standardized dinner, approximately two hours after consumption of a standardized dinner, or more than 8 hours after consumption of a standardized dinner. The standardized dinner may consist of 25.5% fat, 19.6% protein, and 54.9% carbohydrates.

A “similar PK profile”, a “substantially similar PK profile”, or “comparable bioavailability” means that the mean AUC_{inf} of a test product is from 80% to 125% of the mean AUCᵢₙₜ of a reference product in a suitably designed cross-over trial, the mean plasma concentration at 8 hours (C₈ₕ) of the test product is from 40% to 130% of the mean plasma concentration at 8 hours (C₈ₕ) of the reference product, and/or that the maximum plasma concentration (Cₘₐₓ) of the test product is from 50% to 140% of the Cₘₐₓ of the reference product.

As used herein, “dose proportional” occurs when increases in the administered dose are accompanied by proportional increases in the PK profile, such as the AUC or Cₘₐₓ.

A “fed state PK profile” means the mean AUCᵢₙₜ, the mean plasma concentration at 8 hours (C₈ₕ), and/or the maximum plasma concentration (Cₘₐₓ) of the composition when administered less than two hours after eating.

A “2 hour post meal administration PK profile” means the mean AUCᵢₙₜ, the mean plasma concentration at 8 hours (C₈ₕ), and/or the maximum plasma concentration (Cₘₐₓ) of the composition when administered at least two hours after eating.

Type 1 Narcolepsy (NT1) refers to narcolepsy characterized by excessive daytime sleepiness (“EDS”) and cataplexy. Type 2 Narcolepsy (NT2) refers to narcolepsy characterized by excessive daytime sleepiness without cataplexy. A diagnosis of narcolepsy (with or without cataplexy) may be confirmed by one or a combination of (i) an overnight polysomnogram (PSG) and a Multiple Sleep Latency Test (MSLT) performed within the last 2 years, (ii) a full documentary evidence confirming diagnosis from the PSG and MSLT from a sleep laboratory must be made available, (iii) current symptoms of narcolepsy including: current complaint of EDS for the last 3 months (ESS greater than 10), (iv) mean MWT less than 8 minutes, (v) mean number of cataplexy events of 8 per week on baseline Sleep/Cataplexy Diary, and/or (vi) presence of cataplexy for the last 3 months and 28 events per week during screening period.

As used herein, “adverse events” (AEs) or “treatment emergent adverse events” (TEAEs) means self-reported adverse events by a patient administered a composition for which the AEs are related. AEs or TEAEs may include but not limited to gastrointestinal disorders, nervous system disorders, somnolence, dizziness, nausea, headache, feeling drunk, vomiting, and/or fatigue.

Unless otherwise specified herein, percentages, ratios and numeric values recited herein are based on weight; averages and means are arithmetic means; all pharmacokinetic measurements based on the measurement of bodily fluids are based on plasma concentrations.

It will be understood, when defining a composition by its pharmacokinetic or dissolution properties herein, that the formulation can in the alternative be defined as “means for” achieving the recited pharmacokinetic or dissolution properties. Thus, a formulation in which the modified release portion releases less than 20% of its gamma-hydroxybutyrate at one hour can instead be defined as a formulation comprising “means for” or “modified release means for” releasing less than 20% of its gamma-hydroxybutyrate at one hour. It will be further understood that the structures for achieving the recited pharmacokinetic or dissolution properties are the structures described in the examples hereof that accomplish the recited pharmacokinetic or dissolution properties.

Oral Pharmaceutical Composition for Administration Less than Two Hours after Eating

As the prior art demonstrates, it is extremely difficult to find a formulation that may be administered less than 2 hours after eating a meal and that has pharmacokinetic properties comparable to an immediate release liquid solution of sodium oxybate administered twice nightly taken at least 2 hours after eating.

The inventors have discovered a novel relationship between in vivo gamma-hydroxybutyrate absorption of modified release particles and the effect of food on the absorption of gamma-hydroxybutyrate which permits, for the first time, a composition of gamma-hydroxybutyrate that may be administered less than 2 hours after eating that approximates the bioavailability of a twice-nightly equipotent immediate release liquid solution of sodium oxybate administered at least 2 hours after eating, and that does so across a range of therapeutic doses.

Provided herein is an oral pharmaceutical composition for the treatment of narcolepsy, cataplexy, or excessive daytime sleepiness that includes gamma-hydroxybutyrate in a unit dose suitable for administration less than two hours after eating. In various embodiments, the composition may include gamma-hydroxybutyrate in an extended release formulation, delayed release formulation, or modified release formulation.

Surprisingly, the composition may be administered at any time after eating without being significantly impacted by a food effect. In an embodiment, the composition may be administered less than two hours after eating. For example, the composition may be administered concurrently with food or may be administered immediately after eating, at least 15 minutes after eating, at least 30 minutes after eating, at least 1 hour after eating, at least 1.5 hours after eating, or less than 2 hours after eating.

The composition may provide a substantially similar fed state PK profile and 2 hour post meal administration PK profile. For example, the AUCᵢₙₜ for the composition administered less than two hours after eating may be substantially similar to the AUCᵢₙₜ when the same composition is administered at least two hours after eating. In another example, the Cₘₐₓ for the composition administered less than two hours after eating may be substantially similar to the Cₘₐₓ when the same composition is administered at least two hours after eating.

In an embodiment, when the composition is administered less than two hours after eating, it may achieve a mean AUCᵢₙₜ that is bioequivalent to the mean AUCᵢₙₜ provided by an equal dose of the composition administered at least two hours after eating. In some embodiments, when the composition is administered less than two hours after eating, it may achieve a mean AUCᵢₙₜ that is from 50% to 120%, from 60% to 120%, from 70% to 120%, from 75% to 100%, from 80% to 100%, from 80 to 100%, from 90% to 100%, from 50% to 95%, or from 60% to 90% of the mean AUCᵢₙₜ provided by an equal dose of the composition administered at least two hours after eating.

In an embodiment, when the composition is administered less than two hours after eating, it may achieve a mean Cₘₐₓ that is bioequivalent to the mean Cₘₐₓ provided by an equal dose of the composition administered at least two hours after eating. In some embodiments, when the composition is administered less than two hours after eating, it may achieve a mean Cₘₐₓ that is from 50% to 140%, from 60% to 120%, from 70% to 120%, from 75% to 100%, from 80% to 100%, from 80 to 100%, from 90% to 100%, from 50% to 95%, or from 60% to 90% of the mean Cₘₐₓ provided by an equal dose of the composition administered at least two hours after eating.

The composition may provide a substantially similar fed state PK profile and fasted state PK profile. In an example, the AUCᵢₙₜ for the composition administered less than two hours after eating may be substantially similar to the AUCᵢₙₜ when the same composition is administered in the fasted state. In yet another example, the Cₘₐₓ for the composition administered less than two hours after eating may be substantially similar to the Cₘₐₓ when the same composition is administered in the fasted state.

In an embodiment, when the composition is administered less than two hours after eating, it may achieve a mean AUCᵢₙₜ that is bioequivalent to the mean AUCᵢₙₜ provided by an equal dose of the composition administered in the fasted state. In some embodiments, when the composition is administered less than two hours after eating, it may achieve a mean AUCᵢₙₜ that is from 50% to 120%, from 60% to 120%, from 70% to 120%, from 75% to 100%, from 80% to 100%, from 80 to 100%, from 90% to 100%, from 50% to 95%, or from 60% to 90% of the mean AUCᵢₙₜ provided by an equal dose of the composition administered in the fasted state. In an embodiment, when the composition is administered less than two hours after eating, it may achieve a mean AUCᵢₙₜ with a 90% CI that fall within the 80-125% bioequivalence range of an equal dose of the composition administered in the fasted state with no effect boundaries. In some examples, the composition provides a mean AUCᵢₙₜ when administered less than two hour after eating that is 80%-95% of the mean AUCᵢₙₜ when the composition is administered while fasting. In additional examples, the composition provides a mean AUCᵢₙₜ when administered less than two hour after eating that is 85%-90% of the mean AUCᵢₙₜ when the composition is administered while fasting. In at least one example, a ratio of the AUCₗₐₛₜ (fed) to AUCₗₐₛₜ (fasted) may be about 86 with a 90% CI of 79.9-92.6. In at least one example, a ratio of the AUCᵢₙₜ (fed) to AUCᵢₙₜ (fasted) may be about 86.1 with a 90% CI of 80.0-92.7.

In an embodiment, when the composition is administered less than two hours after eating, it may achieve a mean Cₘₐₓ that is bioequivalent to the mean Cₘₐₓ provided by an equal dose of the composition administered in the fasted state. In some embodiments, when the composition is administered less than two hours after eating, it may achieve a mean Cₘₐₓ that is from 50% to 140%, from 60% to 120%, from 70% to 120%, from 75% to 100%, from 80% to 100%, from 80 to 100%, from 90% to 100%, from 50% to 95%, or from 60% to 90% of the mean Cₘₐₓ provided by an equal dose of the composition administered in the fasted state. In an embodiment, when the composition is administered less than two hours after eating, it may achieve a mean Cₘₐₓ with a 90% CI that fall within the 80-125% bioequivalence range of an equal dose of the composition administered in the fasted state with no effect boundaries. In some examples, the composition provides a mean Cₘₐₓ when administered less than two hours after eating that is 55%-80% of the mean Cₘₐₓ when the composition is administered while fasting. In additional examples, the composition provides a mean Cₘₐₓ when administered less than two hours after eating that is 60%-75% of the mean Cₘₐₓ when the composition is administered while fasting. In at least one example, a ratio of the Cₘₐₓ (fed) to Cₘₐₓ (fasted) may be about 66.6 with a 90% CI of 58.2-76.5.

In an embodiment, the composition provides a Cₘₐₓ that is dose proportional. In additional embodiments, the composition provides no dose dumping.

In an embodiment, compositions of gamma-hydroxybutyrate administered less than two hours after eating may optimize the bioavailability of the gamma-hydroxybutyrate, and roughly approximate the bioavailability of an equal dose of an immediate release liquid solution of sodium oxybate administered twice nightly where the first dose is administered at least two hours after eating.

In some embodiments, the compositions of gamma-hydroxybutyrate administered less than two hours after eating may roughly approximate or exceed the bioavailability of an equal dose of an immediate release solution of sodium oxybate administered twice nightly at least two hours after eating, across the entire therapeutic range of sodium oxybate doses.

In other embodiments, the compositions of gamma-hydroxybutyrate administered less than two hours after eating may produce very little residual drug content in the bloodstream of most patients up to 8 hours after administration but may still be similar to the one observed after administration of an equal dose of an immediate release liquid solution of sodium oxybate administered twice nightly at least two hours after eating.

In an embodiment, there is no significant reduction in safety or efficacy to a patient following administration of the composition. In another embodiment, the compositions of gamma-hydroxybutyrate may improve the therapeutic effectiveness and safety profile of gamma-hydroxybutyrate when administered less than two hours after eating based on novel pharmacokinetic profiles. For example, administration of the gamma-hydroxybutyrate composition less than two hours after eating may result in fewer AEs than administration of the gamma-hydroxybutyrate composition at least two hours after eating or in the fasted state.

In some embodiments, the gamma-hydroxybutyrate composition has a more favorable safety profile as compared to an equal dose of immediate release liquid solution of sodium oxybate administered at t₀ and t₄ₕ in equally divided doses. Most adverse events (AEs) may occur close to Tₘₐₓ, during the Cₘₐₓ period. In an example, the gamma-hydroxybutyrate composition may have only one Tₘₐₓ and one Cₘₐₓ per day due to the once daily administration as compared to multiple Tₘₐₓ and Cₘₐₓ in an immediate release formulation (such as Xyrem®) requiring multiple administrations per day. Therefore, having only one Tₘₐₓ and one Cₘₐₓ by administration of the gamma-hydroxybutyrate composition may result in fewer AEs than administration of the immediate release formulation. In an additional example, the Cₘₐₓ of the gamma-hydroxybutyrate composition may be between the Cₘₐₓ of the first peak and the Cₘₐₓ of the second peak of Xyrem®. The gamma-hydroxybutyrate composition may have a lower Cₘₐₓ than the Cₘₐₓ of an equal dose of an immediate release formulation, such that administration of the gamma-hydroxybutyrate composition may result in fewer AEs than administration of the immediate release formulation. Therefore, administration of the gamma-hydroxybutyrate composition once daily may result in fewer AEs than administration of an equal dose of immediate release liquid solution of sodium oxybate administered at t₀ and t₄ₕ in equally divided doses. In other embodiments, the onset of efficacy of the composition may be earlier than Xyrem® in the fed state.

The gamma-hydroxybutyrate composition may offer a substantial improvement in safety profile to narcolepsy, cataplexy, or excessive daytime sleepiness patients by reducing the amount of adverse events (AEs) over an 8 hour, 12 hour, 16 hour, 20 hour, 24 hour, and/or 48 hour time period following administration of the gamma-hydroxybutyrate composition, as compared to an immediate release sodium oxybate (e.g., Xyrem®) formulation. In particular, the gamma-hydroxybutyrate composition may result in fewer adverse events and fewer Cₘₐₓ periods over an 8 hour, 12 hour, 16 hour, 20 hour, 24 hour, and 48 hour time period, as compared to an immediate release sodium oxybate (e.g., Xyrem®) formulation. Since it appears AEs may be closely related to Cₘₐₓ, by virtue of the gamma-hydroxybutyrate composition having fewer Cₘₐₓ periods than Xyrem®, the gamma-hydroxybutyrate composition may also have fewer AEs over an 8 hour, 12 hour, 16 hour, 20 hour, 24 hour, and/or 48 hour time period, as compared to an immediate release sodium oxybate (e.g., Xyrem®) formulation. Thus, the gamma-hydroxybutyrate composition may have a superior safety profile and/or reduced AEs compared to the prior art (e.g., Xyrem®) or any sodium oxybate composition requiring administration more frequently than once-daily.

In yet another embodiment, the compositions of gamma-hydroxybutyrate administered less than two hours after eating may yield a similar pharmacokinetic profile compared to an immediate release liquid solution of sodium oxybate administered twice nightly at least two hours after eating while potentially giving a reduced dose.

In another embodiment, the compositions of gamma-hydroxybutyrate may allow administration less than two hours after eating compared to the commercial treatment Xyrem®.

In other embodiments, the compositions of gamma-hydroxybutyrate may be administered less than two hours after eating, or may be administered in the fed or fasted state, with improved dissolution and pharmacokinetic profiles compared to an immediate release liquid solution of sodium oxybate administered twice nightly at least two hours after eating.

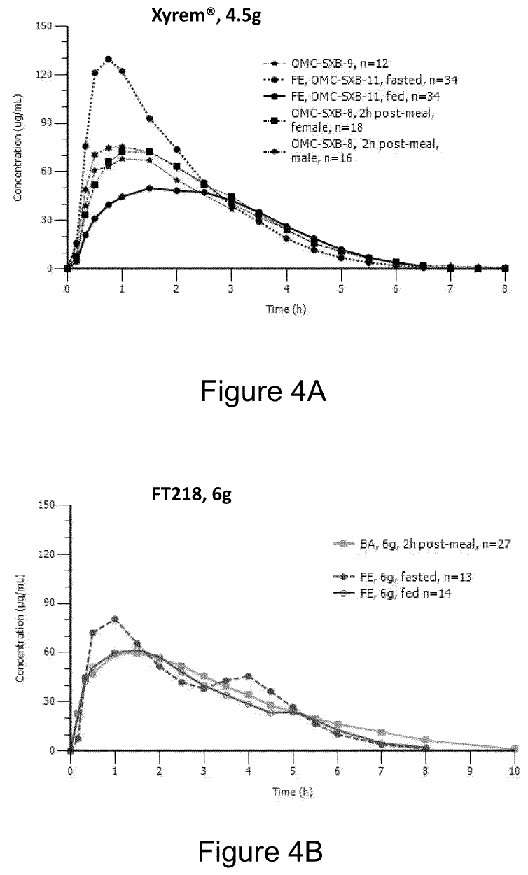

In an embodiment, the composition provides an AUCᵢₙₜ bioequivalent to an AUCᵢₙₜ of Xyrem® as depicted in FIG. 4A, 5A, 5B, 6A, 6B, 7A, or 7B. In another embodiment, the composition provides a Cₘₐₓ that is less than the second Cₘₐₓ of Xyrem® as depicted in FIG. 4A, 5A, 5B, 6A, 6B, 7A, or 7B. In one embodiment, the Cₘₐₓ of the composition when administered less than two hours after eating may be between the first and second Cₘₐₓ of Xyrem®. In yet another embodiment, the composition provides a Cₘₐₓ that is substantially less than the Cₘₐₓ of Xyrem® as depicted in FIG. 4A, 5A, 5B, 6A, 6B, 7A, or 7B. The composition may provide a Cₘₐₓ that is 10-60% less than the Cₘₐₓ of Xyrem® as depicted in FIG. 4A, 5A, 5B, 6A, 6B, 7A, or 7B. The composition may provide a change in Cₘₐₓ between when the composition is administered at least two hours after eating and when the composition is administered less than two hours after eating that is 10-60% less than the change in Cₘₐₓ of Xyrem as depicted in a figure selected from the group consisting of FIGS. 4A, 5A, 5B, 6A, 6B, 7A, and 7B. The composition may provide an AUC that is more dose proportional than the AUC of Xyrem® as depicted in FIG. 4A, 5A, 5B, 6A, 6B, 7A, or 7B.

In particular, a 6 g dose of a composition of gamma-hydroxybutyrate administered less than two hours after eating has been shown to achieve a mean AUCᵢₙₜ of greater than 230 hr*μg/mL, and a mean Cₘₐₓ that is from 50% to 140% of the mean Cₘₐₓ provided by an equal dose of immediate release liquid solution of sodium oxybate administered at t₀ and t₄ₕ in equally divided doses, where the first dose is administered at least two hours after eating a standardized meal. For example, a 6 g dose of the composition administered less than two hours after eating may have a mean AUCᵢₙₜ of about 242 hr*μg/mL and a mean Cₘₐₓ of about 64 μg/mL. In addition, a 9 g dose of a composition of gamma-hydroxybutyrate administered less than two hours after eating may achieve a mean AUCᵢₙₜ of greater than 400 hr*μg/mL, and a mean Cₘₐₓ that may be from 50% to 140% of the mean Cₘₐₓ provided by an equal dose of immediate release liquid solution of sodium oxybate administered at t₀ and t₄ₕ in equally divided doses where the first does is administered at least two hours after a standardized meal. This may be seen by comparing the release profiles and pharmacokinetic profiles in Examples 1-7.

The compositions of gamma-hydroxybutyrate may have both immediate release and modified release portions. The release of gamma-hydroxybutyrate from the immediate release portion is practically uninhibited, and occurs almost immediately in 0.1 N hydrochloric acid dissolution medium. In contrast, while the modified release portion also may release its gamma-hydroxybutyrate almost immediately when fully triggered, the release is not triggered until a predetermined lag-time or the drug is subjected to a suitable dissolution medium such as a phosphate buffer pH 6.8 dissolution medium. Without wishing to be bound by any theory, it is believed that food may have no or low impact on the modified release portion of the composition, as the gamma-hydroxybutyrate from the modified release portion is absorbed in the latter part of the gastro-intestinal tract.

Formulations that achieve this improved bioavailability in the fed state may be described using several different pharmacokinetic parameters. An embodiment of the composition of gamma-hydroxybutyrate includes immediate release and modified release portions, where a 6 g dose of the formulation, when administered less than two hours after eating, may achieve a mean AUCᵢₙₜ of greater than 230, 240, 245, 300, 325, 340, 375, 400, 425, or 450 hr*microgram/mL. In an embodiment, a 6 g does of the composition has a mean AUCᵢₙₜ of greater than 230 hr*microgram/mL. In an embodiment, a 6 g does of the composition has a mean AUCᵢₙₜ of about 242 hr*microgram/mL. For example, when the composition is administered less than two hours after eating, it achieves a mean AUCᵢₙₜ that is from 50% to 120%, from 60% to 120%, from 70% to 120%, from 75% to 100%, from 80% to 100%, from 80 to 100%, from 90% to 100%, from 50% to 95%, or from 60% to 90% of the mean AUCᵢₙₜ provided by an equal dose of the composition administered at least two hours after eating.

An embodiment of the composition of gamma-hydroxybutyrate includes immediate release and modified release portions, where a 6 g dose of the formulation, when administered less than two hours after eating, may achieve a mean Cₘₐₓ of greater than 55, 60, 65, or 70 μg/mL. For example, a 6 g dose of the composition has a mean Cₘₐₓ of about 64 μg/mL. An embodiment of the composition of gamma-hydroxybutyrate includes immediate release and modified release portions, where a 6 g dose of the formulation, when administered less than two hours after eating, may achieve a mean Cₘₐₓ that is from 50% to 140%, from 60% to 140%, from 70% to 140%, from 75% to 135%, from 80% to 135%, from 80 to 130%, from 90% to 110%, from 50% to 95%, or from 60% to 90% of the mean Cₘₐₓ provided by an equal dose of an immediate release liquid solution of sodium oxybate (e.g. Xyrem®) administered at t₀ and t₄ₕ in equally divided doses approximately two hours after a standardized meal. In one embodiment, a 6 g dose of the composition has a mean Cₘₐₓ from 60% to 90%, or from 60% to 140% of the mean Cₘₐₓ provided by an equal dose of an immediate release liquid solution of sodium oxybate (e.g. Xyrem®) administered at t₀ and t₄ₕ in equally divided doses approximately two hours after a standardized meal. In other embodiments, the mean Cₘₐₓ is from 100% to 150% of the of the mean Cₘₐₓ of a first peak of the equal dose of immediate release liquid solution of sodium oxybate administered at t₀ and t₄ₕ in equally divided doses at least two hours after eating and the mean Cₘₐₓ is from 80% to 100% of the of the mean Cₘₐₓ of a second peak of the equal dose of immediate release liquid solution of sodium oxybate administered at t₀ and t₄ₕ in equally divided doses at least two hours after eating.

An embodiment of the composition of gamma-hydroxybutyrate includes immediate release and modified release portions, where a 9 g dose of the formulation, when administered less than two hours after eating, may achieve a mean AUCᵢₙₜ of greater than 350, 400, 450, 500, 525, or 550 hr*microgram/mL. In one embodiment, a 9 g dose of the composition may have a mean AUCᵢₙₜ of greater than 400 hr*microgram/mL.

An embodiment of the composition of gamma-hydroxybutyrate includes immediate release and modified release portions, where a 9 g dose of the formulation, when administered less than 2 hours after eating, may have a mean Cₘₐₓ that is from 50% to 140%, from 60% to 140%, from 70% to 140%, from 75% to 135%, from 80% to 135%, from 80 to 130%, from 90% to 110%, from 50% to 95%, or from 60% to 90% of the mean Cₘₐₓ provided by an equal dose of an immediate release liquid solution of sodium oxybate (e.g. Xyrem®) administered at t₀ and t₄ₕ in equally divided doses approximately two hours after a standardized meal. In one embodiment, a 9 g dose of the composition may have a mean Cₘₐₓ from 60% to 90% or 60% to 120% of the mean Cₘₐₓ provided by an equal dose of an immediate release liquid solution of sodium oxybate (e.g. Xyrem®) administered at t₀ and t₄ₕ in equally divided doses approximately two hours after a standardized meal.

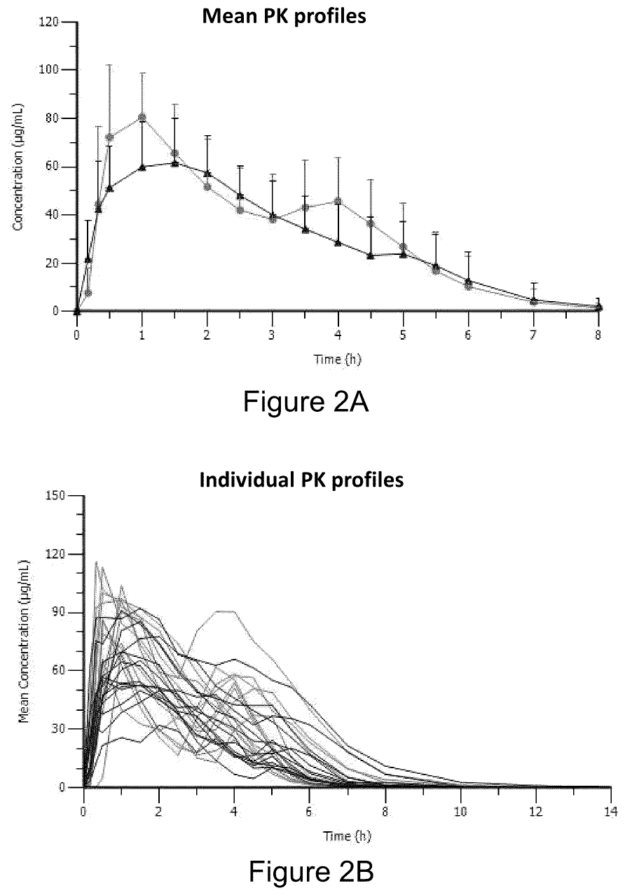

An embodiment of the composition of gamma-hydroxybutyrate yields a plasma concentration versus time curve when administered at a strength of 6 g less than two hours after a standardized evening meal substantially as depicted in FIG. 2A.

Another embodiment of the composition of gamma-hydroxybutyrate yields a plasma concentration versus time curve when administered once nightly at a strength of 6 g less than two hours after eating substantially as depicted in FIG. 4B.

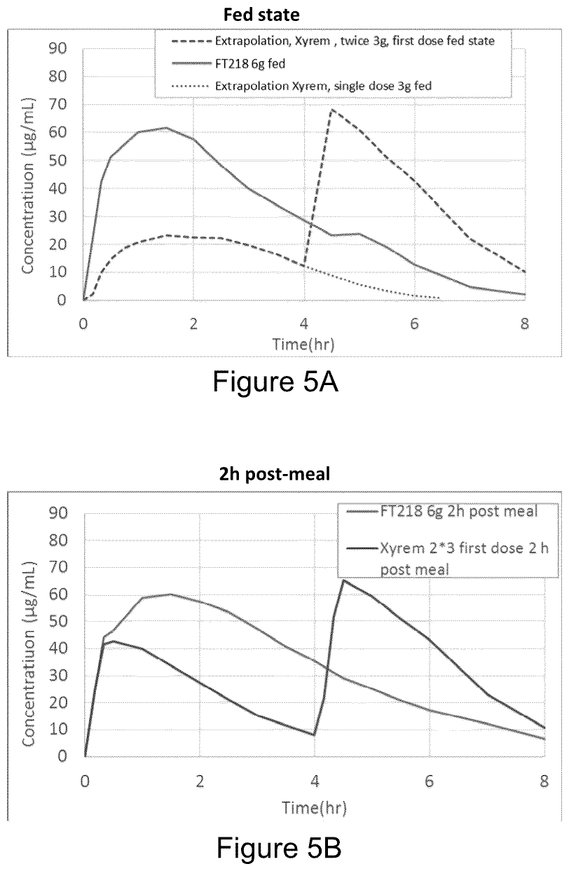

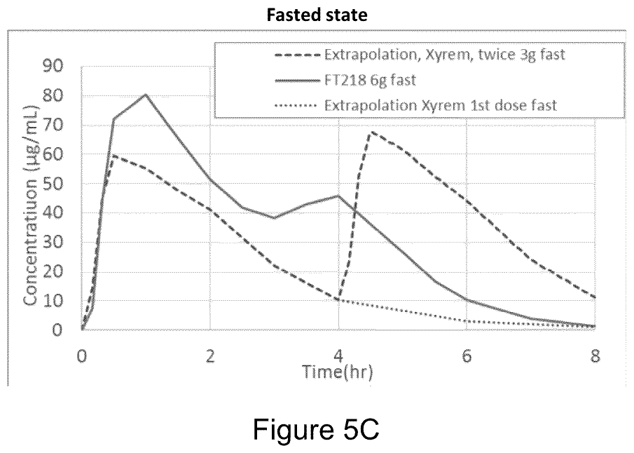

Yet another embodiment of the composition of gamma-hydroxybutyrate yields a plasma concentration versus time curve when administered once nightly at a strength of 6 g less than two hours after eating substantially as depicted in FIG. 5A.

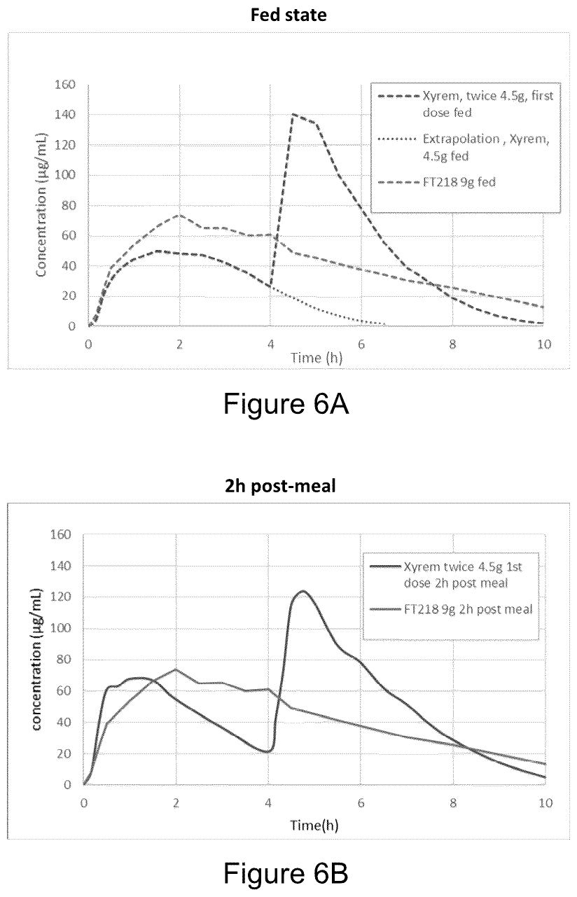

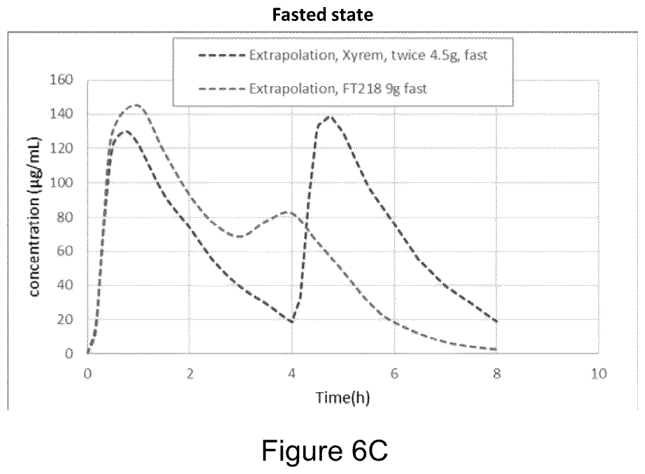

An embodiment of the composition of gamma-hydroxybutyrate yields a plasma concentration versus time curve when administered once nightly at a strength of 9 g less than two hours after eating substantially as depicted in FIG. 6A.

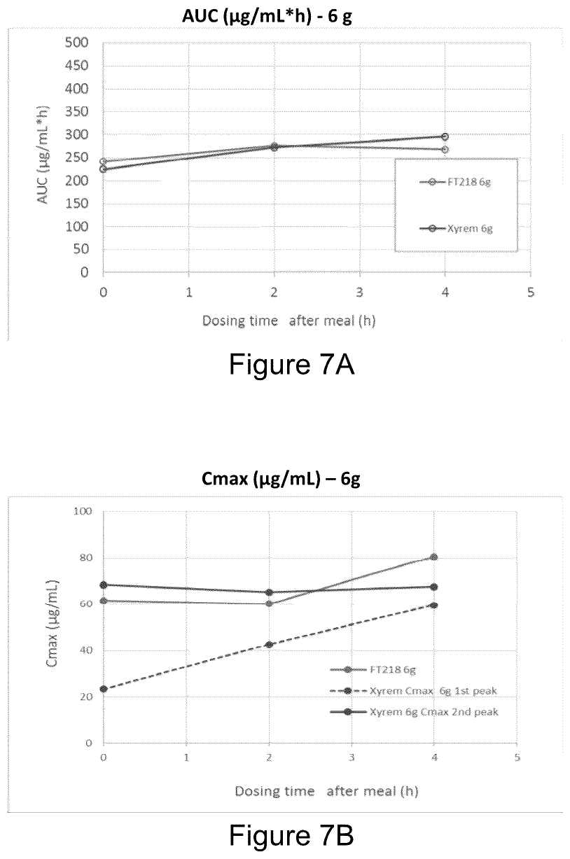

Another embodiment of the composition of gamma-hydroxybutyrate yields an AUC profile when administered once nightly at a strength of 6 g between 0 and 4 hours after eating substantially as depicted in FIG. 7A.

Yet another embodiment of the composition of gamma-hydroxybutyrate yields a Cₘₐₓ profile when administered once nightly at a strength of 6 g between 0 and 4 hours after a standardized evening meal substantially as depicted in FIG. 7B.

In any of these embodiments, the formulation may be effective to treat narcolepsy Type 1 or Type 2. The treatment of narcolepsy is defined as reducing excessive daytime sleepiness or reducing the frequency of cataplectic attacks. In various embodiments, the composition is sufficient to be administered once daily. For example, the composition may be sufficient to administer in the morning or at night less than 2 hours after eating a meal. The formulation is also effective to induce sleep for at least 6 to 8 consecutive hours. In one embodiment, the composition administered less than two hours after eating is effective to induce sleep for at least 8 consecutive hours. In various embodiments, the formulation is effective to induce sleep for at least 6 hours, at least 7 hours, at least 8 hours, at least 9 hours, or at least 10 hours. In other embodiments, the formulation is effective to induce sleep for up to 6 hours, up to 7 hours, up to 8 hours, up to 9 hours, or up to 10 hours.

In any of these embodiments, the composition may include immediate release and modified release portions, where the modified release portion includes gamma hydroxybutyrate particles coated by a polymer carrying free carboxylic groups and a hydrophobic compound having a melting point equal or greater than 40° C., and the ratio of gamma-hydroxybutyrate in the immediate release portion and the modified release portion is from 10/90 to 65/35. The polymers comprising free carboxylic groups may have a pH dissolution trigger of from 5.5 to 6.97 and may be methacrylic acid copolymers having a pH dissolution trigger of from 5.5 to 6.97.

### Structural Embodiments

Various techniques are known for formulating modified release dosage forms including, for example, the techniques described in U.S. Pat. No. 8,101,209 to Legrand et al. (“Legrand”). Legrand provides a system ensuring that the active ingredient is released with certainty from the modified release dosage form by means of a dual mechanism of “time-dependent” and “pH-dependent” release. Legrand did not describe any dosage forms for delivering sodium oxybate or other forms of gamma-hydroxybutyrate administered less than two hours after eating.

In various embodiments, the composition of any of the embodiments herein may be administered at any meal state. In particular, the composition may be administered in the fed state, at least two hours after eating, or in the fasted state. In one embodiment, the composition is administered in the fed state, which is less than two hours after eating. For example, the composition may be administered while eating, immediately after eating, up to 30 minutes after eating, at least 30 minutes after eating, up to 45 minutes after eating, up to 1 hour after eating, up to 1.25 hours after eating, up to 1.5 hours after eating, up to 1.75 hours after eating, up to 1.8 hours after eating, up to 1.9 hours after eating, or up to 1.95 hours after eating. In some embodiments, the composition may be administered less than two hours after eating a standardized evening meal.

In an embodiment, the composition of gamma-hydroxybutyrate may include immediate release and modified release portions, wherein: (a) the modified release portion comprises coated particles of gamma-hydroxybutyrate; (b) the coating comprises a polymer carrying free carboxylic groups and a hydrophobic compound having a melting point equal or greater than 40° C.; and (c) the ratio of gamma-hydroxybutyrate in the immediate release portion and the modified release portion is from 10/90 to 65/35.

In an embodiment, the composition of gamma-hydroxybutyrate may include immediate release and modified release portions, a suspending or viscosifying agent, and an acidifying agent, wherein: (a) the modified release portion comprises coated particles of gamma-hydroxybutyrate; (b) the coating comprises a polymer carrying free carboxylic groups and a hydrophobic compound having a melting point equal or greater than 40° C.; and (c) the ratio of gamma-hydroxybutyrate in the immediate release portion and the modified release portion is from 10/90 to 65/35.

In an embodiment, the composition of gamma-hydroxybutyrate may include immediate release and modified release portions, wherein: (a) the modified release portion comprises coated particles of gamma-hydroxybutyrate; (b) the coating comprises a polymer carrying free carboxylic groups and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35; and (e) the coating is from 10 to 50% of the weight of the particles.

In an embodiment, the composition of gamma-hydroxybutyrate may include immediate release and modified release portions, wherein: (a) the modified release portion comprises coated particles of gamma-hydroxybutyrate; (b) the coating comprises a polymer carrying free carboxylic groups having a pH trigger of from 5.5 to 6.97 and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35; and (e) the coating is from 10 to 50% of the weight of the particles.

In an embodiment, the composition of gamma-hydroxybutyrate may include immediate release and modified release portions, wherein: (a) the modified release portion comprises coated particles of gamma-hydroxybutyrate; (b) the coating comprises a methacrylic acid copolymer carrying free carboxylic groups having a pH trigger of from 5.5 to 6.97 and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35; and (e) the coating is from 10 to 50% of the weight of the particles.

In various embodiments, gamma-hydroxybutyrate may be present in the composition as a 4.5 g, 6.0 g, 7.5 g, or 9.0 g dose. In some embodiments, the dosage of gamma-hydroxybutyrate may be sufficient to administer the composition once daily.

### Pharmacokinetics

As mentioned in the definitions section of this document, each of the sub-embodiments may be used to further characterize and limit each of the foregoing principal embodiments. In addition, more than one of the following sub-embodiments may be combined and used to further characterize and limit each of the foregoing principal embodiments, in any manner that is mathematically and physically possible.

In an embodiment, the oral pharmaceutical composition of gamma-hydroxybutyrate may be in a unit dose suitable for administration less than two hours after eating for the treatment of narcolepsy, cataplexy, or excessive daytime sleepiness. Without being limited to a particular theory, the composition may include a modified release portion that delays release of a portion of the gamma-hydroxybutyrate until the composition reaches the small intestine, such that ingestion of food has a limited effect on the absorption of the gamma-hydroxybutyrate.

In an embodiment, the oral pharmaceutical composition of gamma-hydroxybutyrate may be in a unit dose suitable for administration once daily for the treatment of narcolepsy, cataplexy, or excessive daytime sleepiness. In an example, the composition may exhibit rapid initial absorption comparable to twice-nightly IR sodium oxybate. In another example, the composition may demonstrate a lower overall Cₘₐₓ than twice-nightly IR sodium oxybate. In other examples, the composition may provide mean blood concentrations (ug/ml) at 8 hours similar to that of twice-nightly IR sodium oxybate.

In various embodiments, the composition provides a substantially similar fed state PK profile and 2 hour post meal administration PK profile. In some embodiments, a 4.5 g, 7.5 g, and/or 9 g dose may exhibit a PK profile consistent with those desired for once-nightly dosing. In various embodiments, the composition exhibits pharmacokinetics that are dose proportional when administered once daily, 2 hours post meal. In an embodiment, the composition provides a Cₘₐₓ that is dose proportional. For example, the composition provides a Cₘₐₓ that is dose proportional across once daily doses of 4.5 g, 7.5 g, and 9 g. In an embodiment, the composition provides an AUC that is dose proportional. For example, the Cₘₐₓ of a 6 g dose is proportional to the Cₘₐₓ of a 9 g dose of the composition. In another embodiment, the composition is dose proportional by a factor of about 1 to about 1.3. In an example, the Cₘₐₓ may be dose proportional by a factor of about 1. In another example, the AUC may be dose proportional by a factor of about 1.3. In some examples, the increase in the AUC may be slightly more than proportional. The composition may exhibit predictable increases in plasma levels with increasing doses, consistent with the PK profile desired for a once-nightly sodium oxybate formulation.

In an embodiment, the once-nightly controlled-release sodium oxybate composition may demonstrate lower overall Cₘₐₓ and similar total AUC, compared to twice-nightly sodium oxybate. In an embodiment, the once-nightly composition safety profile may be consistent with what is known for sodium oxybate.

In an embodiment, the composition provides no dose dumping. For example, the ingestion of alcohol does not result in an immediate release of gamma-hydroxybutyrate.

In another embodiment, there is no significant reduction in safety or efficacy to a patient following administration of the composition less than two hours after eating. In some embodiments, the safety is improved over the administration of Xyrem®, such that the Cₘₐₓ of the composition is less than the Cₘₐₓ after administration of the second dose of Xyrem® when administered in the fed state. In other embodiments, the composition is more effective than Xyrem® when administered in the fed state. For example, the Cₘₐₓ of the composition is higher than the Cₘₐₓ after administration of the first dose of Xyrem® when administered in the fed state.

In an embodiment, the composition provides an AUCᵢₙₜ bioequivalent to an AUCᵢₙₜ of Xyrem® as depicted in a figure selected from FIGS. 4A, 5A, 5B, 6A, 6B, and 7A. In an embodiment, the composition provides a Cₘₐₓ that is less than the Cₘₐₓ of Xyrem® as depicted in a figure selected from FIGS. 4A, 5A, 5B, 6A, 6B, 7A, and 7B. In an embodiment, the composition provides a Cₘₐₓ that is substantially less than the Cₘₐₓ of Xyrem® as depicted in a figure selected from FIGS. 4A, 5A, 5B, 6A, 6B, and 7B. In an embodiment, the composition provides a Cₘₐₓ that is 10-60% less than the Cₘₐₓ of Xyrem® as depicted in a figure selected from FIGS. 4A, 5A, 5B, 6A, 6B, and 7B. In an embodiment, the composition provides an AUC that is more dose proportional than the AUC of Xyrem® as depicted in a figure selected from FIGS. 4A, 5A, 5B, 6A, 6B, and 7A.

In various embodiments, a 6 g dose of the composition of gamma-hydroxybutyrate may be characterized as having been shown to achieve a mean AUCᵢₙₜ of greater than 230, 240, 245, 250, 255, 260, 275, 300, 325, or 350 hr*microgram/mL when administered once less than two hours after eating. An upper limit on mean AUCᵢₙₜ for such 6 g dose may be set at 300 or 350 hr*μg/mL. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In additional embodiments, a 6 g dose of the composition of gamma-hydroxybutyrate may be characterized as having been shown to achieve a mean Cₘₐₓ of greater than 50, 55, 60, 65, 70, 75, 80, 85, or 90 μg/mL when administered once less than two hours after eating. An upper limit on mean Cₘₐₓ for such 6 g dose may be set at 75 or 90 μg/mL. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In additional embodiments, a 6 g dose of the composition of gamma-hydroxybutyrate administered less than two hours after eating may be characterized as having been shown to achieve a mean Cₘₐₓ that is from 50% to 140%, from 60% to 140%, from 70 to 140%, from 75% to 135%, from 80% to 135%, or from 80 to 130% of the mean Cₘₐₓ provided by an equal dose of immediate release liquid solution of gamma-hydroxybutyrate administered at t₀ and t₄ₕ in two equally divided doses administered at least two hours after a standardized meal. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In various embodiments, a 6 g dose of the composition of gamma-hydroxybutyrate may be characterized as having been shown to achieve a mean AUC₈ₕ of greater than 1, 2, 3, 4, 5, or 6 μg/mL when administered once less than two hours after eating. An upper limit on mean AUC₈ₕ for such 6 g dose may be set at 5 or 6 μg/mL. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In various embodiments, a 6 g dose of the composition of gamma-hydroxybutyrate may be characterized as having been shown to achieve a mean AUC₀₋₈ of greater than 150, 175, 200, 225, 250, 300, or 350 hr*μg/mL when administered once less than two hours after eating. An upper limit on mean AUC₀₋₈ for such 6 g dose may be set at 300 or 350 hr*μg/mL. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In various embodiments, a 6 g dose of the composition of gamma-hydroxybutyrate may be characterized as having been shown to achieve a mean AUC₀₋ₜ of greater than 150, 175, 200, 225, 250, 300, or 350 hr*μg/mL when administered once less than two hours after eating. An upper limit on mean AUC₀₋ₜ for such 6 g dose may be set at 300 or 350 hr*μg/mL. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In various embodiments, a 6 g dose of the composition of gamma-hydroxybutyrate may be characterized as having been shown to achieve a mean % AUC, ext of greater than 0.1%, 0.2%, or 0.3% when administered once less than two hours after eating. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In various embodiments, a 6 g dose of the composition of gamma-hydroxybutyrate may be characterized as having been shown to achieve a tₘₐₓ of greater than 0.5, 1, 1.5, 2, or 2.5 hours when administered once less than two hours after eating. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In various embodiments, a 6 g dose of the composition of gamma-hydroxybutyrate may be characterized as having been shown to achieve a mean t_{1/2} of greater than 0.5, 1, or 1.5 hours when administered once less than two hours after eating. In these embodiments, the 6 g dose of the composition may be administered less than two hours after eating a meal. For example, the 6 g dose of the composition may be administered once daily, in the morning or the evening. In other embodiments, the 6 g dose of the composition may be administered in the fed state.

In one embodiment, a 6 g dose of the composition administered less than two hours after eating may achieve a mean AUC_{inf} of greater than 230 hr·μg/mL, and a mean Cₘₐₓ that is from 50% to 140% of the mean Cₘₐₓ provided by an equal dose of immediate release liquid solution of sodium oxybate administered at t₀ and t₄ₕ in equally divided doses at least two hours after eating.

In various embodiments, a 9 g dose of the composition of gamma-hydroxybutyrate administered less than two hours after eating may be characterized as having been shown to achieve a mean AUCᵢₙₜ of greater than 350, 400, 450, 460, 470, 480, 490, 500, 525, 550, 575, or 600 hr*μg/mL. An upper limit on mean AUCᵢₙₜ for such 9 g dose may be set at 550 or 600 hr*microgram/mL. In these embodiments, the 9 g dose of the composition may be administered less than two hours after eating a meal. For example, the 9 g dose of the composition may be administered once daily, in the morning or the evening. In other embodiments, the 9 g dose of the composition may be administered in the fed state.

In additional embodiments, a 9 g dose of the composition of gamma-hydroxybutyrate administered less than two hours after eating may be characterized as having a mean Cₘₐₓ of greater than 60, 65, 70, 75, 80, 85, 90, 95, or 100 μg/mL. An upper limit on mean Cₘₐₓ for such 9 g dose may be set at 125 or 100 μg/mL. In these embodiments, the 9 g dose of the composition may be administered less than two hours after eating a meal. For example, the 9 g dose of the composition may be administered once daily, in the morning or the evening. In other embodiments, the 9 g dose of the composition may be administered in the fed state.

In additional embodiments, a 9 g dose of the composition of gamma-hydroxybutyrate administered less than two hours after eating may be characterized as having a mean Cₘₐₓ that is from 50% to 120% or from 60% to 120% of the mean Cₘₐₓ provided by an equal dose of immediate release liquid solution of gamma-hydroxybutyrate administered at t₀ and t₄ₕ in two equally divided doses administered at least two hours after a standardized meal. In these embodiments, the 9 g dose of the composition may be administered less than two hours after eating a meal. For example, the 9 g dose of the composition may be administered once daily, in the morning or the evening. In other embodiments, the 9 g dose of the composition may be administered in the fed state.

In one embodiment, a 9 g dose of the composition administered less than two hours after eating may achieve a mean AUCᵢₙₜ of greater than 500 hr·μg/mL, and a mean Cₘₐₓ that is from 50% to 120% of the mean Cmax provided by an equal dose of immediate release liquid solution of sodium oxybate administered at t₀ and t₄ₕ in equally divided doses at least two hours after a standardized evening meal.

Still further embodiments may be defined based on a pharmacokinetic comparison of the composition of gamma-hydroxybutyrate to an immediate release solution of gamma-hydroxybutyrate. Therefore, in additional embodiments, the composition of gamma-hydroxybutyrate administered less than two hours after eating may achieve a relative bioavailability (RBA) of greater than 80%, 85%, 90%, or 95% when compared to an equal dose of an immediate release liquid solution of sodium oxybate administered at t₀ and t₄ₕ in equally divided doses administered at least two hours after a standardized meal. For example, a 6 g and 9 g dose of the formulation administered less than two hours after eating may have an RBA of greater than 80%, 85% or 90% when compared to an equal dose of an immediate release liquid solution of sodium oxybate administered at t₀ and t₄ₕ in equally divided doses administered at least two hours after a standardized meal

The compositions of gamma-hydroxybutyrate may also be defined by the concentration/time curves that they produce when tested according to the Examples. Therefore, in other embodiments, a 6 g or 9 g dose of the composition of gamma-hydroxybutyrate administered less than two hours after eating may achieve a time/concentration curve substantially as shown in FIGS. 5A and 6A respectively herein. In other embodiments, the composition may achieve a time/concentration curve substantially as shown in FIG. 2A, 3B, or 4B herein.

The compositions of gamma-hydroxybutyrate may also be defined based on the time required to reach maximum blood concentration of gamma-hydroxybutyrate. Thus, in additional embodiments, the composition of gamma-hydroxybutyrate may achieve a mean Tₘₐₓ of 0.5 to 2.5 hours. In various embodiments, the composition of gamma-hydroxybutyrate may achieve a mean Tₘₐₓ of about 0.5, 0.75, 1.0, 1.25, 1.5, 1.75, 2.0, 2.25, or 2.5 hours when administered less than two hours after eating. A lower limit on the median Tₘₐₓ in any of the foregoing ranges can alternatively be set at 0.5 or 1.0 hours.

In another embodiment, a 6 g dose of the composition may achieve a mean AUCᵢₙₜ of greater than 230 hr·μg/mL and a mean Cₘₐₓ of greater than 60 microgram/mL when administered less than two hours after eating.

In still another embodiment, a 9 g dose of the formulation may achieve a mean AUCᵢₙₜ of greater than 400 hr·μg/mL and a mean Cₘₐₓ of greater than 60 microgram/mL when administered less than two hours after eating.

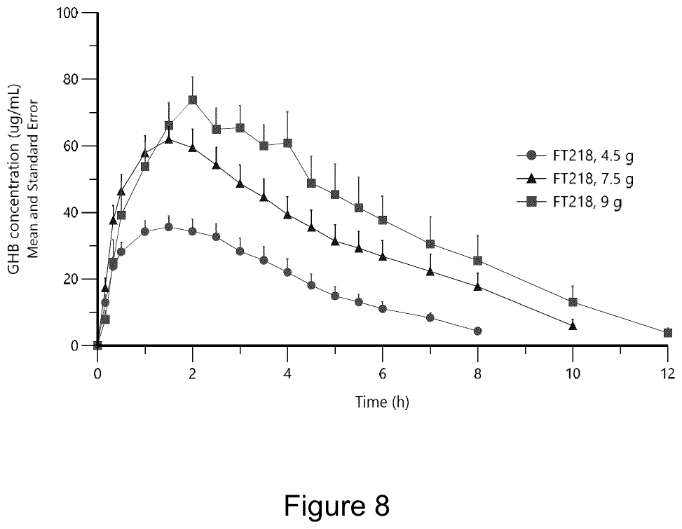

In an embodiment, 4.5 g, 7.5 g and 9 g doses of the composition may exhibit similar overall mean pharmacokinetics profiles when administered once daily, 2 hours after eating. In some embodiments, 4.5 g, 7.5 g and 9 g doses of the composition may exhibit mean pharmacokinetics profiles as shown in FIG. 8 when administered once daily, 2 hours after eating. In an example, 4.5 g, 7.5 g and 9 g doses of the composition may exhibit a median Tₘₐₓ between 1.5 and 2 hours (FIG. 8). In an example, 4.5 g, 7.5 g and 9 g doses of the composition may achieve a mean Cₘₐₓ from 42.9 to 84.5 μg/mL across the increasing doses. Following Cₘₐₓ, blood levels may gradually decrease overnight. In an example, the composition may achieve a mean AUCᵢₙₜ of 191, 358 and 443 μg·h/mL for the 4.5, 7.5 and 9 g doses respectively when administered once daily, 2 hours after eating. In an example, the composition may exhibit mean concentrations at 8 hours of 4.8, 19.7 and 25.5 μg/mL for the 4.5, 7.5 and 9 g doses respectively when administered once daily, 2 hours after eating.

### Structural Embodiments

The compositions of gamma-hydroxybutyrate may be provided in any dosage form that is suitable for oral administration, including tablets, capsules, liquids, orally dissolving tablets, and the like. In one embodiment, they are provided as dry particulate formulations (i.e. granules, powders, coated particles, microparticles, pellets, microspheres, etc.), in a sachet or other suitable discreet packaging units. A particulate formulation will be mixed with tap water shortly before administration. In one embodiment, the composition may be mixed with 50 mL water prior to administration. In another embodiment, the composition is an oral pharmaceutical composition.

In various embodiments, the composition includes gamma-hydroxybutyrate present in a unit dose of at least 4.5 g, at least 6.0 g, at least 7.5 g, or at least 9.0 g. In various embodiments, the composition includes gamma-hydroxybutyrate present in a unit dose of more than 4.5 g, more than 6.0 g, more than 7.5 g, or more than 9.0 g. In one embodiment, the formulation includes 6 g gamma-hydroxybutyrate. In another embodiment, the formulation includes 9 g gamma-hydroxybutyrate.

In one embodiment, the formulation comprises immediate release and modified release portions, wherein: (a) the modified release portion comprises coated microparticles of gamma-hydroxybutyrate; and (b) the ratio of gamma-hydroxybutyrate in the immediate release portion and the modified release portion is from 10/90 to 65/35.

In one embodiment, the formulation comprises immediate release and modified release portions, wherein: (a) the modified release portion comprises coated microparticles of gamma-hydroxybutyrate; and (b) the ratio of gamma-hydroxybutyrate in the immediate release portion and the modified release portion is from 40/60 to 60/40.

In another embodiment, the formulation comprises immediate release and modified release portions, wherein: (a) the modified release portion comprises coated microparticles of gamma-hydroxybutyrate; (b) the coating of said modified release particles of gamma-hydroxybutyrate comprises a polymer carrying free carboxylic groups and a hydrophobic compound having a melting point equal or greater than 40° C.; and (c) the ratio of gamma-hydroxybutyrate in the immediate release portion and the modified release portion is from 10/90 to 65/35 or 40/60 to 60/40.

In another embodiment, the formulation comprises immediate release and modified release portions, wherein: (a) the modified release portion comprises coated microparticles of gamma-hydroxybutyrate; (b) the coating of said modified release particles of gamma-hydroxybutyrate comprises a polymer carrying free carboxylic groups and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35 or 40/60 to 60/40; and (e) the film coating is from 10 to 50% of the weight of the microparticles.

In another embodiment the formulation comprises immediate release and modified release portions, wherein: (a) the modified release portion comprises coated particles of gamma-hydroxybutyrate; (b) the coating of said modified release particles of gamma-hydroxybutyrate comprises a polymer carrying free carboxylic groups having a pH trigger of from 5.5 to 6.97 and a hydrophobic compound having a melting point equal or greater than 40° C.; (c) the weight ratio of the hydrophobic compound to the polymer carrying free carboxylic groups is from 0.4 to 4; (d) the ratio of gamma-hydroxybutyrate in the immediate release portion and the modified release portion is from 10/90 to 65/35 or 40/60 to 60/40; and (e) the coating is from 10 to 50% of the weight of the particles.

In an embodiment, the polymer carrying free carboxylic groups comprises from 100% poly (methacrylic acid, ethyl acrylate) 1:1 and 0% poly (methacrylic acid, methylmethacrylate) 1:2 to 2% poly (methacrylic acid, ethyl acrylate) 1:1 and 98% poly (methacrylic acid, methylmethacrylate) 1:2; and the hydrophobic compound comprises hydrogenated vegetable oil.

In an embodiment, the formulation includes excipients to improve the viscosity and the pourability of the mixture of the particulate formulation with tap water. As such, the particulate formulation comprises, besides the immediate release and modified release particles of gamma-hydroxybutyrate, one or more suspending or viscosifying agents or lubricants.

Suspending or viscosifying agents may be chosen from the group consisting of xanthan gum, medium viscosity sodium carboxymethyl cellulose, mixtures of microcrystalline cellulose and sodium carboxymethyl cellulose, mixtures of microcrystalline cellulose and guar gum, medium viscosity hydroxyethyl cellulose, agar, sodium alginate, mixtures of sodium alginate and calcium alginate, gellan gum, carrageenan gum grade iota, kappa or lambda, and medium viscosity hydroxypropylmethyl cellulose.

Medium viscosity sodium carboxymethyl cellulose corresponds to grade of sodium carboxymethyl cellulose whose viscosity, for a 2% solution in water at 25° C., is greater than 200 mPa·s and lower than 3100 mPa·s.

Medium viscosity hydroxyethyl cellulose corresponds to a grade of hydroxyethyl cellulose whose viscosity, for a 2% solution in water at 25° C., is greater than 250 mPa·s and lower than 6500 mPa·s. Medium viscosity hydroxypropylmethyl cellulose corresponds to a grade of hydroxypropylmethyl cellulose whose viscosity, for a 2% solution in water at 20° C., is greater than 80 mPa·s. and lower than 3800 mPa·s.

In one embodiment, the suspending or viscosifying agents are xanthan gum, especially Xantural 75™ from Kelco, hydroxyethylcellulose, especially Natrosol 250M™ from Ashland, Kappa carrageenan gum, especially Gelcarin PH812™ from FMC Biopolymer, and lambda carrageenan gum, especially Viscarin PH209™ from FMC Biopolymer.

In an embodiment, the composition of gamma-hydroxybutyrate comprises from 1 to 15% of viscosifying or suspending agents. In other embodiments, the composition of gamma-hydroxybutyrate comprises viscosifying or suspending agents in an amount from 2 to 10%, from 2 to 5%, or from 2 to 3% of the formulation.

In an embodiment, the composition of gamma-hydroxybutyrate is in the form of a powder that is intended to be dispersed in water prior to administration and further comprises from 1 to 15% of a suspending or viscosifying agent selected from a mixture of xanthan gum, carrageenan gum and hydroxyethylcellulose or xanthan gum and carrageenan gum.

In an embodiment, the composition of gamma-hydroxybutyrate is in the form of a powder that is intended to be dispersed in water prior to administration and further comprises: from 1.2 to 15% of an acidifying agent selected from malic acid and tartaric acid; and from 1 to 15% of a suspending or viscosifying agent selected from a mixture of xanthan gum, carrageenan gum and hydroxyethylcellulose or xanthan gum and carrageenan gum.

In one embodiment, the composition of gamma-hydroxybutyrate comprises about 1% of lambda carrageenan gum or Viscarin PH209™, about 1% of medium viscosity grade of hydroxyethyl cellulose or Natrosol 250M™, and about 0.7% of xanthan gum or Xantural 75™. For a 4.5 g dose unit, these percentages will typically equate to about 50 mg xanthan gum (Xantural 75™), about 75 mg carrageenan gum (Viscarin PH209™), and about 75 mg hydroxyethylcellulose (Natrasol 250M™).

Alternative packages of viscosifying or suspending agents, for a 4.5 g dose, include about 50 mg xanthan gum (Xantural 75™) and about 100 mg carrageenan gum (Gelcarin PH812™), or about 50 mg xanthan gum (Xantural 75™), about 75 mg hydroxyethylcellulose (Natrasol 250M™), and about 75 mg carrageenan gum (Viscarin PH109™).

In an embodiment, the composition of gamma-hydroxybutyrate further comprises a lubricant or a glidant, besides the immediate release and modified release particles of gamma-hydroxybutyrate. In various embodiments, the lubricants and glidants are chosen from the group consisting of salts of stearic acid, in particular magnesium stearate, calcium stearate or zinc stearate, esters of stearic acid, in particular glyceryl monostearate or glyceryl palmitostearate, stearic acid, glycerol behenate, sodium stearyl fumarate, talc, and colloidal silicon dioxide. In one embodiment, the lubricant or glidant is magnesium stearate. The lubricant or glidant may be used in the particulate formulation in an amount of from 0.1 to 5%. In one embodiment, the amount of lubricant or glidant is about 0.5%. For example, the composition of gamma-hydroxybutyrate may include about 0.5% of magnesium stearate.

A composition of gamma-hydroxybutyrate may further include an acidifying agent. The acidifying agent helps to ensure that the release profile of the formulation in 0.1 N HCl will remain substantially unchanged for at least 15 minutes after mixing, which is approximately the maximum length of time a patient might require before consuming the dose after mixing the formulation with tap water.

In one embodiment, the formulation is a powder, and further comprising an acidifying agent and a suspending or viscosifying agent in the weight percentages recited herein.

The acidifying agents may be chosen from the group consisting of malic acid, citric acid, tartaric acid, adipic acid, boric acid, maleic acid, phosphoric acid, ascorbic acid, oleic acid, capric acid, caprylic acid, and benzoic acid. In various embodiments, the acidifying agent is present in the formulation from 1.2 to 15%, from 1.2 to 10%, or from 1.2 to 5%. In one embodiment, the acidifying agents are tartaric acid and malic acid. In another embodiment, the acidifying agent is malic acid.

When tartaric acid is employed, it may be employed in an amount of from 1 to 10%, from 2.5 to 7.5%, or about 5%. In various embodiments, the amount of malic acid in the composition of gamma-hydroxybutyrate is from 1.2 to 15%, from 1.2 to 10%, from 1.2 to 5%, or from 1.6% or 3.2%. In one embodiment, the amount of malic acid in the composition of gamma hydroxybutyrate is about 1.6%.

The composition of gamma-hydroxybutyrate includes an immediate release portion and a modified release portion of gamma-hydroxybutyrate, and in an embodiment, the formulation is a particulate formulation that includes a plurality of immediate release gamma-hydroxybutyrate particles and a plurality of modified release gamma-hydroxybutyrate particles. The molar ratio of gamma-hydroxybutyrate in the immediate release and modified release portions ranges from 0.11:1 to 1.86:1, from 0.17:1 to 1.5:1, from 0.25:1 to 1.22:1, from 0.33:1 to 1.22:1, from 0.42:1 to 1.22:1, from 0.53:1 to 1.22:1, from 0.66:1 to 1.22:1, from 0.66:1 to 1.5:1, from 0.8:1 to 1.22:1. In one embodiment, the molar ratio of gamma-hydroxybutyrate in the immediate release and modified release portions is about 1:1. The molar percentage of gamma-hydroxybutyrate in the immediate release portion relative to the total of gamma-hydroxybutyrate in the formulation ranges from 10% to 65%, from 15 to 60%, from 20 to 55%, from 25 to 55%, from 30 to 55%, from 35 to 55%, from 40 to 55%, from 40 to 60%, or from 45 to 55%. In one embodiment, the molar percentage of gamma-hydroxybutyrate in the immediate release portion relative to the total of gamma-hydroxybutyrate in the formulation ranges from 40% to 60%. In an embodiment, the molar percentage of the gamma-hydroxybutyrate in the immediate release portion relative to the total of gamma-hydroxybutyrate in the formulation is about 50%. The molar percentage of gamma-hydroxybutyrate in the modified release portion relative to the total of gamma-hydroxybutyrate in the formulation ranges from 90% to 35%, from 85 to 40%, from 80 to 45%, from 75 to 45%, from 70 to 45%, from 65 to 45%, from 60 to 45%, from 60 to 40%, or from 55 to 45%. In an embodiment, the molar percentage of gamma-hydroxybutyrate in the modified release portion relative to the total of gamma-hydroxybutyrate in the formulation ranges from 60% to 40%. In one embodiment, the molar ratio of the gamma-hydroxybutyrate in the modified release portion relative to the total of gamma-hydroxybutyrate in the formulation is about 50%. The weight percentage of the IR microparticles relative to the total weight of IR microparticles and MR microparticles ranges from 7.2% to 58.2%, from 11.0% to 52.9%, from 14.9% to 47.8%, from 18.9% to 47.8%, from 23.1% to 47.8%, from 27.4% to 47.8%, from 31.8% to 47.8%, from 31.8% to 52.9%, or from 36.4% to 47.8%. In other embodiments, the weight percentage of the IR microparticles relative to the total weight of IR microparticles and MR microparticles ranges from 5.9% to 63.2%, from 9.1% to 58.1%, from 12.4% to 53.1%, from 19.9% to 53.1%, from 19.6% to 53.1%, from 23.4% to 53.1%, from 27.4% to 53.1%, or from 27.4% to 58.1%. In one embodiment, the weight percentage of the IR microparticles relative to the total weight of IR microparticles and MR microparticles ranges from 31.7% to 53.1%.

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to 450 microns and 50% of its sodium oxybate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to 170 microns and 50% of its sodium oxybate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone K30 and 15% of microcrystalline cellulose spheres with a volume mean diameter of about 95 microns to about 450 microns and 50% of its sodium oxybate content in modified release particles consisting of 11.3% w/w of microcrystalline cellulose spheres with a volume mean diameter of about 95 microns to about 450 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

In an embodiment, the finished formulation comprises 50% of its sodium oxybate content in immediate-release particles consisting of 80.75% w/w of sodium oxybate, 4.25% w/w of Povidone™ K30 and 15% of microcrystalline cellulose spheres with a volume mean diameter of about 95 microns to about 170 microns and 50% of its sodium oxybate content in modified release particles consisting of 11.3% w/w of microcrystalline cellulose spheres with a volume mean diameter of about 95 microns to about 170 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 16.7% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns, 16.7% of its gamma-hydroxybutyrate content in immediate-release particles consisting of 80.75% w/w of magnesium salt of gamma-hydroxybutyric acid, 4.25% w/w of Povidone K30 and 15% of microcrystalline cellulose spheres with a volume mean diameter of about 95 microns to about 450 microns, 16.7% of its gamma-hydroxybutyrate content in immediate-release particles consisting of 80.75% w/w of calcium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 16.7% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns, 16.7% of its gamma-hydroxybutyrate content in immediate-release particles consisting of 80.75% w/w of magnesium salt of gamma-hydroxybutyric acid, 4.25% w/w of Povidone K30 and 15% of microcrystalline cellulose spheres with a volume mean diameter of about 95 microns to about 170 microns, 16.7% of its gamma-hydroxybutyrate content in immediate-release particles consisting of 80.75% w/w of calcium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 450 microns and 50% of its gamma-hydroxybutyrate content in modified release particles consisting of 10.5% w/w of microcrystalline cellulose spheres with a volume mean diameter of about 95 microns to about 450 microns, layered with 56.5% w/w of calcium salt of gamma-hydroxybutyric acid mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

In an embodiment, the finished formulation comprises 50% of its gamma-hydroxybutyrate content in immediate-release particles consisting of 80.75% w/w of potassium salt of gamma-hydroxybutyric acid, 4.25% w/w of Povidone K30 and 15% of microcrystalline cellulose spheres with a volume mean diameter of about 95 microns to about 170 microns and 50% of its gamma-hydroxybutyrate content in modified release particles consisting of 10.5% w/w of microcrystalline cellulose spheres with a volume mean diameter of about 95 microns to about 170 microns, layered with 56.5% w/w of calcium salt of gamma-hydroxybutyric acid mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent).

### Other Characteristics of Immediate Release Portion

The immediate release portion of the formulation can take any form capable of achieving an immediate release of the gamma-hydroxybutyrate when ingested. For example, when the formulation is a particulate formulation, the formulation can include unmodified “raw” gamma-hydroxybutyrate, rapidly dissolving gamma-hydroxybutyrate granules, particles or microparticles comprised of a core covered by a gamma-hydroxybutyrate loaded layer containing a binder such as povidone.

The IR granules or particles of gamma-hydroxybutyrate may be made using any manufacturing process suitable to produce the required particles, including:
agglomeration of the gamma-hydroxybutyrate sprayed in the molten state, such as the Glatt ProCell technique,
extrusion and spheronization of the gamma-hydroxybutyrate, optionally with one or more physiologically acceptable excipients,
wet granulation of the gamma-hydroxybutyrate, optionally with one or more physiologically acceptable excipients,
compacting of the gamma-hydroxybutyrate, optionally with one or more physiologically acceptable excipients,
granulation and spheronization of the gamma-hydroxybutyrate, optionally with one or more physiologically acceptable excipients, the spheronization being carried out for example in a fluidized bed apparatus equipped with a rotor, in particular using the Glatt CPS™ technique,
spraying of the gamma-hydroxybutyrate, optionally with one or more physiologically acceptable excipients, for example in a fluidized bed type apparatus equipped with zig-zag filter, in particular using the Glatt MicroPx™ technique, or
spraying, for example in a fluidized bed apparatus optionally equipped with a partition tube or Wurster tube, the gamma-hydroxybutyrate, optionally with one or more physiologically acceptable excipients, in dispersion or in solution in an aqueous or organic solvent on a core.

The immediate release portion of the formulation is in the form of microparticles comprising the immediate release gamma-hydroxybutyrate and optional pharmaceutically acceptable excipients. In an embodiment, the immediate release microparticles of gamma-hydroxybutyrate have a volume mean diameter D(4,3) of from 10 to 1000 microns. In other embodiments, the immediate release microparticles of gamma-hydroxybutyrate have a volume mean diameter D(4,3) of from 95 to 600 microns. In additional embodiments, the immediate release microparticles of gamma-hydroxybutyrate have a volume mean diameter D(4,3) of from 150 to 400 microns. In one embodiment, their volume mean diameter is about 270 microns.

The immediate release particles of gamma-hydroxybutyrate may include a core and a layer deposited on the core that contains the gamma-hydroxybutyrate. The core may be any particle chosen from the group consisting of:
crystals or spheres of lactose, sucrose (such as Compressuc™ PS from Tereos), microcrystalline cellulose (such as Avice™ from FMC Biopolymer, Cellet™ from Pharmatrans or Celphere™ from Asahi Kasei), sodium chloride, calcium carbonate (such as Omyapure™ 35 from Omya), sodium hydrogen carbonate, dicalcium phosphate (such as Dicafos™ AC 92-12 from Budenheim) or tricalcium phosphate (such as Tricafos™ SC93-15 from Budenheim);
composite spheres or granules, for example sugar spheres comprising sucrose and starch (such as Suglets™ from NP Pharm), spheres of calcium carbonate and starch (such as Destab™ 90 S Ultra 250 from Particle Dynamics) or spheres of calcium carbonate and maltodextrin (such as Hubercal™ CCG4100 from Huber).

The core can also comprise other particles of pharmaceutically acceptable excipients such as particles of hydroxypropyl cellulose (such as Klucel™ from Aqualon Hercules), guar gum particles (such as Grinsted™ Guar from Danisco), xanthan particles (such as Xantural™ 180 from CP Kelco).

According to a particular embodiment of the invention, the cores are sugar spheres or microcrystalline cellulose spheres, such as Cellets™ 90, Gellets™ 100 or Cellets™ 127 marketed by Pharmatrans, or also Celphere™ OP 203, Gelphere™ CP305, Celphere™ SCP 100. In one embodiment, the core is a microcrystalline cellulose sphere. For example, the core may be a Cellets™ 127 from Pharmatrans.

In various embodiments, the core has a mean volume diameter of about 95 to about 450 microns, about 95 to about 170 microns, or about 140 microns.

The layer deposited onto the core comprises the immediate release gamma-hydroxybutyrate. In an embodiment, the layer also comprises a binder, which may be chosen from the group consisting of:
low molecular weight hydroxypropyl cellulose (such as Klucel™ EF from Aqualon-Hercules), low molecular weight hydroxypropyl methylcellulose (or hypromellose) (such as Methocel™ E3 or E5 from Dow), or low molecular weight methylcellulose (such as Methocel™ A15 from Dow);
low molecular weight polyvinyl pyrrolidone (or povidone) (such as Plasdone™ K29/32 from ISP or Kollidon™ 30 from BASF), vinyl pyrrolidone and vinyl acetate copolymer (or copovidone) (such as Plasdone™ S630 from ISP or Kollidon™ VA 64 from BASF);
dextrose, pregelatinized starch, maltodextrin; and mixtures thereof.

Low molecular weight hydroxypropyl cellulose corresponds to grades of hydroxypropyl cellulose having a molecular weight of less than 800,000 g/mol, less than or equal to 400,000 g/mol, or less than or equal to 100,000 g/mol. Low molecular weight hydroxypropyl methylcellulose (or hypromellose) corresponds to grades of hydroxypropyl methylcellulose the solution viscosity of which, for a 2% solution in water and at 20° C., is less than or equal to 1,000 mPa·s, less than or equal to 100 mPa·s, or less than or equal to 15 mPa·s. Low molecular weight polyvinyl pyrrolidone (or povidone) corresponds to grades of polyvinyl pyrrolidone having a molecular weight of less than or equal to 1,000,000 g/mol, less than or equal to 800,000 g/mol, or less than or equal to 100,000 g/mol.

In some embodiments, the binding agent is chosen from low molecular weight polyvinylpyrrolidone or povidone (for example, Plasdone™ K29/32 from ISP), low molecular weight hydroxypropyl cellulose (for example, Klucel™ EF from Aqualon-Hercules), low molecular weight hydroxypropyl methylcellulose or hypromellose (for example, Methocel™ E3 or E5 from Dow) and mixtures thereof.

In one embodiment, the binder is povidone K30 or K29/32, especially Plasdone™ K29/32 from ISP. The binder may be present in an amount of 0 to 80%, 0 to 70%, 0 to 60%, 0 to 50%, 0 to 40%, 0 to 30%, 0 to 25%, 0 to 20%, 0 to 15%, 0 to 10%, or from 1 to 9% of binder based on the total weight of the immediate release coating. In an embodiment, the binder is present in an amount of 5% based on the total weight of the immediate release coating. In one embodiment, the amount of binder is 5% of binder over the total mass of gamma-hydroxybutyrate and binder.

The layer deposited on the core can represent at least 10% by weight, and even greater than 20, 25, 30, 35, 40, 45, 50, 55, 60, 65, 70, 75, 80, 85 or 90% by weight of the total weight of the immediate release particle of gamma-hydroxybutyrate. In one embodiment, the layer deposited on the core represents about 85% of the weight of the immediate release particle of gamma-hydroxybutyrate.

According to an embodiment, the immediate-release particles comprise 80.75% w/w of gamma-hydroxybutyrate, 4.25% w/w of Povidone K30 and 15% of microcrystalline cellulose spheres.

According to another embodiment, the immediate-release particles comprise 80.75% w/w of gamma-hydroxybutyrate, 4.25% w/w of Povidone K30 and 15% of microcrystalline cellulose spheres with a volume mean diameter of about 95 microns to about 450 microns.

According to yet another embodiment, the immediate-release particles comprise 80.75% w/w of gamma-hydroxybutyrate, 4.25% w/w of Povidone K30 and 15% of microcrystalline cellulose spheres with a volume mean diameter of about 95 microns to about 170 microns.

According to an embodiment, the immediate-release particles comprise 80.75% w/w of sodium oxybate, 4.25% w/w of Povidone K30 and 15% of microcrystalline cellulose spheres.

According to another embodiment, the immediate-release particles comprise 80.75% w/w of potassium salt of gamma-hydroxybutyric acid, 4.25% w/w of Povidone K30 and 15% of microcrystalline cellulose spheres.

According to another embodiment, the immediate-release particles comprise 80.75% w/w of calcium salt of gamma-hydroxybutyric acid, 4.25% w/w of Povidone K30 and 15% of microcrystalline cellulose spheres.

According to another embodiment, the immediate-release particles comprise 80.75% w/w of magnesium salt of gamma-hydroxybutyric acid, 4.25% w/w of Povidone K30 and 15% of microcrystalline cellulose spheres.

According to another embodiment, the immediate-release particles are manufactured by dissolving the gamma-hydroxybutyrate and the Povidone K30 in a mixture of water/ethanol 40/60 w/w and spraying the resulting solution onto the surface of the microcrystalline cellulose spheres.

### Other Characteristics of Modified Release Portion

The modified release portion may be any formulation that provides the desired in vitro dissolution profile of gamma-hydroxybutyrate. The modified release portion may include modified release particles, obtained by coating immediate release particles of gamma-hydroxybutyrate with a coating (or coating film) that inhibits the immediate release of the gamma-hydroxybutyrate. In one sub-embodiment the modified release portion comprises particles comprising: (a) an inert core; (b) a coating; and (c) a layer comprising the gamma hydroxybutyrate interposed between the core and the coating.

In an embodiment, the modified release portion comprises a time-dependent release mechanism and a pH-dependent release mechanism.

In an embodiment, the coating film comprises at least one polymer carrying free carboxylic groups, and at least one hydrophobic compound characterized by a melting point equal or greater than 40° C.

The polymer carrying free carboxylic groups may be selected from: (meth)acrylic acid/alkyl (meth)acrylate copolymers or methacrylic acid and methylmethacrylate copolymers or methacrylic acid and ethyl acrylate copolymers or methacrylic acid copolymers type A, B or C, cellulose derivatives carrying free carboxylic groups, cellulose acetate phthalate, cellulose acetate succinate, hydroxypropyl methyl cellulose phthalate, carboxymethylethyl cellulose, cellulose acetate trimellitate, hydroxypropyl methyl cellulose acetate succinate, polyvinyl acetate phthalate, zein, shellac, alginate and mixtures thereof.

In an embodiment, the methacrylic acid copolymers are chosen from the group consisting of poly (methacrylic acid, methyl methacrylate) 1:1 or Eudragit™ L100 or equivalent, poly (methacrylic acid, ethyl acrylate) 1:1 or Eudragit™ L100-55 or equivalent and poly (methacrylic acid, methyl methacrylate) 1:2 or Eudragit™ S100 or equivalent.

In another embodiment the coating comprises a polymer carrying free carboxylic groups wherein the free carboxylic groups are substantially ionized at pH 7.5.

The hydrophobic compound with a melting point equal or greater than 40° C. may be selected from the group consisting of hydrogenated vegetable oils, vegetable waxes, wax yellow, wax white, wax microcrystalline, lanolin, anhydrous milk fat, hard fat suppository base, lauroyl macrogol glycerides, polyglyceryl diisostearate, diesters or triesters of glycerol with a fatty acid, and mixtures thereof.

In various embodiments, the hydrophobic compound with a melting point equal or greater than 40° C. is chosen from the group of following products: hydrogenated cottonseed oil, hydrogenated soybean oil, hydrogenated palm oil, glyceryl behenate, hydrogenated castor oil, candelilla wax, tristearin, tripalmitin, trimyristin, yellow wax, hard fat or fat that is useful as suppository bases, anhydrous dairy fats, lanolin, glyceryl palmitostearate, glyceryl stearate, lauryl macrogol glycerides, polyglyceryl diisostearate, diethylene glycol monostearate, ethylene glycol monostearate, omega 3 fatty acids, and mixtures thereof. For example, the hydrophobic compound may include hydrogenated cottonseed oil, hydrogenated soybean oil, hydrogenated palm oil, glyceryl behenate, hydrogenated castor oil, candelilla wax, tristearin, tripalmitin, trimyristin, beeswax, hydrogenated poly-1 decene, carnauba wax, and mixtures thereof.

In practice, and without this being limiting, the hydrophobic compound with a melting point equal or greater than 40° C. may be chosen from the group of products sold under the following trademarks: Dynasan™, Cutina™, Hydrobase™, Dub™, Castorwax™, Croduret™, Compritol™, Sterotex™, Lubritab™, Apifil™, Akofine™, Softisan™, Hydrocote™, Livopol™, Super Hartolan™, MGLA™, Corona™, Protalan™, Akosoft™, Akosol™, Cremao™, Massupol™, Novata™, Suppocire™, Wecobee™, Witepsol™, Lanolin™, Incromega™, Estaram™, SuppoweiSS™, Gelucire™, Precirol™, Emulcire™, Plurol Diisostéarique™, Geleol™, Hydrine™, Monthyle™, Kahlwax™ and mixtures thereof. In an embodiment, the hydrophobic compound with a melting point equal or greater than 40° C. may be chosen from the group of products sold under the following trademarks: Dynasan™ P60, Dynasan™114 Dynasan™116, Dynasan™118, Cutina™ HR, Hydrobase™ 66-68, Dub™ HPH, Compritol™ 888, Sterotex™ NF, Sterotex™ K, Lubritab™, and mixtures thereof.

A particularly suitable coating is composed of a mixture of hydrogenated vegetable oil and a methacrylic acid copolymer. The exact structure and amount of each component, and the amount of coating applied to the particle, controls the release rate and release triggers. Eudragit® methacrylic acid copolymers, namely the methacrylic acid-methyl methacrylate copolymers and the methacrylic acid-ethyl acrylate copolymers, have a pH-dependent solubility: typically, the pH triggering the release of the active ingredient from the microparticles is set by the choice and mixture of appropriate Eudragit® polymers. In the case of gamma hydroxybutyrate modified release microparticles, the theoretical pH triggering the release is from 5.5 to 6.97 or from 5.5 to 6.9. By “pH trigger” is meant the minimum pH above which dissolution of the polymer occurs.

In a particular embodiment, the coating comprises a hydrophobic compound with a melting point equal or greater than 40° C. and a polymer carrying free carboxylic groups are present in a weight ratio from 0.4 to 4, from 0.5 to 4, from 0.6 to 2.5, from 0.67 to 2.5, from 0.6 to 2.33, or from 0.67 to 2.33. In one embodiment, the weight ratio is about 1.5.

A particularly suitable coating is composed of a mixture of hydrogenated vegetable oil and a methacrylic acid copolymer with a theoretical pH triggering the release from 6.5 up to 6.97 in a weight ratio from 0.4 to 4, from 0.5 to 4, from 0.6 to 2.5, from 0.67 to 2.5, from 0.6 to 2.33, or from 0.67 to 2.33. In one embodiment, the weight ratio may be about 1.5.

The modified release particles of gamma-hydroxybutyrate have a volume mean diameter of from 100 to 1200 microns, from 100 to 500 microns, or from 200 to 800 microns. In one embodiment, the modified release particles of gamma-hydroxybutyrate have a volume mean diameter of about 320 microns.

The coating can represent 10 to 50%, 15 to 45%, 20 to 40%, or 25 to 35% by weight of the total weight of the coated modified release particles. In one embodiment, the coating represents 25-30% by weight of the total weight of the modified release particles of gamma-hydroxybutyrate.

In an embodiment, the coating layer of the modified release particles of gamma-hydroxybutyrate is obtained by spraying, in particular in a fluidized bed apparatus, a solution, suspension or dispersion comprising the coating composition as defined previously onto the immediate release particles of gamma-hydroxybutyrate, in particular the immediate release particles of gamma-hydroxybutyrate as previously described. In one embodiment, the coating is formed by spraying in a fluidized bed equipped with a Wurster or partition tube and according to an upward spray orientation or bottom spray a solution of the coating excipients in hot isopropyl alcohol.

According to an embodiment, the modified release particles of gamma-hydroxybutyrate consist of 10.5% w/w of microcrystalline cellulose spheres with a volume mean diameter of about 95 microns to about 450 microns, layered with 56.5% w/w of gamma-hydroxybutyrate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of gamma-hydroxybutyrate.

According to an embodiment, the modified release particles of gamma-hydroxybutyrate consist of 10.5% w/w of microcrystalline cellulose spheres with a volume mean diameter of about 95 microns to about 170 microns, layered with 56.5% w/w of gamma-hydroxybutyrate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of gamma-hydroxybutyrate.

According to an embodiment, the modified release particles of gamma-hydroxybutyrate consist of 10.5% w/w of microcrystalline cellulose spheres with a volume mean diameter of about 95 microns to about 450 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of sodium oxybate.

According to an embodiment, the modified release particles of gamma-hydroxybutyrate consist of 10.5% w/w of microcrystalline cellulose spheres with a volume mean diameter of about 95 microns to about 170 microns, layered with 56.5% w/w of sodium oxybate mixed with 3% w/w of Povidone™ K30 and finally coated with a coating composition consisting of 18% w/w of hydrogenated vegetable oil (Lubritab™ or equivalent), 4% of methacrylic acid copolymer type C (Eudragit™ L100-55 or equivalent) and 8% of methacrylic acid copolymer type B (Eudragit™ S100 or equivalent), all percentages expressed based on the total weight of the final modified release particles of sodium oxybate.

According to another embodiment, the modified release particles of gamma-hydroxybutyrate consist of 11.3% w/w of microcrystalline cellulose spheres with a volume mean diameter of about 95 microns to about 450 microns, layered with 60.5% w/w of gamma-hydroxybutyrate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 170 microns, layered with 60.5% w/w of gamma-hydroxybutyrate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 450 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

According to another embodiment, the modified release particles of gamma-hydroxybutyrate consist of 11.3% w/w of microcrystalline cellulose spheres with a volume mean diameter of about 95 microns to about 170 microns, layered with 60.5% w/w of sodium oxybate mixed with 3.2% w/w of Povidone™ K30 and finally coated with a coating composition consisting of 15% w/w of hydrogenated vegetable oil (Lubritab™ or equivalent), 0.75% of methacrylic acid copolymer type C (Eudragit™ L100-55 or equivalent) and 9.25% of methacrylic acid copolymer type B (Eudragit™ S100 or equivalent).

### Soft Food Vehicles

In addition to water, the composition may be mixed with alternative soft foods/vehicles known in the art to form a suspension or mixture of the composition suitable for administration to a human patient in need thereof. As used herein, when a soft food vehicle is used, a combination with a pharmaceutical composition may form a suspension or a mixture, which may be used interchangeably. A method of treating narcolepsy, cataplexy, excessive daytime sleepiness, or a sleep related disorder in a human patient may include forming a mixture comprising a pharmaceutical composition and a soft food vehicle, and orally administering the mixture to the human patient. In some aspects, the orally administering occurs only once nightly.

In some embodiments, the pharmaceutical composition may include an amount of gamma-hydroxybutyrate equivalent to 3.0 g to 12.0 g of sodium oxybate. For example, the gamma-hydroxybutyrate may be in an amount equivalent to 3.0 g, 4.5 g, 6.0 g, 7.5 g, 9.0 g, 10.5 g, or 12.0 g of sodium oxybate. As disclosed herein above, in some examples, the gamma-hydroxybutyrate composition may include an immediate release portion and a modified release portion. The pharmaceutical composition may be a dry particulate formulation or a powdered formulation. In various embodiments, the pharmaceutical composition includes carrageenan, hydrogenated vegetable oil, hydroxyethyl cellulose, magnesium stearate, malic acid, methacrylic acid copolymer, microcrystalline cellulose, povidone, and xanthan gum.

In some embodiments, the mixture has a pH of less than 7.0, less than 6.9, less than 6.8, less than 6.7, less than 6.6, less than 6.5, less than 6.4, less than 6.3, less than 6.2, less than 6.2, less than 6.1, less than 6.0, less than 5.9, less than 5.8, less than 5.7, less than 5.6, less than 5.5, less than 5.4, less than 5.3, less than 5.2, less than 5.1, less than 5.0, less than 4.9, less than 4.8, less than 4.7, less than 4.6, or less than 4.5 when the composition is mixed with the soft food vehicle. In other embodiments, the mixture has a pH of 4.5 to 5.7, 5.0 to 6.0, 5.0 to 5.7, 5.2 to 5.7, 5.2 to 5.9, 5.2 to 5.7, 5.3 to 5.5, 5.5 to 5.9, 5.5 to 5.7, 5.5 to 6.0, 5.5 to 6.5, or 5.5 to 6.9 when the composition is mixed with the soft food vehicle. In additional embodiments, the mixture may have a pH of 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, or 4.5 when the composition is mixed with the soft food vehicle. The pH of the mixture is important to maintaining the pharmaceutical composition and not dissolving the coating or otherwise affecting the effectiveness of the pharmaceutical composition. For example, a pH of above 7.0, above 6.5, or above 6.0 may cause dissolution of the coating on the modified release portion of the pharmaceutical composition. In other examples, a pH below 5.0. below 4.5, or below 4.0 may be detrimental to the composition. In various embodiments, the pH may be maintained for at least 5 minutes, up to 10 minutes, up to 15 minutes, up to 20 minutes, up to 25 minutes, up to 30 minutes, or greater than 30 minutes after forming the suspension. In other embodiments, the pH may be maintained for at least 5 minutes, at least 10 minutes, at least 15 minutes, at least 20 minutes, at least 25 minutes, or at least 30 minutes after forming the suspension. In one example, the pH of the suspension may be 5.5 to 5.7 for up to 30 minutes after forming the mixture. In some embodiments, the administering occurs within 30 minutes of forming the suspension.

A soft food vehicle or soft food for reconstitution means any food when mixed with the composition has a pH of 4.0-7.0, preferably 5.0 to 6.0. Other soft foods that are not vehicles or for reconstitution (e.g. outside the pH range) may break down the coating of the composition, deteriorate the API, or otherwise negatively impact its bioavailability. Through testing, it was found that mixtures of the composition with soft food vehicles with a final pH of about 5.0 to 6.0 provide bioequivalent release to a suspension of the composition in water. Bioequivalent with soft food vehicles means bioequivalent to the same composition when suspended in water instead of the soft food. In some aspects, a soft food vehicle is safe and effective to be administered when mixed with the composition. In other aspects, the soft food vehicle does not dissolve the composition. The soft food vehicle may be soft foods such as pudding, yogurt, applesauce, or other soft foods capable of being mixed with the composition to form a suspension. The soft food vehicle may include a flavor additive, natural flavoring, or artificial flavoring. The total amount of flavor additive, natural flavoring, and/or artificial flavoring in the soft food vehicle may be less than 10% (w/w), less than 5% (w/w), or less than 2% (w/w) of the mixture. In one example, the soft food vehicle may be lemon pudding. In another example, the soft food vehicle may be vanilla yogurt.

In an embodiment, the pharmaceutical composition may be mixed with an amount of the soft food vehicle that is equivalent to the amount of water that the pharmaceutical composition would be mixed with to form a suspension. Although the exact volume of soft food vehicle mixed with the composition may vary, in some examples, the pharmaceutical composition may be mixed with about 25 mL, about 50 mL, about 75 mL, about 100 mL, or the amount in a pre-packaged single serving of the soft food. In some aspects, the container with the mixture may be rinsed with water and consumed to confirm that all of the composition has been administered. For example, after the mixture has been administered, the container may be rinsed with about 10 mL, about 25 mL, about 50 mL, or about 75 mL. In some cases, the rinsing step may be repeated. In some examples, the soft food vehicle may be measured and mixed with the composition in the dosing cup. In other examples, the soft food vehicle may be mixed with the composition in the packaging of the soft food vehicle or another container.

In another embodiment, a method of treating a disorder treatable with gamma-hydroxybutyrate in a human in need thereof may include opening a sachet containing a single daily dose of a pharmaceutical composition, mixing the pharmaceutical composition with a soft food vehicle, and orally administering the mixture. The pharmaceutical composition includes an amount of gamma-hydroxybutyrate equivalent to from 3.0 to 12.0 g of sodium oxybate. In some embodiments, the pharmaceutical composition is in immediate-release and modified-release portions. The opening, mixing, and orally administering occur only once daily.

In some embodiments, a pharmaceutical mixture may include an immediate release portion comprising gamma-hydroxybutyrate, a modified release portion comprising gamma-hydroxybutyrate, a suspending or viscosifying agent selected from the group consisting of xanthan gum, carrageenan gum, gellan gum, guar gum, sodium alginate, calcium alginate, agar, sodium carboxymethyl cellulose, microcrystalline cellulose, hydroxyethyl cellulose, hydroxypropylmethyl cellulose, and mixtures thereof, an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid, and a soft food vehicle. The modified release portion may include particles of gamma-hydroxybutyrate coated with a coating including a polymer carrying free carboxylic groups, and a hydrophobic compound having a melting point equal or greater than 40° C. The ratio of gamma-hydroxybutyrate in the immediate release portion and the modified release portion may be from 10/90 to 65/35.

In various embodiments, the mixture may have a pH of 5.2 to 5.8 after 5 minutes, 10 minutes, 15 minutes, 20 minutes, 25 minutes, or 30 minutes when suspended in a soft food vehicle. In other embodiments, the mixture may have a pH of 5.3 to 5.5 after 5 minutes, 10 minutes, 15 minutes, 20 minutes, 25 minutes, or 30 minutes when suspended in a soft food vehicle.

There may be no impact on the dissolution profile of the pharmaceutical composition or not detrimental effects on the pharmaceutical composition when mixed with a soft food vehicle when the mixture has a pH of 4.0 to 7.0 or 5.0 to 6.0. Having no impact on the dissolution profile means there is little to no dissolution of the modified release portion or the modified release portion does not dissolve within 5-30 min when mixed in the soft food vehicle. Alternatively, having no impact on the dissolution profile means the dissolution profile of the pharmaceutical composition when mixed in a soft food vehicle is equivalent or bioequivalent to the same pharmaceutical composition suspended in water (e.g. tap water or Milli-Q purified water). In some embodiments, the pharmaceutical composition in a soft food vehicle provides any one of the dissolution profiles as described herein. For example, the pharmaceutical composition in a soft food vehicle may have an equivalent or bioequivalent dissolution profile to the pharmaceutical composition in water. The mixture with the soft food vehicle may be bioequivalent to when the composition is suspended in water.

In some examples, the mixture provides an improved mouthfeel relative to when the composition is suspended in water. In other examples, the mixture provides an improved taste as compared to when the composition is suspended in water.

### Packaging

The composition of gamma-hydroxybutyrate may be supplied in sachets or stick-packs comprising a particulate formulation. The sachets may be available in several different doses, comprising gamma-hydroxybutyrate in amounts equivalents to 0.5 g, 1.0 g, 1.5 g, 3.0 g, 4.5 g, 6.0 g, 7.5 g, 9.0 g, 10.5 g and/or 12 g of sodium oxybate. Depending on the dose required, one or more of these sachets may be opened, and its contents mixed with tap water to provide the nightly dose of gamma-hydroxybutyrate.

### Methods of Treatment

Further provided herein is a method of treating a disorder treatable with gamma-hydroxybutyrate in a human subject in need thereof comprising orally administering, less than two hours after eating, a single bedtime daily dose to said human amounts of gamma-hydroxybutyrate equivalent to from 3.0 to 12.0 g of sodium oxybate in the composition. Further provided herein are methods of treating narcolepsy, types 1 and/or 2, by orally administering at bedtime a therapeutically effective amount of a gamma-hydroxybutyrate formulation characterized by the novel gamma-hydroxybutyrate pharmacokinetic properties of the composition when administered less than two hours after eating. In an embodiment, the composition of the present invention is effective to treat narcolepsy Type 1 or Type 2, wherein the treatment of narcolepsy is defined as reducing excessive daytime sleepiness or reducing the frequency of cataplectic attacks. The therapeutically effective amount may include equivalents from 3.0 to 12.0 g of sodium oxybate. In various embodiments, the therapeutically effective amount is 4.5, 6.0, 7.5 or 9.0 g of sodium oxybate. In one embodiment, the therapeutically effective amount is 6 g or 9 g of sodium oxybate. In various embodiments, the formulation includes sodium oxybate present in a unit dose of at least 4.5 g, at least 6.0 g, at least 7.5 g, or at least 9.0 g. The effectiveness of the treatment may be measured by one or any combination of the following criteria:
Increase the mean sleep latency, as determined on the Maintenance of Wakefulness Test (MWT)
Improve the Clinical Global Impression (CGI) rating of sleepiness
Decrease the number of cataplexy attacks (NCA) determined from the cataplexy frequency item in the Sleep and Symptoms Daily Diary
Decrease the disturbed nocturnal sleep (DNS), the disturbed nocturnal events or the adverse respiratory events as determined by polysomnographic (PSG) measures of sleep fragmentation
Decrease the excessive daytime sleepiness (EDS) as measured by patient report via the Epworth Sleepiness Scale (ESS)
Decrease the daytime sleepiness as measured by the Maintenance of Wakefulness Test based on EEG measures of wakefulness
Decrease PSG transitions from N/2 to N/3 and REM sleep to wake and N1 sleep (as determined by C Iber, S Ancoli-Israel, A Chesson, SF Quan. The AASM Manual for the Scoring of Sleep and Associated Events. Westchester, IL: American Academy of Sleep Medicine; 2007).
Decrease the number of arousals or wakenings, obtained from a PSG as defined by the American Academy of Sleep Medicine
Improve the sleep quality, obtained from one or more of (i) the Sleep and Symptom Daily Diary, (ii) Visual Analog Scale (VAS) for sleep quality and sleep diary, and (iii) VAS for the refreshing nature of sleep
Decrease the Hypnagogic Hallucinations (HH) or sleep paralysis (SP) symptoms in NT1 narcolepsy patients, as measured by the Sleep and Symptom Daily Diary

In an embodiment, the treatment using the composition administered less than two hours after eating is superior, as measured by any one or combination of the foregoing criteria, to an equal dose administered twice nightly of an immediate release liquid solution of sodium oxybate, with the second dose administered 4 hours after the first dose.

The invention further provides a method of treatment of narcolepsy Type 1 or Type 2 wherein, compared to a dosing regimen consisting of administering half the dose at t0 and another half of the dose at t4 h of an immediate release liquid solution of sodium oxybate, a single bedtime daily dose administration of a therapeutically effective amount of the formulation of the invention has been shown to produce less confusion, less depressive syndrome, less incontinence, less nausea or less sleepwalking.

### EXAMPLES

### Example 1. Formulations

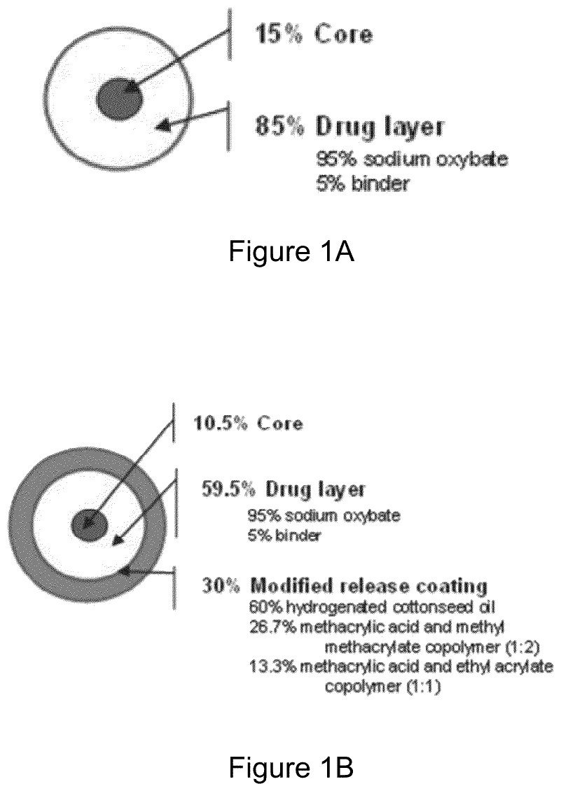

Tables 1a-1d provide the qualitative and quantitative compositions of sodium oxybate IR microparticles, MR microparticles, and mixtures of IR and MR microparticles. The physical structure of the microparticles showing the qualitative and quantitative composition of the IR and MR microparticles is depicted in FIG. 1.

Briefly, sodium oxybate immediate release (IR) microparticles were prepared as follows: 1615.0 g of sodium oxybate and 85.0 g of polyvinylpyrrolidone (Povidone K30-Plasdone™ K29/32 from ISP) were solubilized in 1894.3 g of absolute ethyl alcohol and 1262.9 g of water. The solution was entirely sprayed onto 300 g of microcrystalline cellulose spheres (Cellets™ 127) in a fluid bed spray coater apparatus. IR Microparticles with volume mean diameter of about 270 microns were obtained.

Sodium oxybate modified release (MR) microparticles were prepared as follows: 22.8 g of methacrylic acid copolymer Type C (Eudragit™ L100-55), 45.8 g of methacrylic acid copolymer Type B (Eudragit™ S100), 102.9 g of hydrogenated cottonseed oil (Lubritab™), were dissolved in 1542.9 g of isopropanol at 78° C. The solution was sprayed entirely onto 400.0 g of the sodium oxybate IR microparticles described above in a fluid bed spray coater apparatus with an inlet temperature of 48° C., spraying rate around 11 g per min and atomization pressure of 1.3 bar. MR microparticles were dried for two hours with inlet temperature set to 56° C. MR microparticles with mean volume diameter of about 320 microns were obtained.

The finished composition, which contains a 50:50 mixture of MR and IR microparticles calculated on their sodium oxybate content, was prepared as follows: 353.36 g of the above IR microparticles, 504.80 g of the above MR microparticles, 14.27 g of malic acid (D/L malic acid), 6.34 g of xanthan gum (Xantural™ 75 from Kelco), 9.51 g of carrageenan gum (Viscarin™ PH209 from FMC Biopolymer), 9.51 g of hydroxyethylcellulose (Natrosol™ 250M from Ashland) and 4.51 g of magnesium stearate were mixed. Individual samples of 7.11 g (corresponding to a 4.5 g dose of sodium oxybate with half of the dose as immediate-release fraction and half of the dose as modified release fraction) were weighed.

TABLE 1a
Composition of IR Microparticles

| | | Quantity per |
|---|---|---|
| Component | Function | 2.25 g dose (g) |
| Sodium oxybate | Drug substance | 2.25 |
| Microcrystalline | Core | 0.418 |
| cellulose spheres | | |
| Povidone K30 | Binder and excipient | 0.118 |
| | in diffusion coating | |
| Ethyl alcohol | Solvent | Eliminated during |
| | | processing |
| Purified water | Solvent | Eliminated during |
| | | processing |
| Total | | 2.786 |

TABLE 1b
Composition of MR Microparticles

| | | Quantity per |
|---|---|---|
| Component | Function | 4.5 g dose (g) |
| IR Microparticles | Core of MR | 2.786 |
| | microparticles | |
| Hydrogenated Vegetable Oil | Coating excipient | 0.716 |
| Methacrylic acid | Coating excipient | 0.159 |
| Copolymer Type C | | |
| Methacrylic acid Copolymer Type B | Coating excipient | 0.318 |
| Isopropyl alcohol | Solvent | Eliminated during |
| | | processing |
| Total | | 3.981 |

TABLE 1c
Qualitative Finished Composition

| | | Quantity per |
|---|---|---|
| Component | Function | 4.5 g dose (g) |
| MR microparticles | Modified release fraction | 3.981 |
| | of sodium oxybate | |
| IR microparticles | Immediate release | 2.786 |
| | fraction of sodium | |
| | oxybate | |
| Malic acid | Acidifying agent | 0.113 |
| Xanthan gum | Suspending agent | 0.050 |
| Hydroxyethylcellulose | Suspending agent | 0.075 |
| Carrageenan gum | Suspending agent | 0.075 |
| Magnesium stearate | Lubricant | 0.036 |
| Total | | 7.116 |

TABLE 1d
Quantitative finished composition

| | | Quantity per |
|---|---|---|
| Component | Function | 4.5 g dose (g) |
| Sodium oxybate | Drug substance | 4.5 |
| Microcrystalline | Core | 0.836 |
| cellulose spheres | | |
| Povidone K30 | Binder | 0.237 |
| Hydrogenated Vegetable Oil | Coating excipient | 0.716 |
| Methacrylic acid | Coating excipient | 0.159 |
| Copolymer Type C | | |
| Methacrylic acid | Coating excipient | 0.318 |
| Copolymer Type B | | |
| Malic acid | Acidifying agent | 0.113 |
| Xanthan gum | Suspending agent | 0.050 |
| Hydroxyethylcellulose | Suspending agent | 0.075 |
| Carrageenan gum | Suspending agent | 0.075 |
| Magnesium stearate | Lubricant | 0.036 |
| Total | | 7.116 |

### Example 1bis. Alternative Formulation

An alternative formulation to the formulation described in Example 1 is described in Example 1bis.

Sodium oxybate immediate release (IR) microparticles were prepared by coating the IR microparticles described in Example 1 with a top coat layer. Microparticles were prepared as follows: 170.0 of hydroxypropyl cellulose (Klucel™ EF Pharm from Hercules) were solubilized in 4080.0 g of acetone. The solution was entirely sprayed onto 1530.0 g of the IR microparticles of Example 1 in a fluid bed spray coater apparatus. IR Microparticles with volume mean diameter of about 298 microns were obtained (see Table 1bis-a).

Sodium oxybate modified release (MR) microparticles were prepared as described in example 1 (see Table 1 b).

The finished composition, which contains a 50:50 mixture of MR and IR microparticles based on their sodium oxybate content, was prepared as follows: 412.22 g of the above IR microparticles, 530.00 g of the above MR microparticles, 29.96 g of malic acid (D/L malic acid), 4.96 g of xanthan gum (Xantural™ 75 from Kelco), 4.96 g of colloidal silicon dioxide (Aerosil™ 200 from Degussa) and 9.92 g of magnesium stearate were mixed. Individual samples of 7.45 g (corresponding to a 4.5 g dose of sodium oxybate with half of the dose in an immediate-release fraction and half of the dose in a modified release fraction) were weighed (see Table 1bis-b and 1bis-c).

TABLE 1bis-a
Composition of IR Microparticles

| | | Quantity per |
|---|---|---|
| Component | Function | 2.25 g dose (g) |
| Sodium oxybate | Drug substance | 2.25 |
| Microcrystalline | Core | 0.418 |
| cellulose spheres | | |
| Povidone K30 | Binder and excipient | 0.118 |
| | in diffusion coating | |
| Hydroxypropyl cellulose | Top coat | 0.310 |
| Ethyl alcohol | Solvent | Eliminated during |
| | | processing |
| Purified water | Solvent | Eliminated during |
| | | processing |
| Acetone | Solvent | Eliminated during |
| | | processing |
| Total | | 3.096 |

TABLE 1bis-b
Qualitative Finished Composition

| | | Quantity per |
|---|---|---|
| Component | Function | 4.5 g dose (g) |
| MR microparticles | Modified release fraction | 3.981 |
| | of sodium oxybate | |
| IR microparticles | Immediate release fraction | 3.096 |
| | of sodium oxybate | |
| Malic acid | Acidifying agent | 0.225 |
| Xanthan gum | Suspending agent | 0.037 |
| Colloidal silicon dioxide | Gliding agent | 0.037 |
| Magnesium stearate | Lubricant | 0.075 |
| Total | | 7.451 |

TABLE 1bis-c
Quantitative finished composition

| | | Quantity per |
|---|---|---|
| Component | Function | 4.5 g dose (g) |
| Sodium oxybate | Drug substance | 4.5 |
| Microcrystalline | Core | 0.836 |
| cellulose spheres | | |
| Povidone K30 | Binder | 0.237 |
| Hydroxypropyl cellulose | Top coat | 0.310 |
| Hydrogenated Vegetable Oil | Coating excipient | 0.716 |
| Methacrylic acid | Coating excipient | 0.159 |
| Copolymer Type C | | |
| Methacrylic acid | Coating excipient | 0.318 |
| Copolymer Type B | | |
| Malic acid | Acidifying agent | 0.225 |
| Xanthan gum | Suspending agent | 0.037 |
| Colloidal silicon dioxide | Gliding agent | 0.037 |
| Magnesium stearate | Lubricant | 0.075 |
| Total | | 7.451 |

Compared to the finished composition described in Example 1, this alternative composition has the following characteristics: same MR microparticles, same IR microparticles but with a top coat, increased amount of malic acid, only one suspending agent (xanthan gum) and presence of a glidant.

### Example 2. In Vivo Pharmacokinetic Study of Finished Composition According to Example 1 (6 q FT218 in Fed and Fasted State)

Pharmacokinetic testing was undertaken in vivo in healthy human volunteers. A test product with the finished composition of Example 1 (FT218) and manufactured at larger scale was administered as a 6 g dose. FT218 was administered to 16 healthy volunteers and a first group of n=14 was administered FT218 in the fed state and a second group of n=13 was administered FT218 in the fasted state. The tested samples were manufactured as described in Table 1c for 4.5 g and quantities were hypothetically adjusted to a 6 g dose. The concentration versus time curve of FT218 in the fed and fasted state are presented in FIGS. 2A and 2B. The derived PK parameters are summarized below in Table 2 and Table 3 provides the % ratio of geometric means between the fed and fasted states. FIG. 2A and Table 3 show a Cₘₐₓ(fed)/Cₘₐₓ(fasted)=66.7% and an AUC(fed)/AUC(fasted)=86% for FT218. Therefore, the AUC falls within the 80-125% bioequivalence range with no effect boundaries, so food should have no impact on total exposure of the composition. It appears that food may have no or low impact on MR microparticle absorption in the latter part of the gastrointestinal tract.

TABLE 2
Mean PK Parameters

| | Tmax | Cmax | AUC₀₋ₗₐₛₜ | AUC_{0-inf} | AUC₀₋₈ₕ | C8 h | | |
|---|---|---|---|---|---|---|---|---|
| | (h) | (μg/mL) ± SD | (μg/mL · h) ± SD | (μg/mL · h) ± SD | (μg/mL · h) ± SD | (μg/mL) ± SD | T_{1/2} (h) ± SD | Λz (/h) ± SD |
| Arm | [min-max] | (CV) | (CV) | (CV) | (CV) | (CV) | (CV) | (CV) |
| FT218 | 1.5 | 64.0 ± 17.5 | 241 ± 88.1 | 242 ± 88.2 | 239 ± 85.0 | 2.09 ± 3.14 | 0.71 ± 0.25 | 1.07 ± 0.305 |
| fed | [0.5-2.5] | (27.3) | (36.5) | (36.5) | (35.5) | (150.5) | (34.5) | (28.5) |
| n = 14 | | | | | | | | |
| FT218 | 0.53 | 90.5 ± 15.8 | 267 ± 85.2 | 267 ± 85.2 | 266 ± 82.9 | 1.43 ± 2.04 | | |
| fasted | [0.33-1] | (17.5) | (31.9) | (32) | (31.2) | (142.7) | | |
| n = 13 | | | | | | | | |

TABLE 3
Food Effect on FT218

| | PE | |
|---|---|---|
| | (fed/fasted) | 90% CI |
| Cₘₐₓ | 66.7 | 58.2-76.5 |
| AUC₀₋ₗₐₛₜ | 86.0 | 79.9-92.6 |
| AUC_{0-inf} | 86.1 | 80.0-92.7 |

### Example 3. Simulated Data to Demonstrate the Food Effect on the IR Portion, MR Portion, and FT218 in the Fed and Fasted State

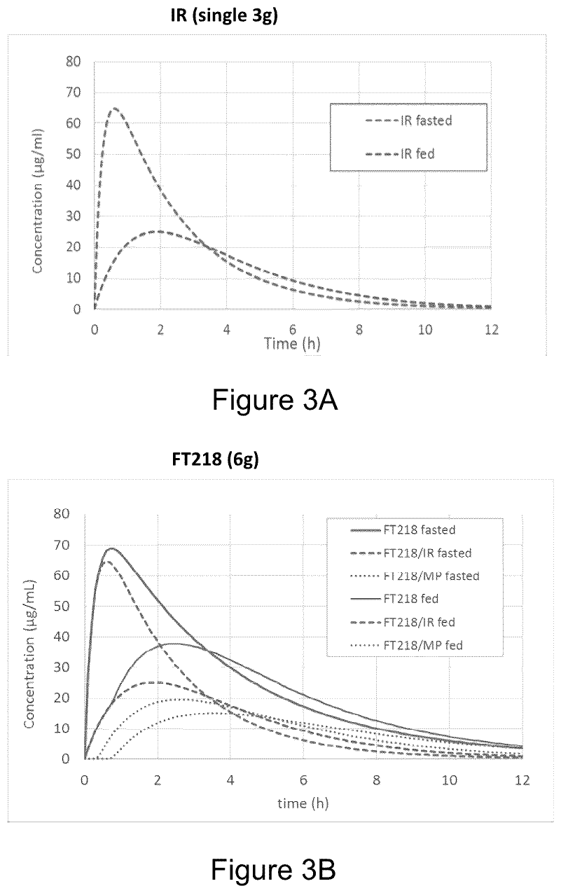

To illustrate the effect of food on FT218 and the IR and MR portions of FT218 for a 9 g dosage, concentration versus time curves were simulated. FIG. 3A shows the predicted negative effect of food on the IR portion of microparticles alone at a single 3 g dose. FIG. 3A shows a Cₘₐₓ(fed)/Cₘₐₓ(fasted)=40% and AUC(fed)/AUC(fasted)=70%. Food appears to have a similar impact on the IR microparticles of FT218 as on Xyrem®, as seen when comparing FIGS. 3A and 4A which shows concentration versus time curves for Xyrem in the fed and fasted state.

FIG. 3B shows that the predicted negative food effect is reduced in the MR portion of microparticles and in the full FT218 formulation as compared to the IR portion of microparticles. FIG. 3B shows a Cₘₐₓ(fed)/Cₘₐₓ(fasted)=55% and an AUC(fed)/AUC(fasted)=80% for FT218. Therefore, it appears that food may have no or low impact on MR microparticle absorption in the latter part of the gastrointestinal tract.

### Example 4. Comparison of Xyrem® and FT218 in the Fed, 2 hr Post Meal, and Fasted State

The effect of food on FT218 and Xyrem® is shown in FIGS. 4A and 4B. Data was digitized from the NDA dossier of Xyrem 21-196. FIG. 4A shows a concentration versus time curve for a 4.5 g single dose of Xyrem® in the fed state, the fasted state and 2 hours post meal. FIG. 4B shows a concentration versus time curve for a 6 g dose of FT218 in the fed state, the fasted state and 2 hours post meal. For Xyrem®, Cₘₐₓ and AUCs decrease as meal time is closer to administration, while Tₘₐₓ increases with food. In comparison, FT218 appears to demonstrate the same behavior as Xyrem®, but the impact of meal time seems reduced. Fed and 2 h post meal administration appear to have similar PK profiles (inter-study comparison). The derived PK parameters are summarized below (Table 4).

TABLE 4
Mean PK Parameters

| | Xyrem ® (4.5 g) | | | FT218 (6 g) | | |
|---|---|---|---|---|---|---|
| | fast | 2 h post meal^{a} | fed | fast | 2 h post meal | fed |
| Tₘₐₓ (h) | 0.75 | 1.25-1.14 | 2 | 0.53 [0.33-1] | 1.5 [0.33-3.5] | 1.5 [0.5-2.5] |
| Cₘₐₓ | 142 ± 34.2 | 88.9-83.0 | 60.1 ± 20.1 | 90.5 ± 15.8 | 64.6 ± 25.8 | 64.0 ± 17.5 |
| AUC_{0-inf} | 289 ± 109 | 241-233 | 188 ± 80 | 267 ± 85.2 | 273 ± 139 | 242 ± 88.2 |
| (μg/mL · h) | | | | | | |

^{a}means of study OMC-SXB-8 are presented.

### Example 5. Comparison of Xyrem® and FT218 in the Fed, 2 hr Post Meal, and Fasted State

To compare the effect of food on FT218 and Xyrem® in the fed state, 2 hours post-meal, and in the fasted state, the concentration versus time curves for a 3 g dose of Xyrem® administered twice and a single 6 g dose of FT218 were plotted together.

FIG. 5A shows expected concentration versus time curves for Xyrem® and FT218 in the fed state. Xyrem® in the fed state was extrapolated based on 4.5 g data from the NDA review of Xyrem® (study OMC-SXB-11) (linearized to the dose of 3 g and multiplied by a factor of correction of 0.7). As seen in FIG. 5A, Xyrem® risks of lack of efficacy during the first dosing, while the onset of efficacy is sooner for FT218 than Xyrem® and the Cₘₐₓ of FT218 is below the Cₘₐₓ for the 2nd peak of Xyrem®. Therefore, administering FT218 in the fed state may reduce safety concerns associated with administering Xyrem® in the fed state.

FIG. 5B shows expected concentration versus time curves for Xyrem® and FT218 administered two hours post-meal. The Xyrem® data is extrapolated from the PK FT218-1602 BA study. This study was a randomized, 2 treatments, 2 periods, 2 sequences cross-over study. The two treatments were: FT218 6 g administered 2 hours post evening meal and 2*3 Xyrem first dose administered 2 hours post evening meal, the 2^{nd} dose was administered 4 hours after the first dose. 28 subjects were included.

As seen in FIG. 5B, the Cₘₐₓ of FT218 is between the Cₘₐₓ of the first peak and the Cₘₐₓ of the second peak of Xyrem®. Therefore, administering FT218 at two hours post-meal may reduce safety concerns associated with Xyrem®. FIG. 5C shows expected concentration versus time curves for Xyrem® and FT218 administered in the fasted state. The Xyrem® data was simulated using numerical superposition.

The impact of the food effect of FT218 is likely reduced on Cₘₐₓ and AUCs compared to Xyrem® at the same dose based on food effect and dose proportionality data. The predictability of FT218 may be better than Xyrem®. Table 5 provides expected PK parameters for Xyrem® (2×3 g dose) and FT218 (6 g dose) in the fed state, two hours post-meal, and in the fasted state.

TABLE 5
Estimated PK Parameters

| | | | | | Cmax | | |
|---|---|---|---|---|---|---|---|
| | | | | | 2^{nd} | | |
| | Tmax (hr) | | Cmax 1^{st} peak | | peak | AUC0-inf | |
| | Xyrem | FT218 | Xyrem | FT218 | Xyrem | Xyrem | FT218 |
| Fed | >0.5 | 1.50 | <46.4 | 64.0 | ~70.9 | <259 | 242 |
| 2 h | 0.5 | 1.50 | 46.4 | 64.6 | 70.9 | 259 | 273 |
| post | | | | | | | |
| meal | | | | | | | |
| Fast | <0.5 | 0.53 | >46.4 | 90.5 | ~70.9 | >259 | 267 |

### Example 6. Comparison of Xyrem® and FT218 in the Fed, 2 hr Post Meal, and Fasted State

To compare the anticipated effect of food on FT218 and Xyrem® in the fed state, 2 hours post-meal, and in the fasted state, the concentration versus time curves for a 4.5 g dose of Xyrem® administered twice and a single 9 g dose of FT218 were plotted together.

FIG. 6A shows expected concentration versus time curves for Xyrem® and FT218 in the fed state. The FT218 data is from a dose proportionality study and the Xyrem® data is from the NDA dossier of Xyrem®.

The FT218 mean concentration-time profile is the observed data of the dose proportionality study at 9 g 2 hours post-meal. As seen in FIG. 6A, Xyrem® risks of lack of efficacy during the first dosing, while the onset of efficacy is sooner for FT218 than Xyrem® and the Cₘₐₓ of FT218 is below the Cₘₐₓ for the 2nd peak of Xyrem®. Therefore, administering FT218 in the fed state may reduce safety concerns associated with administering Xyrem® in the fed state.

FIG. 6B shows expected concentration versus time curves for Xyrem® and FT218 administered two hours post-meal. As seen in FIG. 6B, the Cₘₐₓ of FT218 is between the Cₘₐₓ of the first peak and the Cₘₐₓ of the second peak of Xyrem®. Therefore, administering FT218 at two hours post-meal may reduce safety concerns associated with Xyrem®. FIG. 6C shows expected concentration versus time curves for Xyrem® and FT218 administered in the fasted state. The Xyrem® and FT218 fasted data was extrapolated based on 4.5 g data (dose linearity assumption and multiplied by a factor of correction of 1.2).

The impact of the food effect of FT218 is likely reduced on Cₘₐₓ and AUCs compared to Xyrem® at the same dose based on food effect and dose proportionality data. The predictability of FT218 may be better than Xyrem®. Table 6 provides expected PK parameters for Xyrem® (2×4.5 g dose) and FT218 (9 g dose) in the fed state, two hours post-meal, and in the fasted state.

TABLE 6
Estimated PK Parameters

| | | | | | Cmax | | |
|---|---|---|---|---|---|---|---|
| | | | | | 2^{nd} | | |
| | Tmax (hr) | | Cmax 1^{st} peak | | peak | AUC0-inf | |
| | Xyrem | FT218 | Xyrem | FT218 | Xyrem | Xyrem | FT218 |
| Fed | 2 | >2 | 60.1 | <84.5 | ~142 | <518 | ~518 |
| 2 h | 1.17^{a} | 2 | 77.6 | 84.5 | 142 | 518 | ~518 |
| post | | | | | | | |
| meal | | | | | | | |
| Fast | 0.75 | <2 | 142 | >84.5 | ~142 | >518 | >518 |

### Example 7. Comparison of Xyrem® and FT218 PK Profiles for AUC and Cmax in the Fed State

To compare the effect on dosing time, AUC and Cₘₐₓ for 6 g of FT218 and Xyrem® were plotted over a period of time after eating a meal. FIG. 7A shows AUC for 6 g FT218 and 6 g Xyrem® versus time after a meal. FIG. 7B shows Cₘₐₓ for 6 g FT218 and 6 g Xyrem® versus time after a meal. The PK parameters were calculated on mean PK profiles. Circulating levels for FT218 may be more consistent than Xyrem® for each individual through the night, as overall the line shape profile for FT218 is more constant (fluctuation is reduced). In addition, circulating levels of GHB may be less impacted by meal (from fed to fast state). For example, taking FT218 during a meal, 1 hour, or 2 hours post-meal may lead to similar PK profiles. This may lead to fewer constraints for patients.

### Example 8. Pharmacokinetics and Dose Proportionality of FT218 for Once-Nightly Dosing

To assess the PK of FT218 given as a single dose of 4.5 g, 7.5 g and 9 g, compare PK parameters at the 3 doses, and estimate the dose proportionality, an open-label, single-dose, 3-sequential period study in 20 healthy volunteers was performed. Subjects received 3 separate single-dose (without titration) administrations of FT218 at bedtime, two hours post-evening meal, in a sequential order of 4.5 g, 7.5 g and 9 g with a minimum 7-day washout between doses. Dose proportionality between the three doses was assessed using the power method. Sensitivity analyses were performed using ANOVA.

Variability of concentrations of FT218 and twice-nightly sodium oxybate IR at 8 h and 10 h post-dose (when patients typically awaken) in the PK pilot and the present study were compared in terms of standard deviation.

The study was conducted in 20 healthy volunteers (12 males and 8 females). All subjects completed periods 1 (4.5 g) and 2 (7.5 g), while 12 subjects completed period 3 (9 g).

For the 3 doses, mean pharmacokinetics exhibited similar overall profiles with median Tₘₐₓ between 1.5 and 2 hours (FIG. 8). Mean Cₘₐₓ increased from 42.9 to 84.5 μg/mL across the increasing doses. Following Cₘₐₓ, blood levels gradually decreased overnight. Mean AUCᵢₙₜ was 191, 358 and 443 μg·h/mL for the 4.5, 7.5 and 9 g doses respectively. Mean concentrations at 8 hours were 4.8, 19.7 and 25.5 μg/mL for the 4.5, 7.5 and 9 g doses respectively.

Table 7 provides variability of concentrations at 8 h and 10 h post-dose for twice-nightly sodium oxybate and FT218 in the PK pilot and dose proportionality studies.

TABLE 7
Concentrations at 8 h and 10 h post-dose for twice-nightly
sodium oxybate and FT218

| | | FT218 | | |
|---|---|---|---|---|
| | Twice-nightly | | | Dose |
| | sodium | PK pilot study | | propor- |
| | oxybate IR | Part 1- | | tionality |
| | PK pilot study | Step 1 | Part 2 | study |
| PK parameter | 2 * 2.25 g | 4.5 g | 4.5 g | 4.5 g |
| | n = 15 | n = 14 | n = 12 | n = 20 |
| C₈ₕ mean ± SD | | | | |
| (μg/mL) | | | | |
| BQL set to missing | 9.24 ± 11.77 | 7.40 ± 5.88 | 6.27 ± 5.81 | 4.76 ± 5.0 |
| | (n = 14) | (n = 13) | | |
| BQL set to zero | 8.62 ± 11.59 | 6.87 ± 5.98 | 6.27 ± 5.81 | 4.76 ± 5.01 |
| C₁₀ₕ mean ± SD | | | | |
| (μg/mL) | | | | |
| BQL set to missing | 2.64 ± 3.84 | 1.21 ± 1.86 | 0.94 ± 0.55 | 0.73 ± 0.41 |
| | (n = 8) | (n = 8) | (n = 7) | (n = 9) |
| BQL set to zero | 1.41 ± 3.04 | 0.69 ± 1.50 | 0.55 ± 0.63 | 0.33 ± 0.46 |

BQL: concentration below quantitation limit.

Mean concentrations at 8 h and 1 Oh post-dose for FT218 are at least as low as twice-nightly sodium oxybate IR, regardless of the rule used to handle concentrations below quantitation limit (Table 7). Moreover, the variability of the concentrations was similar.

Applying the power method, the slope estimate for Cₘₐₓ was 1.02 and the confidence interval centered on 1.00 (90% CI: 0.76-1.28). For AUCᵢₙₜ, the estimate was 1.34 (90% CI: 1.19-1.48), indicating that the increase in the AUC is slightly more than proportional. These results were consistent with ANOVA sensitivity analysis results.

Thirteen subjects (65%) reported a total of 31 treatment emergent adverse events (TEAEs). 8 TEAEs (mainly headache 5/8) were experienced by 7/20 (35%) subjects during the 4.5 g period. 7 TEAEs (mainly gastrointestinal disorders 4/7) were experienced by 4/20 (20%) subjects during the 7.5 g period. 16 TEAEs (mainly gastrointestinal disorders 8/16) were experienced by 6/12 (50%) subjects during the 9 g period. One of these, a nervous system disorder (sedation) was a SAE. The intensity of TEAEs was judged severe for 2/31 TEAEs (both in 9 g period), moderate for 10/31 (4 in 4.5 g period, 3 in 7.5 g period and 3 in 9 g period) and mild for 19/31. All the TEAEs were resolved before the end of the study.

FT218 achieved blood-level profiles, when given at bedtime, consistent with a single CR dose. Dose proportionality was maintained for Cₘₐₓ across the dosage range. The safety profile was consistent with what is known for sodium oxybate and most AEs were mild to moderate in severity even without titration.

### Example 9. TEAEs for FT218 in the Fed and Fasted State

Table 8 provides a summary of the TEAEs in the fed and fasted states for a 6 g dose of FT218.

TABLE 8
Adverse Events with FT218

| | FT218 6 g single dose | FT218 6 g single dose |
|---|---|---|
| TEAE | Fasted (N = 16); n (%) | Fed (N = 15); n (%) |
| Somnolence | 13 (81.3) | 10 (66.7) |
| Dizziness | 7 (43.8) | 3 (20.0) |
| Nausea | 6 (37.5) | 1 (6.7) |
| Headache | 4 (25.0) | 2 (13.3) |
| Feeling Drunk | 4 (25.0) | 4 (26.7) |
| Vomiting | 3 (18.8) | 1 (6.7) |
| Fatigue | 3 (18.8) | 1 (6.7) |

As can be seen from Table 8, administration of 6 g FT218 in the fed state results in fewer TEAEs than 6 g FT218 administered in the fasted state.

Moreover, the pharmacokinetic-adverse event (AE) relationship for FT218 was evaluated. A total of 129 healthy volunteers received single doses of FT218 between 4.5-9 g. Six single-dose, randomized, crossover studies that assessed the pharmacokinetics of FT218 at 4.5, 6, 7.5 and 9 g in healthy volunteers were used. Lattice plots, “spaghetti” plots, and scatter plots of individual gamma hydroxybutyrate concentrations and indicators when AEs by system, organ, or class (SOC) were created to determine any PK-AE relationship.

Most AEs, specifically for the neurological and gastrointestinal SOC, occurred close to Tₘₐₓ, during the Cₘₐₓ period, which for FT218 was around 1.5-2 hours after dosing. These AEs were known AEs associated with sodium oxybate. There appeared to be no clear correlation between individual plasma GHB concentrations levels and AEs between subjects. Individual AEs were equally distributed above and below the mean population Cₘₐₓ and AUCᵢₙₜ for the dataset.

### Example 10. Soft Foods as Vehicles for Reconstitution

The objective of this example is to summarize the results from a set of experiments conducted to define the dissolution and pH testing of 4.5 g and 9 g Sodium Oxybate for Extended-Release Oral Suspension (FT218/Lumryz™) Stick Packs using soft foods as reconstitution vehicles. The dissolution study was performed to ensure the use of pudding, applesauce, or yogurt has no impact on the dissolution profile of this stick pack product. Dissolution profile testing and pH testing were evaluated.

### Materials, Equipment, and Data Reference

| Reference | | | | | Expiration |
|---|---|---|---|---|---|
| Standard | Source | OTW # | Lot # | Purity | Date |
| GBL | Sigma | 999-11 | MKCL7186 | 99.9% | May 5, 2027 |
| Sodium | Curia | 988-57 | US2060063 | 100.4% | Sep. 14, 2025 |
| Oxybate | | | | | |

| | | | Time point |
|---|---|---|---|
| | | | and |
| Test Articles | OTW # | Lot # | condition |
| 9 g Sodium Oxybate for | 2400580 | 4362023 | 36M 25° C./ |
| Extended-Release | | | 60% RH |
| Oral Suspension (FT218/Lumryz ™ ) | | | |
| Stick Packs | | | |
| 4.5 g Sodium Oxybate for | 2400577 | 4362012 | 36M 25° C./ |
| Extended-Release | | | 60% RH |
| Oral Suspension (FT218/Lumryz ™ ) | | | |
| Stick Packs | | | |

| | Reagent | | | | |
|---|---|---|---|---|---|
| | Grade or | | Part/ | | Expiration |
| Reagent/Consumable Name | Purity | Manufacturer | Catalog # | Lot # | Date |
| Water¹ | Milli-Q | Milli-Q | NA | NA | May 16, 2024² |
| | | (Catalent, | | | |
| | | WIN) | | | |
| Lemon Pudding | NA | Conagra | NA | 249334002 | Nov, 3, 2024 |
| Vanilla Yogurt | NA | WalMart Inc. | NA | 10486695 | Jun. 28, 2024 |
| Applesauce | NA | WalMart Inc. | NA | 3353C22 | Dec. 19, 2025 |
| Acetonitrile | HPLC | VWR | BDH83639 | 24B0762002 | Dec. 4, 2027 |
| Potassium Phosphate Monobasic | ACS | Millipore | PX1562 | 2023080219 | May 9, 2029 |
| o-Phosphoric Acid (85%) | HPLC | JT Baker | 0260 | 0000251959 | Jan, 27, 2025 |
| 5N Sodium Hydroxide | Reagent | Aqua | 8660 | 3090511 | Sep. 30, 2025 |
| | | Solutions | | | |
| 0.1N Hydrochloric Acid | USP | Life Cycle | 56095 | 223412645 | May 16, 2024² |
| 0.2 mm Nylon filter | NA | Millipore | GNWP04700 | 0000183147 | NA |
| 20 mm Cannula Filter | NA | QLA | FIL-020-01 | 220217-887- | NA |
| | | | | BJV | |

¹Expiration date is 7 Days from the date of collection.
²Date of the collection.

### Test Method and Specification

The current Catalent (Winchester, KY) test method 180032 (version 5.0, revision 04) for “Dissolution of Sodium Oxybate in 0.1 N Hl and pH 6.0 Buffer (pH shift)”, effective 9 Feb. 2024 was used for this dissolution study.

The current Catalent (Winchester, KY) specification SPEC-FG-210018 (version 6.0, revision 03) for “Sodium Oxybate for Extended-Release Oral Suspension (30% Coating Level)”, effective 16 Jun. 2022 was used for this dissolution and pH study.

### Product Specification Per SPEC-FG-210018 (Dissolution and pH Only):

Sodium Oxybate for Extended-Release Oral Suspension
Stick Packs

| | |
|---|---|
| pH | 5.5-5.9 |

Dissolution 0.1N HCl & pH 6.0 Buffer

| | |
|---|---|
| Stage 1 | 0.25 hours: 40-60% |
| | 2 hours: 42-62% |
| Stage 2 | 4 hours: ≥80% |

Minor adjustments were made to dissolution procedure as outlined below.

Adjusted Preparation Procedures (n = 6 per strength)

| | Media | | Settle | Total |
|---|---|---|---|---|
| Study | Volume | Sample Reconstitution | Time | Volume |
| Lemon | 825 mL | Initial volume to dilute stick | 5 minutes | 925 mL |
| Pudding | | pack: 50 mL pudding. Rinse | | |
| | | volume of container: 2 × 25 | | |
| | | mL milli-Q water. | | |
| Vanilla | 825 mL | Initial volume to dilute stick | 5 minutes | 925 mL |
| Yogurt | | pack: 50 mL yogurt. Rinse | | |
| | | volume of container: | | |
| | | 2 x 25 mL milli-Q water. | | |
| Applesauce | 825 mL | Initial volume to dilute stick | 5 minutes | 900 mL |
| | | pack: 50 mL applesauce. | | |
| | | Rinse volume of container: | | |
| | | 25 mL milli-Q water. | | |

Sample reconstitution with dosing cup line “A” is 50 mL fill line and line “B” is 25 mL rinse line. Perform the stage 2 pH adjustment for each study per Test Method 180032.

Sample preparation for the pH testing is outlined in the table below.

Preparation Procedures (n = 1 per strength)

| Study | Note pH | Sample Reconstitution | Post Sample |
|---|---|---|---|
| Milli-Q | Record the | Initial volume to dilute stick | Record the pH of |
| Water | pH of | pack: | each solution after |
| | each | 50 mL Milli-Q Water. | |
| Lemon | solution | Initial volume to dilute stick | the sample settled |
| Pudding | prior | pack: | for |
| | to sample | 50 mL pudding. | |
| Vanilla | addition | Initial volume to dilute stick | 5 minutes and 30 |
| Yogurt | | pack: | |
| | | 50 mL yogurt. | |
| Applesauce | | Initial volume to dilute stick | minutes |
| | | pack: | |
| | | 50 mL applesauce. | |

A single sample of each strength was prepared for each diluent/media.

### Results and Discussion

### System Suitability

| | | Sodium | | Pass |
|---|---|---|---|---|
| Parameter | Requirement | Oxybate Result | GBL Result | (Y/N) |
| Peak area in the mobile phase blank and | NMT 0.1% | ND | ND | Y |
| dissolution mediums against the average | | | | |
| area of the first 5 Standard A1 | | | | |
| Standard A1 First 5 % RSD | NMT 2.0% | 0.1 | 0.1-0.4 | Y |
| Standard A1 Over all % RSD | NMT 2.0% | 0.2-0.4 | 0.4-0.7 | Y |
| Response Factor Agreement First 5 | 98.0% - 102.0% | 100.8% -100.9% | 99.8% -99.9% | Y |
| Standard A1 and A2 | | | | |

### Dissolution Results

The soft foods samples were tested per protocol QCP-2024-025. The control sample were tested during stability testing at 36 Months 25° C./60% RH.

The dissolution of a sachet/stick pack of 4.5 g and 9 g FT218 was tested in lemon pudding, vanilla yogurt, and applesauce. The dissolution results are provided in the tables below.

As seen in the tables and figures, there was no impact on the dissolution profile of FT218 when mixed with a soft food. Having no impact on the dissolution profile means there is little to no dissolution of the modified release portion or the modified release portion does not dissolve within 5-30 min when mixed in the soft food. Alternatively, having no impact on the dissolution profile means the dissolution profile of the composition when suspended in a soft food is equivalent or bioequivalent to the same composition suspended in water (e.g. tap water or Milli-Q purified water).

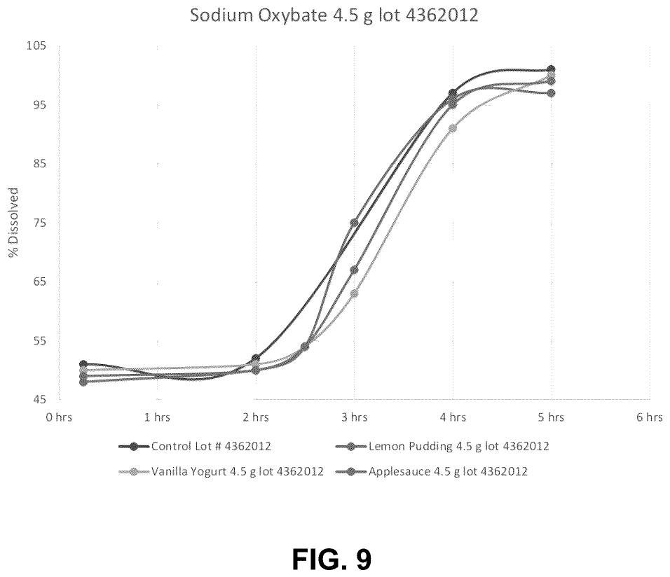

Table 9a and FIG. 9 shows dissolution results for a 4.5 g dose in lemon pudding.

TABLE 9a
Original Lemon Pudding-4.5 g
Original Lemon Pudding 4.5 g lot 4362012

| | Stage 1 | | Stage 2 | | | |
|---|---|---|---|---|---|---|
| Vessel | 15 min | 2.0 hrs | 2.5 hrs | 3.0 hrs | 4.0 hrs | 5.0 hrs |
| 1 | 46 | 50 | 55 | 79 | 98 | 99 |
| 2 | 46 | 50 | 57 | 83 | 99 | 100 |
| 3 | 45 | 47 | 54 | 78 | 94 | 96 |
| 4* | 46 | 49 | 50 | 53 | 60 | 66 |
| 5 | 48 | 50 | 57 | 83 | 98 | 100 |
| 6 | 46 | 48 | 56 | 82 | 97 | 97 |
| Min | 45 | 47 | 50 | 53 | 60 | 66 |
| Max | 48 | 50 | 57 | 83 | 99 | 100 |
| Mean | 46 | 49 | 55 | 76 | 91 | 93 |
| Std Dev | 0.8 | 1.0 | 2.7 | 11.6 | 15.5 | 13.1 |
| % RSD | 1.8 | 2.1 | 4.8 | 15.1 | 17.0 | 14.1 |

Low dissolution results most likely due to pH shift not performed completely for vessel number 4. The sample in the Lemon Pudding was retested ensuring proper pH shift. The retest results were utilized for the comparative tables and graph.

Table 9b and FIG. 9 shows dissolution results for a 4.5 g dose in lemon pudding.

TABLE 9b
Retest Lemon Pudding-4.5 g
Retest Lemon Pudding 4.5 g lot 4362012

| | Stage 1 | | Stage 2 | | | |
|---|---|---|---|---|---|---|
| Vessel | 15 min | 2.0 hrs | 2.5 hrs | 3.0 hrs | 4.0 hrs | 5.0 hrs |
| 1 | 47 | 49 | 53 | 72 | 94 | 96 |
| 2 | 46 | 49 | 52 | 73 | 94 | 94 |
| 3 | 48 | 50 | 54 | 77 | 96 | 97 |
| 4 | 49 | 51 | 55 | 75 | 97 | 98 |
| 5 | 49 | 52 | 56 | 77 | 98 | 100 |
| 6 | 49 | 51 | 55 | 75 | 97 | 98 |
| Min | 46 | 49 | 52 | 72 | 94 | 94 |
| Max | 49 | 52 | 56 | 77 | 98 | 100 |
| Mean | 48 | 50 | 54 | 75 | 96 | 97 |
| Std Dev | 1.5 | 1.4 | 1.4 | 2.1 | 1.8 | 2.1 |
| % RSD | 3.1 | 2.7 | 2.6 | 2.8 | 1.8 | 2.1 |

Table 10 and FIG. 9 shows dissolution results for a 4.5 g dose in vanilla yogurt.

TABLE 10
Vanilla Yogurt-4.5 g
Vanilla Yogurt 4.5 g lot 4362012

| | Stage 1 | | Stage 2 | | | |
|---|---|---|---|---|---|---|
| Vessel | 15 min | 2.0 hrs | 2.5 hrs | 3.0 hrs | 4.0 hrs | 5.0 hrs |
| 1 | 51 | 52 | 55 | 66 | 94 | 101 |
| 2 | 51 | 51 | 54 | 63 | 90 | 100 |
| 3 | 51 | 51 | 54 | 64 | 94 | 100 |
| 4 | 51 | 51 | 54 | 63 | 92 | 100 |
| 5 | 51 | 51 | 53 | 62 | 90 | 101 |
| 6 | 49 | 50 | 51 | 59 | 85 | 96 |
| Min | 49 | 50 | 51 | 59 | 85 | 96 |
| Max | 51 | 52 | 55 | 66 | 94 | 101 |
| Mean | 50 | 51 | 54 | 63 | 91 | 100 |
| Std Dev | 0.9 | 0.8 | 1.2 | 2.3 | 3.4 | 1.7 |
| % RSD | 1.8 | 1.5 | 2.2 | 3.6 | 3.7 | 1.7 |

Table 11 and FIG. 9 shows dissolution results for a 4.5 g dose in applesauce.

TABLE 11
Applesauce-4.5 g
Applesauce 4.5 g lot 4362012

| | Stage 1 | | Stage 2 | | | |
|---|---|---|---|---|---|---|
| Vessel | 15 min | 2.0 hrs | 2.5 hrs | 3.0 hrs | 4.0 hrs | 5.0 hrs |
| 1 | 49 | 50 | 53 | 65 | 95 | 100 |
| 2 | 49 | 50 | 54 | 66 | 96 | 99 |
| 3 | 49 | 50 | 53 | 68 | 95 | 99 |
| 4 | 49 | 50 | 54 | 67 | 95 | 99 |
| 5 | 50 | 51 | 55 | 70 | 96 | 100 |
| 6 | 50 | 50 | 54 | 64 | 93 | 97 |
| Min | 49 | 50 | 53 | 64 | 93 | 97 |
| Max | 50 | 51 | 55 | 70 | 96 | 100 |
| Mean | 49 | 50 | 54 | 67 | 95 | 99 |
| Std Dev | 0.4 | 0.5 | 0.6 | 2.0 | 1.2 | 1.1 |
| % RSD | 0.9 | 1.0 | 1.1 | 3.0 | 1.3 | 1.1 |

Table 12 and FIG. 9 shows dissolution results for a 4.5 g control. Table 12. Control

TABLE 12
Control
Control lot 4362012

| | Stage 1 | | Stage 2 | |
|---|---|---|---|---|
| Vessel | 15 min | 2.0 hrs | 4.0 hrs | 5.0 hrs |
| 1 | 51 | 52 | 98 | 102 |
| 2 | 51 | 52 | 98 | 102 |
| 3 | 52 | 53 | 98 | 102 |
| 4 | 52 | 53 | 99 | 102 |
| 5 | 51 | 52 | 96 | 102 |
| 6 | 50 | 50 | 94 | 98 |
| Min | 50 | 50 | 94 | 98 |
| Max | 52 | 53 | 99 | 102 |
| Mean | 51 | 52 | 97 | 101 |
| Std Dev | 0.8 | 0.9 | 1.9 | 1.7 |
| % RSD | 1.6 | 1.7 | 2.0 | 1.7 |

Table 13 shows a dissolution summary for a 4.5 g dose in control conditions and soft foods as a vehicle for reconstitution.

TABLE 13
Dissolution Summary 4.5 g
Dissolution Summary for lot 4362012 4.5 g in control conditions and soft foods

| | | | | *Lemon | Vanilla | |
|---|---|---|---|---|---|---|
| | | Control | | Pudding | Yogurt | Applesauce |
| Condition/Soft food | | SPEC-FG-210018 | | | | |
| Specification | | (version 6.0, revision 03) | | N/A | | |
| Stage 1 | 0.25 | Min | 50 | 46 | 49 | 49 |
| Stage 2 | hours: | Max | 52 | 49 | 51 | 50 |
| | 40-60% | Mean | 51 | 48 | 50 | 49 |
| | | % RSD | 1.6 | 3.1 | 1.8 | 0.9 |
| | 2.0 | Min | 50 | 49 | 50 | 50 |
| | hours: | Max | 53 | 52 | 52 | 51 |
| | 42-62% | Mean | 52 | 50 | 51 | 50 |
| | | % RSD | 1.7 | 2.7 | 1.5 | 1.0 |
| | 4.0 | Min | 94 | 94 | 85 | 93 |
| | hours: | Max | 99 | 98 | 94 | 96 |
| | ≥80% | Mean | 97 | 96 | 91 | 95 |
| | | % RSD | 2.0 | 1.8 | 3.7 | 1.3 |
| | ** 5.0 | Min | 98 | 94 | 96 | 97 |
| | hours | Max | 102 | 100 | 101 | 100 |
| | | Mean | 101 | 97 | 100 | 99 |
| | | % RSD | 1.7 | 2.1 | 1.7 | 1.1 |

*Lemon Pudding retest results
** 5.0 hours is not part of specification

### Acceptance Criteria

The dissolution profile of each sample was reported, as seen in summary table 13. The dissolution release profile for the original low dissolution results in lemon pudding had an atypical result for vessel #4, most likely due to a pH shift that was not performed completely for vessel number 4. The lemon pudding sample was retested ensuring proper pH shift during analysis. The retest results were used for the comparison to the control results. The control results were obtained from the 36-month 25° C./60% RH stability testing of stability protocol QCP-2021-001.

Dissolution release profile mean in all soft foods as vehicles for reconstitution met the specification for conditions. The lemon pudding samples (75%) released faster than the other sample matrix (63% and 67%) at the 3.0 hour time point, but recovered similarly at the two later points (4 and 5 hours). Additionally, the profiles of the alternate food vehicles are similar to that of the control sample.

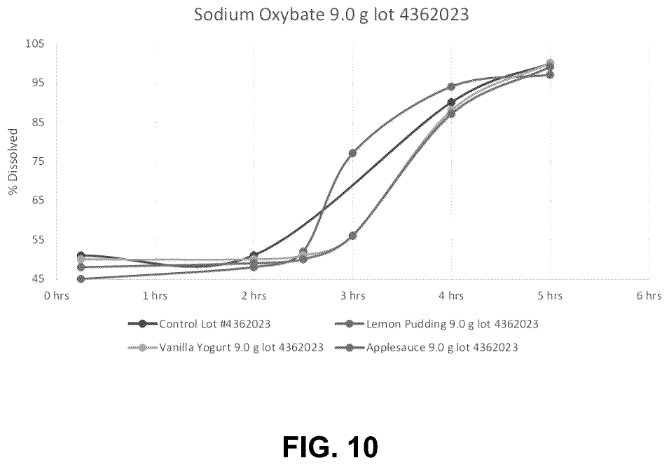

Table 14 and FIG. 10 shows dissolution results for a 9.0 g dose in lemon pudding.

TABLE 14
Lemon Pudding-9.0 g
Lemon Pudding 9.0 g lot 4362023

| | Stage 1 | | Stage 2 | | | |
|---|---|---|---|---|---|---|
| Vessel | 15 min | 2.0 hrs | 2.5 hrs | 3.0 hrs | 4.0 hrs | 5.0 hrs |
| 1 | 43 | 48 | 53 | 81 | 100 | 102 |
| 2 | 45 | 47 | 50 | 72 | 91 | 95 |
| 3 | 49 | 50 | 52 | 76 | 94 | 96 |
| 4 | 44 | 48 | 53 | 86 | 99 | 100 |
| 5 | 48 | 50 | 52 | 70 | 90 | 94 |
| 6 | 42 | 47 | 50 | 75 | 93 | 95 |
| Min | 42 | 47 | 50 | 70 | 90 | 94 |
| Max | 49 | 50 | 53 | 86 | 100 | 102 |
| Mean | 45 | 48 | 52 | 77 | 94 | 97 |
| Std Dev | 2.5 | 1.3 | 1.3 | 5.8 | 4.1 | 3.2 |
| % RSD | 5.5 | 2.6 | 2.5 | 7.6 | 4.3 | 3.3 |

Table 15 and FIG. 10 shows dissolution results for a 9.0 g dose in vanilla yogurt.

TABLE 15
Vanilla Yogurt-9.0 g
Vanilla Yogurt 9.0 g lot 4362023

| | Stage 1 | | Stage 2 | | | |
|---|---|---|---|---|---|---|
| Vessel | 15 min | 2.0 hrs | 2.5 hrs | 3.0 hrs | 4.0 hrs | 5.0 hrs |
| 1 | 49 | 50 | 51 | 54 | 83 | 100 |
| 2 | 49 | 49 | 50 | 55 | 89 | 99 |
| 3 | 50 | 51 | 52 | 58 | 92 | 102 |
| 4 | 52 | 50 | 51 | 58 | 92 | 101 |
| 5 | 50 | 50 | 51 | 55 | 88 | 100 |
| 6 | 49 | 49 | 51 | 55 | 86 | 100 |
| Min | 49 | 49 | 50 | 54 | 83 | 97 |
| Max | 52 | 51 | 52 | 58 | 92 | 102 |
| Mean | 50 | 50 | 51 | 56 | 88 | 100 |
| Std Dev | 1.4 | 0.6 | 0.8 | 1.5 | 3.5 | 1.7 |
| % RSD | 2.7 | 1.3 | 1.5 | 2.7 | 4.0 | 1.7 |

Table 16 and FIG. 10 shows dissolution results for a 9.0 g dose in applesauce.

TABLE 16
Applesauce-9.0 g
Applesauce 9.0 g lot 4362023

| | Stage 1 | | Stage 2 | | | |
|---|---|---|---|---|---|---|
| Vessel | 15 min | 2.0 hrs | 2.5 hrs | 3.0 hrs | 4.0 hrs | 5.0 hrs |
| 1 | 50 | 50 | 51 | 55 | 86 | 100 |
| 2 | 47 | 48 | 50 | 54 | 85 | 96 |
| 3 | 48 | 49 | 50 | 56 | 89 | 99 |
| 4 | 48 | 49 | 51 | 56 | 88 | 98 |
| 5 | 48 | 48 | 50 | 54 | 85 | 97 |
| 6 | 48 | 49 | 51 | 61 | 90 | 102 |
| Min | 47 | 48 | 50 | 54 | 85 | 96 |
| Max | 50 | 50 | 51 | 61 | 90 | 102 |
| Mean | 48 | 49 | 50 | 56 | 87 | 99 |
| Std Dev | 1.0 | 0.7 | 0.7 | 2.6 | 2.1 | 2.2 |
| % RSD | 2.0 | 1.4 | 1.4 | 4.6 | 2.4 | 2.2 |

Table 17 and FIG. 10 shows dissolution results for a 9.0 g control. Table 17. Control

TABLE 17
Control
Control lot 4362023

| | Stage 1 | | Stage 2 | |
|---|---|---|---|---|
| Vessel | 15 min | 2.0 hrs | 4.0 hrs | 5.0 hrs |
| 1 | 50 | 50 | 92 | 99 |
| 2 | 53 | 53 | 94 | 104 |
| 3 | 49 | 49 | 87 | 97 |
| 4 | 52 | 52 | 89 | 102 |
| 5 | 50 | 50 | 85 | 98 |
| 6 | 51 | 51 | 91 | 101 |
| Min | 49 | 49 | 85 | 97 |
| Max | 53 | 53 | 94 | 104 |
| Mean | 51 | 51 | 90 | 100 |
| Std Dev | 1.4 | 1.4 | 3.2 | 2.7 |
| % RSD | 2.7 | 2.7 | 3.5 | 2.7 |

Table 18 shows a dissolution summary for a 9.0 g dose in control conditions and soft foods as a vehicle for reconstitution.

TABLE 18
Dissolution Summary 4.5 g
Dissolution Summary for lot 4362023 9.0 g in control conditions and soft foods

| | | | | Lemon | Vanilla | |
|---|---|---|---|---|---|---|
| | | Control | | Pudding | Yogurt | Applesauce |
| Condition/Soft food | | SPEC-FG-210018 | | | | |
| Specification | | (version 6.0, revision 03) | | N/A | | |
| Stage 1 | 0.25 | Min | 49 | 42 | 49 | 47 |
| Stage 2 | hours: | Max | 53 | 49 | 52 | 50 |
| | 40-60% | Mean | 51 | 45 | 50 | 48 |
| | | % RSD | 2.7 | 5.5 | 2.7 | 2.0 |
| | 2.0 | Min | 49 | 47 | 49 | 48 |
| | hours: | Max | 53 | 50 | 51 | 50 |
| | 42-62% | Mean | 51 | 48 | 50 | 49 |
| | | % RSD | 2.7 | 2.6 | 1.3 | 1.4 |
| | 4.0 | Min | 85 | 90 | 83 | 85 |
| | hours: | Max | 94 | 100 | 92 | 90 |
| | ≥80% | Mean | 90 | 94 | 88 | 87 |
| | | % RSD | 3.5 | 4.3 | 4.0 | 2.4 |
| | ** 5.0 | Min | 97 | 94 | 97 | 96 |
| | hours | Max | 104 | 102 | 102 | 102 |
| | | Mean | 100 | 97 | 100 | 99 |
| | | % RSD | 2.7 | 3.3 | 1.7 | 2.2 |

** 5.0 hours is not part of specification

### Acceptance Criteria

The dissolution profile of each sample was reported for information only (see above summary table).

Dissolution release profile in all soft foods as vehicles for reconstitution also met the specification for control conditions. Dissolution release profile mean in all soft foods as vehicles for reconstitution met the specification for conditions. The lemon samples (77%) release faster than the other sample matrix (56% and 56%) at the 3.0 hour time point, but recovers similarly at the two later points (4 and 5 hours). Additionally, the profiles of the alternate food vehicles are similar to that of the control sample.

### pH Results

The soft foods samples were performed per protocol QCP-2024-025.

The pH of the soft food vehicles/media before and after each sample preparation was measured and reported. The pH was further measured after adding the composition to the vehicle/diluent for both 4.5 g and 9 g dosages. The pH results are provided in the tables below.

TABLE 19
pH Results
pH Results Summary of samples in control conditions and soft
foods as vehicles for reconstitution

| | Pre- | | |
|---|---|---|---|
| Condition/Soft | Sample | Post-Sample Addition | |
| food | Addition | 4362012 (4.5 g) | 4362023 (9.0 g) |
| Control | N/A | 5.7 | 5.8 |
| Pudding | 3.6 | 5.3 | 5.5 |
| Yogurt | 4.1 | 5.3 | 5.5 |
| Applesauce | 3.6 | 5.3 | 5.5 |

Throughout this application, various publications are referenced. The disclosures of these publications in their entireties are hereby incorporated by reference into this application in order to more fully describe the state of the art to which this invention pertains. It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Numerous examples are provided herein to enhance the understanding of the present disclosure. In this regard, a specific set of statements are provided below characterizing various examples of oral pharmaceutical compositions and methods of treatment described herein.

Statement 1: An oral pharmaceutical composition for the treatment of narcolepsy, cataplexy, or excessive daytime sleepiness comprising gamma-hydroxybutyrate in a unit dose suitable for administration less than two hours after eating.

Statement 2: The oral pharmaceutical composition of Statement 1, wherein the composition is suitable for administrating with food, immediately after eating, up to 30 minutes after eating, up to 1 hour after eating, up to 1.5 hours after eating, or up to 2 hours after eating.

Statement 3: The oral pharmaceutical composition of Statements 1 or 2, wherein the composition is administered once daily.

Statement 4: The oral pharmaceutical composition of any one of the preceding Statements, wherein the composition is suitable for administration in the evening.

Statement 5: The oral pharmaceutical composition of any one of Statements 1-3, wherein the composition is suitable for administration in the morning.

Statement 6: The oral pharmaceutical composition of any one of the preceding Statements, wherein the composition comprises gamma-hydroxybutyrate in an extended release or delayed release formulation.

Statement 7: The oral pharmaceutical composition of any one of Statements 1-5, wherein the composition comprises gamma-hydroxybutyrate in a modified release formulation.

Statement 8: The oral pharmaceutical composition of any one of the preceding Statements, wherein the composition provides a substantially similar fed state PK profile and 2 hour post meal administration PK profile.

Statement 9: The oral pharmaceutical composition of any one of the preceding Statements, wherein the composition provides a mean AUCᵢₙₜ when administered less than two hour after eating that is 50%-120% of the mean AUCᵢₙₜ when the composition is administered at least two hours after eating.

Statement 10: The oral pharmaceutical composition of any one of the preceding Statements, wherein the composition provides a mean AUCᵢₙₜ when administered less than two hour after eating that is 50%-120% of the mean AUCᵢₙₜ when the composition is administered while fasting.

Statement 11: The oral pharmaceutical composition of any one of the preceding Statements, wherein the composition provides a mean AUCᵢₙₜ when administered less than two hour after eating that is 80%-95% of the mean AUCᵢₙₜ when the composition is administered while fasting.

Statement 12: The oral pharmaceutical composition of any one of the preceding Statements, wherein the composition provides a mean AUCᵢₙₜ when administered less than two hour after eating that is 85%-90% of the mean AUCᵢₙₜ when the composition is administered while fasting.

Statement 13: The oral pharmaceutical composition of any one of the preceding Statements, wherein the composition provides a mean Cₘₐₓ when administered less than two hour after eating that is 50%-120% of the mean Cₘₐₓ when the composition is administered at least two hours after eating.

Statement 14: The oral pharmaceutical composition of any one of the preceding Statements, wherein the composition provides a mean Cₘₐₓ when administered less than two hour after eating that is 50%-120% of the mean Cₘₐₓ when the composition is administered while fasting.

Statement 15: The oral pharmaceutical composition of any one of the preceding Statements, wherein the composition provides a mean Cₘₐₓ when administered less than two hour after eating that is 55%-80% of the mean Cₘₐₓ when the composition is administered while fasting.

Statement 16: The oral pharmaceutical composition of any one of the preceding Statements, wherein the composition provides a mean Cₘₐₓ when administered less than two hour after eating that is 60%-75% of the mean Cₘₐₓ when the composition is administered while fasting.

Statement 17: The oral pharmaceutical composition of any one of the preceding Statements, wherein the composition provides a Cₘₐₓ that is dose proportional.

Statement 18: The oral pharmaceutical composition of any one of the preceding Statements, wherein the composition provides no dose dumping.

Statement 19: The oral pharmaceutical composition of any one of the preceding Statements, wherein there is no significant reduction in safety or efficacy to a patient following administration.

Statement 20: The oral pharmaceutical composition of any one of the preceding Statements, wherein the composition provides an AUCᵢₙₜ bioequivalent to an AUCᵢₙₜ of an equal dose of immediate release liquid solution of sodium oxybate administered at t₀ and t₄ₕ in equally divided doses at least two hours after eating.

Statement 21: The oral pharmaceutical composition of any one of the preceding Statements, wherein the composition provides a Cₘₐₓ that is less than the Cₘₐₓ of an equal dose of immediate release liquid solution of sodium oxybate administered at t₀ and t₄ₕ in equally divided doses at least two hours after eating.

Statement 22: The oral pharmaceutical composition of any one of the preceding Statements, wherein the composition provides a Cₘₐₓ that is less than the Cₘₐₓ of an equal dose of immediate release liquid solution of sodium oxybate administered at t₀ and t₄ₕ in equally divided doses less than two hours after eating.

Statement 23: The oral pharmaceutical composition of any one of the preceding Statements, wherein the composition provides a Cₘₐₓ that is 10-60% less than the Cₘₐₓ of an equal dose of immediate release liquid solution of sodium oxybate administered at t₀ and t₄ₕ in equally divided doses at least two hours after eating.

Statement 24: The oral pharmaceutical composition of any one of the preceding Statements, wherein the composition provides a Cₘₐₓ that is 10-60% less than the Cₘₐₓ of an equal dose of immediate release liquid solution of sodium oxybate administered at t₀ and t₄ₕ in equally divided doses less than two hours after eating.

Statement 25: The oral pharmaceutical composition of any one of the preceding Statements, wherein the composition provides a change in Cₘₐₓ between when the composition is administered at least two hours after eating and when the composition is administered less than two hours after eating that is 10-60% less than the change in Cₘₐₓ of an equal dose of immediate release liquid solution of sodium oxybate administered at t₀ and t₄ₕ in equally divided doses at least two hours after eating.

Statement 26: The oral pharmaceutical composition of any one of the preceding Statements, wherein the composition provides a change in Cₘₐₓ between when the composition is administered at least two hours after eating and when the composition is administered less than two hours after eating that is 10-60% less than the change in Cₘₐₓ of an equal dose of immediate release liquid solution of sodium oxybate administered at t₀ and t₄ₕ in equally divided doses less than two hours after eating.

Statement 27: The oral pharmaceutical composition of any one of the preceding Statements, wherein the composition provides an AUC that is more dose proportional than the AUC of an equal dose of immediate release liquid solution of sodium oxybate administered at t₀ and t₄ₕ in equally divided doses at least two hours after eating.

Statement 28: The oral pharmaceutical composition of any one of the preceding Statements, wherein the composition provides an AUC that is more dose proportional than the AUC of an equal dose of immediate release liquid solution of sodium oxybate administered at t₀ and t₄ₕ in equally divided doses less than two hours after eating.

Statement 29: The oral pharmaceutical composition of any one of Statements 20-28, wherein the equal dose of immediate release liquid solution of sodium oxybate administered at t₀ and t₄ₕ in equally divided doses is Xyrem®.

Statement 30: An oral pharmaceutical composition for the treatment of narcolepsy, cataplexy, or excessive daytime sleepiness comprising gamma-hydroxybutyrate in a unit dose, wherein the absorption of the gamma-hydroxybutyrate in the gastro-intestinal tract is not substantially changed by the presence of food.

Statement 31: An oral pharmaceutical composition comprising gamma-hydroxybutyrate in a unit dose suitable for administration less than two hours after eating.

Statement 32: The oral pharmaceutical composition of Statement 31, wherein the composition comprises gamma-hydroxybutyrate in a modified release formulation.

Statement 33: The oral pharmaceutical composition of Statements 31 or 32, wherein the formulation is administered during, immediately after, up to 30 minutes after eating, up to 1 hour after eating, up to 1.5 hours after eating, or up to 2 hours after eating.

Statement 34: The oral pharmaceutical composition of any one of Statements 31-33, wherein the composition is suitable for once-daily administration.

Statement 35: The oral pharmaceutical composition of any one of Statements 31-34, wherein the composition is suitable for administration in the evening.

Statement 36: The oral pharmaceutical composition of any one of Statements 31-34, wherein the composition is suitable for administration in the morning.

Statement 37: The oral pharmaceutical composition of any one of Statements 31-36, wherein the composition is effective to induce sleep for at least eight consecutive hours.

Statement 38: The oral pharmaceutical composition of any one of Statements 31-37, wherein a 6 g dose of the composition administered less than two hours after eating has a mean AUCᵢₙₜ of greater than 230 hr*μg/mL, and a mean Cₘₐₓ that is from 50% to 140% of the mean Cₘₐₓ provided by an equal dose of immediate release liquid solution of sodium oxybate administered at t₀ and t₄ₕ in equally divided doses at least two hours after eating.

Statement 39: The oral pharmaceutical composition of Statement 38, wherein the mean Cₘₐₓ is from 80% to 140% of the mean Cₘₐₓ provided by an equal dose of immediate release liquid solution of sodium oxybate administered at t₀ and t₄ₕ in equally divided doses at least two hours after eating.

Statement 40: The oral pharmaceutical composition of Statement 38, wherein the mean Cₘₐₓ is from 100% to 150% of the of the mean Cₘₐₓ of a first peak of the equal dose of immediate release liquid solution of sodium oxybate administered at t₀ and t₄ₕ in equally divided doses at least two hours after eating.

Statement 41: The oral pharmaceutical composition of Statement 38, wherein the mean Cₘₐₓ is from 80% to 100% of the of the mean Cₘₐₓ of a second peak of the equal dose of immediate release liquid solution of sodium oxybate administered at t₀ and t₄ₕ in equally divided doses at least two hours after eating.

Statement 42: The oral pharmaceutical composition of Statement 38, wherein the mean Cₘₐₓ is about 50 μg/mL to about 82 μg/mL.

Statement 43: The oral pharmaceutical composition of Statement 42, wherein the mean Cₘₐₓ is about 64 μg/mL.

Statement 44: The oral pharmaceutical composition of Statement 38, wherein the mean AUCᵢₙₜ is about 242 hr*μg/mL when the formulation is administered about 30 minutes after eating.

Statement 45: The oral pharmaceutical composition of Statement 38, wherein the mean AUC_{inf} is about 273 hr*μg/mL when the formulation is administered about 2 hours after eating.

Statement 46: A method of treating narcolepsy and associated disorders and symptoms in a patient in need thereof comprising: administering an oral pharmaceutical composition comprising gamma-hydroxybutyrate less than two hours after eating.

Statement 47: The method of Statement 46, wherein the composition is administered once-daily.

Statement 48: The method of Statement 47, wherein the composition is administered after eating in the evening.

Statement 49: The method of Statement 47, wherein the composition is administered after eating in the morning.

Statement 50: The method of any one of Statements 46-49, wherein the composition is administered during, immediately after eating, up to 30 minutes after eating, up to 1 hour after eating, up to 1.5 hours after eating, or up to 2 hours after eating.

Statement 51: The method of any one of Statements 46-50, wherein the composition is effective to induce sleep for at least six consecutive hours.

Statement 52: The method of any one of Statements 46-51, wherein the composition is effective to induce sleep for at least eight consecutive hours.

Statement 53: The method of any one of Statements 46-52, wherein the composition is effective to induce sleep for at least ten consecutive hours.

Statement 54: The method of any one of Statements 46-53, wherein the composition comprises gamma-hydroxybutyrate in an extended release or delayed release formulation.

Statement 55: The method of any one of Statements 46-53, wherein the composition comprises gamma-hydroxybutyrate in a modified release formulation.

Statement 56: The method of any one of Statements 46-55, wherein the composition provides a substantially similar fed state PK profile and 2 hour post meal administration PK profile.

Statement 57: The method of any one of Statements 46-56, wherein the composition provides a mean AUCᵢₙₜ when administered less than two hour after eating that is 50%-120% of the mean AUCᵢₙₜ when the composition is administered at least two hours after eating.

Statement 58: The method of any one of Statements 46-57, wherein the composition provides a mean AUCᵢₙₜ when administered less than two hour after eating that is 50%-120% of the mean AUCᵢₙₜ when the composition is administered while fasting.

Statement 59: The method of any one of Statements 46-58, wherein the composition provides a mean AUCᵢₙₜ when administered less than two hour after eating that is 80%-95% of the mean AUCᵢₙₜ when the composition is administered while fasting.

Statement 60: The method of any one of Statements 46-59, wherein the composition provides a mean AUCᵢₙₜ when administered less than two hour after eating that is 85%-90% of the mean AUCᵢₙₜ when the composition is administered while fasting.

Statement 61: The method of any one of Statements 46-60, wherein the composition provides a mean Cₘₐₓ when administered less than two hour after eating that is 50%-120% of the mean Cₘₐₓ when the composition is administered at least two hours after eating.

Statement 62: The method of any one of Statements 46-61, wherein the composition provides a mean Cₘₐₓ when administered less than two hour after eating that is 50%-120% of the mean Cₘₐₓ when the composition is administered while fasting.

Statement 63: The method of any one of Statements 46-62, wherein the composition provides a mean Cₘₐₓ when administered less than two hour after eating that is 55%-80% of the mean Cₘₐₓ when the composition is administered while fasting.

Statement 64: The method of any one of Statements 46-63, wherein the composition provides a mean Cₘₐₓ when administered less than two hour after eating that is 60%-75% of the mean Cₘₐₓ when the composition is administered while fasting.

Statement 65: The method of any one of Statements 46-64, wherein the composition provides a Cₘₐₓ that is dose proportional.

Statement 66: The method of any one of Statements 46-65, wherein the composition provides no dose dumping.

Statement 67: The method of any one of Statements 46-66, wherein there is no significant reduction in safety or efficacy to a patient following administration.

Statement 68: The method of any one of Statements 46-67, wherein the composition provides an AUCᵢₙₜ bioequivalent to an AUCᵢₙₜ of an equal dose of immediate release liquid solution of sodium oxybate administered at t₀ and t₄ₕ in equally divided doses at least two hours after eating.

Statement 69: The method of any one of Statements 46-68, wherein the composition provides a Cₘₐₓ that is less than the Cₘₐₓ of an equal dose of immediate release liquid solution of sodium oxybate administered at t₀ and t₄ₕ in equally divided doses at least two hours after eating.

Statement 70: The method of any one of Statements 46-69, wherein the composition provides a Cₘₐₓ that is less than the Cₘₐₓ of an equal dose of immediate release liquid solution of sodium oxybate administered at t₀ and t₄ₕ in equally divided doses less than two hours after eating.

Statement 71: The method of any one of Statements 46-70, wherein the composition provides a Cₘₐₓ that is 10-60% less than the Cₘₐₓ of an equal dose of immediate release liquid solution of sodium oxybate administered at t₀ and t₄ₕ in equally divided doses at least two hours after eating.

Statement 72: The method of any one of Statements 46-71, wherein the composition provides a Cₘₐₓ that is 10-60% less than the Cₘₐₓ of an equal dose of immediate release liquid solution of sodium oxybate administered at t₀ and t₄ₕ in equally divided doses less than two hours after eating.

Statement 73: The method of any one of Statements 46-72, wherein the composition provides a change in Cₘₐₓ between when the composition is administered at least two hours after eating and when the composition is administered less than two hours after eating that is 10-60% less than the change in Cₘₐₓ of an equal dose of immediate release liquid solution of sodium oxybate administered at t₀ and t₄ₕ in equally divided doses at least two hours after eating.

Statement 74: The method of any one of Statements 46-73, wherein the composition provides a change in Cₘₐₓ between when the composition is administered at least two hours after eating and when the composition is administered less than two hours after eating that is 10-60% less than the change in Cₘₐₓ of an equal dose of immediate release liquid solution of sodium oxybate administered at t₀ and t₄ₕ in equally divided doses less than two hours after eating.

Statement 75: The method of any one of Statements 46-74, wherein the composition provides an AUC that is more dose proportional than the AUC of an equal dose of immediate release liquid solution of sodium oxybate administered at t₀ and t₄ₕ in equally divided doses at least two hours after eating.

Statement 76: The method of any one of Statements 46-75, wherein the equal dose of immediate release liquid solution of sodium oxybate administered at t₀ and t₄ₕ in equally divided doses is Xyrem®.

Statement 77: The method of any one of Statements 46-76, wherein a 6 g dose of the composition administered has been shown to achieve a mean AUCᵢₙₜ of greater than 230 hr*μg/mL, and a mean Cₘₐₓ that is from 50% to 140% of the mean Cₘₐₓ provided by an equal dose of immediate release liquid solution of sodium oxybate administered at t₀ and t₄ₕ in equally divided doses at least two hours after eating.

Statement 78: The method of Statement 77, wherein the mean Cₘₐₓ is from 80% to 140% of the mean Cₘₐₓ provided by an equal dose of immediate release liquid solution of sodium oxybate administered at t₀ and t₄ₕ in equally divided doses approximately two hours after eating.

Statement 79: The method of any one of Statements 77-78, wherein the mean Cₘₐₓ is from 100% to 150% of the of the mean Cₘₐₓ of a first peak of the equal dose of immediate release liquid solution of sodium oxybate administered at t₀ and t₄ₕ in equally divided doses at least two hours after eating.

Statement 80: The method of any one of Statements 77-79, wherein the mean Cₘₐₓ is from 80% to 100% of the of the mean Cₘₐₓ of a second peak of the equal dose of immediate release liquid solution of sodium oxybate administered at t₀ and t₄ₕ in equally divided doses approximately two hours after eating.

Statement 81: The method of any one of Statements 77-80, wherein the mean Cₘₐₓ is about 50 μg/mL to about 82 μg/mL.

Statement 82: The method of Statement 81, wherein the mean Cₘₐₓ is about 64 μg/mL.

Statement 83: The method of any one of Statements 77-82, wherein the mean AUCᵢₙₜ is about 242 hr*μg/mL when the formulation is administered about 30 minutes after eating.

Statement 84: The method of any one of Statements 77-82, wherein the mean AUCᵢₙₜ is about 273 hr*μg/mL when the formulation is administered about 2 hours after eating.

Statement 85: An oral pharmaceutical composition for the treatment of narcolepsy, cataplexy, or excessive daytime sleepiness comprising gamma-hydroxybutyrate in a unit dose suitable for administration once daily, wherein the composition is dose proportional.

Statement 86: The oral pharmaceutical composition of Statement 85, wherein the composition is suitable for administration in the evening.

Statement 87: The oral pharmaceutical composition of Statement 85, wherein the composition is suitable for administration in the morning.

Statement 88: The oral pharmaceutical composition of any one of Statements 85-87, wherein the composition is dose proportional across 4.5 g, 7.5 g, and 9 g doses of the composition.

Statement 89: The oral pharmaceutical composition of any one of Statements 85-88, wherein the Cₘₐₓ of the composition is proportional across 4.5 g, 7.5 g, and 9 g doses of the composition.

Statement 90: The oral pharmaceutical composition of any one of Statements 85-89, wherein the composition is dose proportional by a factor of 1 to 1.3.

Statement 91: The oral pharmaceutical composition of any one of Statements 88-90, wherein median Tₘₐₓ is between about 1.5 and 2 hours across the increasing doses.

Statement 92: The oral pharmaceutical composition of any one of Statements 88-97, wherein the mean Cₘₐₓ is between about 42.9 and 84.5 μg/mL across the increasing doses.

Statement 93: The oral pharmaceutical composition of any one of Statements 88-92, wherein the mean AUCᵢₙₜ is about 191, 358 and 443 μg·h/mL for the 4.5, 7.5 and 9 g doses respectively.

Statement 94: The oral pharmaceutical composition of any one of Statements 88-93, wherein the mean concentrations at 8 hours are about 4.8, 19.7 and 25.5 μg/mL for the 4.5, 7.5 and 9 g doses respectively.

Statement 95: A method of treating narcolepsy and associated disorders and symptoms in a patient in need thereof comprising: administering an oral pharmaceutical composition comprising gamma-hydroxybutyrate once daily, wherein the composition is dose proportional.

Statement 96: The method of Statement 95, wherein the composition is administered in the evening.

Statement 97: The method of Statement 95, wherein the composition is administered in the morning.

Statement 98: The method of any one of Statements 95-97, wherein the composition is dose proportional across 4.5 g, 7.5 g, and 9 g doses of the composition.

Statement 99: The method of any one of Statements 95-98, wherein the Cmax of the composition is proportional across 4.5 g, 7.5 g, and 9 g doses of the composition.

Statement 100: The method of any one of Statements 95-99, wherein the composition is dose proportional by a factor of 1 to 1.3.

Statement 101: The method of any one of Statements 98-100, wherein median Tₘₐₓ is between about 1.5 and 2 hours across the increasing doses.

Statement 102: The method of any one of Statements 98-101, wherein the mean Cₘₐₓ is between about 42.9 and 84.5 μg/mL across the increasing doses.

Statement 103: The method of any one of Statements 98-102, wherein the mean AUCᵢₙₜ is about 191, 358 and 443 μg·h/mL for the 4.5, 7.5 and 9 g doses respectively.

Statement 104: The method of any one of Statements 98-103, wherein the mean concentrations at 8 hours are about 4.8, 19.7 and 25.5 μg/mL for the 4.5, 7.5 and 9 g doses respectively.

Statement 105: An oral pharmaceutical composition for the treatment of narcolepsy, cataplexy, or excessive daytime sleepiness comprising gamma-hydroxybutyrate in a unit dose suitable for administration once daily, wherein most adverse events (AEs) occur close to Tₘₐₓ, during the Cₘₐₓ period.

Statement 106: The oral pharmaceutical composition of Statement 105, wherein most AEs occur 1.5-2 hours after dosing.

Statement 107: The oral pharmaceutical composition of Statements 105 or 106, wherein administration of the oral pharmaceutical composition less than two hours after eating results in fewer AEs than administration of the oral pharmaceutical composition at least two hours after eating.

Statement 108: The oral pharmaceutical composition of any one of Statements 105-107, wherein the oral pharmaceutical composition has a more favorable safety profile as compared to an equal dose of immediate release liquid solution of sodium oxybate administered at t₀ and t₄ₕ in equally divided doses.

Statement 109: The oral pharmaceutical composition of any one of Statements 105-108, wherein administration of the oral pharmaceutical composition once daily results in fewer AEs than administration of an equal dose of immediate release liquid solution of sodium oxybate administered at t₀ and t₄ₕ in equally divided doses.

Statement 110: A method of treating narcolepsy and associated disorders and symptoms in a patient in need thereof comprising: administering an oral pharmaceutical composition comprising gamma-hydroxybutyrate once daily, wherein most AEs occur close to Tₘₐₓ, during the Cₘₐₓ period.

Statement 111: The method of Statement 110, wherein most AEs occur 1.5-2 hours after dosing.

Statement 112: The method of Statements 110 or 111, wherein administration of the oral pharmaceutical composition less than two hours after eating results in fewer AEs than administration of the oral pharmaceutical composition at least two hours after eating.

Statement 113: The method of any one of Statements 110-112, wherein the oral pharmaceutical composition has a more favorable safety profile as compared to an equal dose of immediate release liquid solution of sodium oxybate administered at t₀ and t₄ₕ in equally divided doses.

Statement 114: The method of any one of Statements 110-113, wherein administration of the oral pharmaceutical composition once daily results in fewer AEs than administration of an equal dose of immediate release liquid solution of sodium oxybate administered at t₀ and t₄ₕ in equally divided doses.

Statement 115: The method of any one of Statements 110-114, wherein the amount of AEs are reduced over an 8 hour, 12 hour, 16 hour, 20 hour, 24 hour, and/or 48 hour time period following administration of the oral pharmaceutical composition, as compared to an equal dose of an immediate release sodium oxybate formulation administered more frequently than once-daily.

Statement 116: A method of reducing the amount of adverse events (AEs) in a patient with narcolepsy, cataplexy, or excessive daytime sleepiness comprising: administering an oral pharmaceutical composition comprising gamma-hydroxybutyrate once daily, wherein the gamma-hydroxybutyrate composition has fewer Cₘₐₓ periods than an equal dose of immediate release sodium oxybate formulation administered more frequently than once-daily.

Statement 117: The method of Statement 116, wherein administration of the gamma-hydroxybutyrate composition results in fewer adverse events and fewer Cₘₐₓ periods over an 8 hour, 12 hour, 16 hour, 20 hour, 24 hour, and/or 48 hour time period, as compared to the immediate release sodium oxybate formulation administered more frequently than once-daily.

Statement 118: The method of Statements 116 or 117, wherein the gamma-hydroxybutyrate composition has a superior safety profile compared to the immediate release sodium oxybate formulation administered more frequently than once-daily.

Statement 119: A method of treating a human patient in need thereof, the method comprising: forming a mixture comprising a pharmaceutical composition comprising gamma-hydroxybutyrate and a soft food vehicle; and orally administering the mixture to the human patient after the forming, wherein the orally administering and the forming occur only once nightly.

Statement 120: The method of statement 119, wherein the pharmaceutical composition comprises an amount of gamma-hydroxybutyrate equivalent to from 3.0 g to 12.0 g of sodium oxybate.

Statement 121: The method of statement 119, wherein the pharmaceutical composition comprises immediate-release and modified-release portions, each of which comprises gamma-hydroxybutyrate.

Statement 122: The method of statement 119, wherein the pharmaceutical composition is a dry particulate formulation or a powdered formulation prior to the forming.

Statement 123: The method of statement 119, wherein the mixture has a pH of less than 6.0.

Statement 124: The method of statement 123, wherein the pH of the suspension is maintained for at least 5 minutes after the forming.

Statement 125: The method of statement 124, wherein the pH of the suspension is maintained for up to 30 minutes after the forming.

Statement 126: The method of statement 123, wherein the pH of the mixture is 5.3 to 5.5.

Statement 127: The method of statement 119, wherein the soft food vehicle is selected from the group consisting of pudding, yogurt, and applesauce.

Statement 128: The method of statement 119, wherein the administering occurs within 30 minutes of the forming.

Statement 129: The method of statement 119, wherein the pharmaceutical composition further comprises hydrogenated vegetable oil, magnesium stearate, methacrylic acid copolymer, and povidone.

Statement 130: The method of statement 129, wherein the pharmaceutical composition further comprises a suspending or viscosifying agent selected from the group consisting of xanthan gum, carrageenan gum, gellan gum, guar gum, sodium alginate, calcium alginate, agar, sodium carboxymethyl cellulose, microcrystalline cellulose, hydroxyethyl cellulose, hydroxypropylmethyl cellulose, and mixtures thereof.

Statement 131: The method of statement 130, wherein the pharmaceutical composition further comprises an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid, or a combination thereof.

Statement 132: The method of statement 119, wherein the soft food vehicle does not have a material impact on a dissolution profile of the pharmaceutical composition.

Statement 133: The method of statement 119, wherein the mixture is bioequivalent to when the pharmaceutical composition is suspended in water.

Statement 134: The method of statement 119, wherein the pharmaceutical composition releases an amount of gamma-hydroxybutyrate into the bloodstream equivalent to from 3.0 g to 12.0 g of sodium oxybate.

Statement 135: A method of treating a disorder treatable with gamma-hydroxybutyrate in a human in need thereof, the method comprising: opening a sachet containing a single daily dose of a pharmaceutical composition, wherein the pharmaceutical composition comprises an amount of gamma-hydroxybutyrate equivalent to from 3.0 to 12.0 g of sodium oxybate; mixing the pharmaceutical composition with a soft food vehicle; and orally administering the mixture, wherein the opening, mixing, and orally administering occur only once daily.

Statement 136: The method of statement 135, wherein the pharmaceutical composition comprises immediate-release and modified-release portions.

Statement 137: The method of statement 135, wherein the soft food vehicle is selected from the group consisting of pudding, yogurt, and applesauce.

Statement 138: The method of statement 135, wherein the soft food vehicle does not have a material impact on a dissolution profile of the pharmaceutical composition.

Statement 139: The method of statement 135, wherein the mixture is bioequivalent to when the pharmaceutical composition is suspended in water.

Statement 140: The method of statement 135, wherein the pH of the mixture is 5.0 to 6.0.

Statement 141: The method of statement 135, wherein the pH of the mixture is 5.2 to 5.7.

Statement 142: The method of statement 135, wherein the pH of the mixture is 5.3 to 5.5.

Statement 143: A pharmaceutical mixture comprising: an immediate release portion comprising gamma-hydroxybutyrate; a modified release portion comprising gamma-hydroxybutyrate; and a soft food vehicle, wherein the ratio of gamma-hydroxybutyrate in the immediate release portion and the modified release portion is from 10/90 to 65/35.

Statement 144: The pharmaceutical mixture of statement 143, further comprising a suspending or viscosifying agent selected from the group consisting of xanthan gum, carrageenan gum, gellan gum, guar gum, sodium alginate, calcium alginate, agar, sodium carboxymethyl cellulose, microcrystalline cellulose, hydroxyethyl cellulose, hydroxypropylmethyl cellulose, and mixtures thereof.

Statement 145: The pharmaceutical mixture of statement 143, further comprising an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid

Statement 146: The pharmaceutical mixture of statement 143, wherein the soft food vehicle is selected from the group consisting of pudding, yogurt, and applesauce.

Statement 147: The pharmaceutical mixture of statement 143, wherein the pH of the mixture is 5.0 to 6.0.

Statement 148: The pharmaceutical mixture of statement 143, wherein the pH of the mixture is 5.2 to 5.7.

Statement 149: The pharmaceutical mixture of statement 143, wherein the pH of the mixture is 5.3 to 5.5.

Statement 150: A pharmaceutical mixture comprising a soft food vehicle, an immediate release portion, and a modified release portion, wherein the immediate release portion comprises particles comprising gamma-hydroxybutyrate, wherein the modified release portion comprises particles comprising gamma-hydroxybutyrate and a coating, and wherein the coating comprises: a polymer carrying free carboxylic groups, and a hydrophobic compound having a melting point equal or greater than 40° C.

Statement 151: A method of treating a human patient in need thereof, the method comprising: orally administering a pharmaceutical mixture to a human patient, the pharmaceutical mixture comprising a soft food vehicle and a composition comprising amount of gamma-hydroxybutyrate equivalent to from 3.0 g to 12.0 g of sodium oxybate, and wherein the composition comprises immediate-release and modified-release portions, each of which comprises gamma-hydroxybutyrate, and wherein the composition comprises a dry particulate or a powder; and wherein the orally administering occurs only once nightly, and wherein the composition has a pH of ≤6.0.

Statement 152: The method of statement 151, wherein the pH of the composition is 5.0 to 5.9.

Statement 153: The method of statement 151, wherein the pH of the composition is 5.2 to 5.7.

Statement 154: The method of statement 151, wherein the pH of the composition is 5.3 to 5.5.

Statement 155: The method of statement 151, wherein the soft food vehicle does not have a material impact on a dissolution profile of the composition.

Statement 156: The method of statement 151, wherein the mixture is bioequivalent to when the composition is suspended in water.

Statement 157: A method of treating a human patient in need thereof, the method comprising: orally administering a pH-adjusted composition to a human patient, the composition comprising a soft food vehicle and an amount of gamma-hydroxybutyrate equivalent to from 3.0 g to 12.0 g of sodium oxybate, and wherein the composition comprises immediate-release and modified-release portions, each of which comprises gamma-hydroxybutyrate, and wherein the amount of gamma-hydroxybutyrate equivalent to from 3.0 g to 12.0 g of sodium oxybate comprises a dry particulate or a powder; wherein the orally administering occurs only once nightly, and wherein the pH of the composition is between 5.0 to 6.0 at administration.

Statement 158: The method of statement 157, wherein the pH of the composition is 5.1 to 5.9 at administration.

Statement 159: The method of statement 157, wherein the pH of the composition is 5.2 to 5.7 at administration.

Statement 160: The method of statement 157, wherein the pH of the composition is 5.3 to 5.5 at administration.

Statement 161: The method of statement 157, wherein the treatment is for narcolepsy, cataplexy, excessive daytime sleepiness, or a sleep related disorder.

Statement 162: The method of statement 157, wherein the soft food vehicle does not have a material impact on a dissolution profile of the composition.

Statement 163: The method of statement 157, wherein the mixture is bioequivalent to when the amount of gamma-hydroxybutyrate equivalent to from 3.0 g to 12.0 g of sodium oxybate is suspended in water.

What is claimed is:

1. A method of treating a human patient in need thereof, the method comprising:
forming a mixture comprising a pharmaceutical composition comprising gamma-hydroxybutyrate and a soft food vehicle; and
orally administering the mixture to the human patient after the forming, wherein the orally administering and the forming occur only once nightly.

2. The method of claim 1, wherein the pharmaceutical composition comprises an amount of gamma-hydroxybutyrate equivalent to from 3.0 g to 12.0 g of sodium oxybate.

3. The method of claim 1, wherein the pharmaceutical composition comprises immediate-release and modified-release portions, each of which comprises gamma-hydroxybutyrate.

4. The method of claim 1, wherein the pharmaceutical composition is a dry particulate formulation or a powdered formulation prior to the forming.

5. The method of claim 1, wherein the mixture has a pH of less than 6.0.

6. The method of claim 5, wherein the pH of the suspension is maintained for at least 5 minutes after the forming.

7. The method of claim 6, wherein the pH of the suspension is maintained for up to 30 minutes after the forming.

8. The method of claim 5, wherein the pH of the mixture is 5.3 to 5.5.

9. The method of claim 1, wherein the soft food vehicle is selected from the group consisting of pudding, yogurt, and applesauce.

10. The method of claim 1, wherein the administering occurs within 30 minutes of the forming.

11. The method of claim 1, wherein the pharmaceutical composition further comprises hydrogenated vegetable oil, magnesium stearate, methacrylic acid copolymer, and povidone.

12. The method of claim 7, wherein the pharmaceutical composition further comprises a suspending or viscosifying agent selected from the group consisting of xanthan gum, carrageenan gum, gellan gum, guar gum, sodium alginate, calcium alginate, agar, sodium carboxymethyl cellulose, microcrystalline cellulose, hydroxyethyl cellulose, hydroxypropylmethyl cellulose, and mixtures thereof.

13. The method of claim 8, wherein the pharmaceutical composition further comprises an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid, or a combination thereof.

14. The method of claim 1, wherein the soft food vehicle does not have a material impact on a dissolution profile of the pharmaceutical composition.

15. The method of claim 1, wherein the mixture is bioequivalent to when the pharmaceutical composition is suspended in water.

16. The method of claim 1, wherein the pharmaceutical composition releases an amount of gamma-hydroxybutyrate into the bloodstream equivalent to from 3.0 g to 12.0 g of sodium oxybate.

17. A method of treating a disorder treatable with gamma-hydroxybutyrate in a human in need thereof, the method comprising:
opening a sachet containing a single daily dose of a pharmaceutical composition, wherein the pharmaceutical composition comprises an amount of gamma-hydroxybutyrate equivalent to from 3.0 to 12.0 g of sodium oxybate;
mixing the pharmaceutical composition with a soft food vehicle; and
orally administering the mixture, wherein the opening, mixing, and orally administering occur only once daily.

18. The method of claim 17, wherein the pharmaceutical composition comprises immediate-release and modified-release portions.

19. The method of claim 17, wherein the soft food vehicle is selected from the group consisting of pudding, yogurt, and applesauce.

20. The method of claim 17, wherein the soft food vehicle does not have a material impact on a dissolution profile of the pharmaceutical composition.

21. The method of claim 17, wherein the mixture is bioequivalent to when the pharmaceutical composition is suspended in water.

22. The method of claim 17, wherein the pH of the mixture is 5.0 to 6.0.

23. The method of claim 17, wherein the pH of the mixture is 5.2 to 5.7.

24. The method of claim 17, wherein the pH of the mixture is 5.3 to 5.5.

25. A pharmaceutical mixture comprising:
an immediate release portion comprising gamma-hydroxybutyrate;
a modified release portion comprising gamma-hydroxybutyrate; and
a soft food vehicle,
wherein the ratio of gamma-hydroxybutyrate in the immediate release portion and the modified release portion is from 10/90 to 65/35.

26. The pharmaceutical mixture of claim 25, further comprising a suspending or viscosifying agent selected from the group consisting of xanthan gum, carrageenan gum, gellan gum, guar gum, sodium alginate, calcium alginate, agar, sodium carboxymethyl cellulose, microcrystalline cellulose, hydroxyethyl cellulose, hydroxypropylmethyl cellulose, and mixtures thereof.

27. The pharmaceutical mixture of claim 25, further comprising an acidifying agent selected from the group consisting of malic acid, citric acid, tartaric acid, adipic acid, boric acid, maleic acid, phosphoric acid, ascorbic acid, oleic acid, capric acid, caprylic acid, and benzoic acid.

28. The pharmaceutical mixture of claim 25, wherein the soft food vehicle is selected from the group consisting of pudding, yogurt, and applesauce.

29. The pharmaceutical mixture of claim 25, wherein the pH of the mixture is 5.0 to 6.0.

30. The pharmaceutical mixture of claim 25, wherein the pH of the mixture is 5.2 to 5.7.

31. The pharmaceutical mixture of claim 25, wherein the pH of the mixture is 5.3 to 5.5.

32. A pharmaceutical mixture comprising a soft food vehicle, an immediate release portion, and a modified release portion, wherein the immediate release portion comprises particles comprising gamma-hydroxybutyrate, wherein the modified release portion comprises particles comprising gamma-hydroxybutyrate and a coating, and wherein the coating comprises:
a polymer carrying free carboxylic groups, and
a hydrophobic compound having a melting point equal or greater than 40° C.

33. A method of treating a human patient in need thereof, the method comprising:
orally administering a pharmaceutical mixture to a human patient, the pharmaceutical mixture comprising a soft food vehicle and a composition comprising amount of gamma-hydroxybutyrate equivalent to from 3.0 g to 12.0 g of sodium oxybate, and wherein the composition comprises immediate-release and modified-release portions, each of which comprises gamma-hydroxybutyrate, and wherein the composition comprises a dry particulate or a powder; and
wherein the orally administering occurs only once nightly, and wherein the composition has a pH of ≤6.0.

34. The method of claim 33, wherein the pH of the composition is 5.0 to 5.9.

35. The method of claim 33, wherein the pH of the composition is 5.2 to 5.7.

36. The method of claim 33, wherein the pH of the composition is 5.3 to 5.5.

37. The method of claim 33, wherein the soft food vehicle does not have a material impact on a dissolution profile of the composition.

38. The method of claim 33, wherein the mixture is bioequivalent to when the composition is suspended in water.

39. A method of treating a human patient in need thereof, the method comprising:
orally administering a pH-adjusted composition to a human patient, the composition comprising a soft food vehicle and an amount of gamma-hydroxybutyrate equivalent to from 3.0 g to 12.0 g of sodium oxybate, and wherein the composition comprises immediate-release and modified-release portions, each of which comprises gamma-hydroxybutyrate, and wherein the amount of gamma-hydroxybutyrate equivalent to from 3.0 g to 12.0 g of sodium oxybate comprises a dry particulate or a powder;
wherein the orally administering occurs only once nightly, and wherein the pH of the composition is between 5.0 to 6.0 at administration.

40. The method of claim 39, wherein the pH of the composition is 5.1 to 5.9 at administration.

41. The method of claim 39, wherein the pH of the composition is 5.2 to 5.7 at administration.

42. The method of claim 39, wherein the pH of the composition is 5.3 to 5.5 at administration.

43. The method of claim 39, wherein the treatment is for narcolepsy, cataplexy, excessive daytime sleepiness, or a sleep related disorder.

44. The method of claim 39, wherein the soft food vehicle does not have a material impact on a dissolution profile of the composition.

45. The method of claim 39, wherein the mixture is bioequivalent to when the amount of gamma-hydroxybutyrate equivalent to from 3.0 g to 12.0 g of sodium oxybate is suspended in water.
